(19) United States
(12) Patent Application Publication
(10) Pub. No.: US 2025/0320289 A1
(43) Pub. Date: Oct. 16, 2025

(54) Identification of B-Cell and T-Cell Receptor Chains for Diagnosis and Treatment of Inflammatory Diseases

(71) Applicant: Easemedcontrol GmbH & Co KG, Munich (DE)

(72) Inventors: Andreas Habenicht, Munich (DE); Changjun Yin, Munich (DE); Chuankai Zhang, Guangdong (CH)

(73) Assignee: Easemedcontrol GmbH & Co KG, Munich (DE)

(21) Appl. No.: 18/574,033

(22) PCT Filed: Mar. 17, 2022
(86) PCT No.: PCT/EP2022/056978

§ 371 Date: Dec. 24, 2023

(30) Foreign Application Priority Data
Apr. 26, 2021 (EP) 21170464.8

(51) Int. Cl. C07K 16/28; A61K 49/00; C07K 14/725

(52) U.S. Cl. CPC C07K 16/28; A61K 49/0058; C07K 14/7051; C07K 2317/565

(57) ABSTRACT

BCR chains or fragments thereof and TCR chains or fragments thereof and their use for the diagnosis and therapy of unresolved inflammatory diseases, in particular atherosclerosis.

### CROSS-REFERENCES TO RELATED APPLICATIONS

This Non-Provisional Patent Application claims the benefit of and priority to PCT/EP2022/056978, filed Mar. 17, 2022, entitled “Identification of B-Cell and T-Cell Receptor Chains for Diagnosis and Treatment of Inflammatory Diseases,” which claims the benefit of and priority to EP Application Serial No. 21170464.8, filed Apr. 26, 2021, entitled “Identification of B-Cell and T-Cell Receptor Chains for Diagnosis and Treatment of Inflammatory Diseases,” the entire contents of both applications of which are hereby incorporated herein by reference.

### REFERENCE TO AN ELECTRONIC SEQUENCE LISTING

The contents of the electronic sequence listing (5010-1-US-SEQ.txt; Size: 426,533 bytes; and Date of Creation: Jun. 12, 2025, is herein incorporated by reference in its entirety.

### BACKGROUND

The present invention relates to B cell receptor (BCR) chains and T cell receptor (TCR) chains which can be used in the diagnosis and therapy of inflammatory diseases, in particular atherosclerosis.

The human immune system utilizes different defense lines to achieve protection against a multiplicity of diseases

B cells, also known as B lymphocytes, are a type of white blood cell of the lymphocyte subtype. They function in the humoral immunity component of the adaptive immune system by secreting antibodies while other B cells are components of the innate immune system also contributing in defense tasks. Additionally, B cells present antigens (they and others are also classified as professional antigen-presenting cells (APCs)) and secrete cytokines.

The BCR is a transmembrane protein on the surface of a B cell. BCRs are composed of immunoglobulin molecules that form a type 1 transmembrane receptor protein, and are typically located on the outer surface of these lymphocyte cells. Through biochemical signaling and by physically acquiring antigens from the immune synapses, the BCR controls the activation of the B cell. B cells are able to gather and grab antigens by engaging biochemical modules for receptor clustering, cell spreading, generation of pulling forces, and receptor transport, which eventually culminates in endocytosis and antigen presentation.

The BCRs binding moiety is composed of a membrane-bound antibody that, like all antibodies, has a uniquely determined antigen-binding site. The BCR of each B cell is unique for this B cell. The BCR for an antigen is a significant sensor that is required for B cell activation, survival, and development. A B cell is activated by its first encounter with an antigen that binds to its receptor (its “cognate antigen”), the cell proliferates and differentiates to generate a population of antibody-secreting plasma B cells or a population of memory B cells that are ready to produce large amounts of antibodies upon repeated antigen interaction such as that typically seen in vaccination upon reinfection with a pathogen such as a virus. These circumstances can protect the host for long periods of time.

Identification of distinct BCR sequences and their subsequent function in the immune system has been difficult in the past but new technologies have greatly facilitated their discovery which allowed the present invention coupled with a new strategy to identify BCRs and TCRs from advanced atherosclerotic mice and translate these data to human patients.

The TCR is a protein complex found on the surface of T cells, or T lymphocytes, that is responsible for recognizing fragments of antigen as peptides bound to major histocompatibility complex (MHC) molecules. The binding between TCR and antigen peptides is of relatively low affinity and is degenerate: that is, many TCRs recognize the same antigen peptide and many antigen peptides are recognized by the same TCR.

The TCR is composed of two different protein chains (that is, it is a hetero dimer). In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD).

A unique feature of T cells is their ability to discriminate between peptides derived from healthy, endogenous cells and peptides from foreign or abnormal (e.g. infected or cancerous) cells in the body.

The lymphoid system is an organ system in vertebrates that is part of the hematopoietic system. One main function is that of immune defense. Lymph is very similar to blood plasma, in that it contains waste products and cellular debris, together with bacteria and proteins. The cells of the lymph are mostly lymphocytes. Associated lymphoid organs are composed of lymphoid tissue, and are the sites either of lymphocyte production or of lymphocyte activation. There is a primary lymphoid system (bone marrow and thymus) and a secondary lymphoid system (lymph nodes, spleen, peyer's patches and more). These lymphoid systems emerge during development.

Tertiary lymphoid organs (TLOs) emerge in tissues in response to non-resolving inflammation in adult organisms. Some of these diseases are associated with disease-associated and disease-affecting autoimmunity. Therefore, TLOs are largely unique for diseases and are not found in healthy individuals.

Artery tertiary lymphoid organs (ATLOs) are found in the aorta adventitia of diseased cardiovascular tissues. ATLOs are organized into T cell areas and B cell follicles containing follicular dendritic cells (FDCs) in activated germinal centers where autoimmune B2 or T cells may be found.

Atherosclerotic arteries harbour distinct immune cell infiltrates in plaques in both the intima and the adventitia. During progression of atherosclerosis the immune system reacts by forming ATLOs in response to the plaque buildup. This may be taken as support for the hypothesis that autoimmune responses may be organized in ATLOs.

The majority of chronic diseases show one or more components of autoreactivity of the immune system such as autoreactive antibodies and/or autoreactive T cells.

As of today, it is not yet known whether or not atherosclerosis (which is used herein as a representative for inflammatory diseases) is a bona fide autoimmune disease involving autoimmune B2 cells and/or autoimmune T-cells that control disease progression. Neither is it known where atherosclerosis affecting autoimmune lymphocytes are generated.

Atherosclerosis causes plaque formation in the arteries and is one of the major causes for thrombisis, ischemic heart diseases and stroke. Nevertheless, the knowledge of additional antigenic triggers and their impact in disease progression remains very limited.

Therefore, despite of progress made in understandingatherosclerosis new and more efficient targeting agents for the diagnosis and treatment of atherosclerosis are needed. There is an unmet medical need fore identifying novel biomarkers that can be efficiently used to detect atherosclerotic plaques thus constituting the basis for developing new clinical tools dedicated to treatment and therapy of human atherosclerosis.

### SUMMARY

It was therefore an object of the present invention to provide novel diagnostic and therapeutic tools and products for the diagnosis and treatment of atherosclerosis and other inflammatory diseases. Therefore, an object of the invention was to identify monoclonal antibodies (encoded in the BCRs) and T cells which specifically react with arterial wall-derived tissues including immune cells thereby affecting disease outcome encoded in the TCRs.

The cloning of atherosclerosis-specific BCRs or the identification of disease-specific T cells allows generation of atherosclerosis-specific B or T lymphocytes, the identification of atherosclerosis-specific autoantigens and the subsequent generation of vaccines and antibody treatments as well as dignostics to diagnose disease stages in the living patient before complication including heart attacks and strokes or death develop.

This object has been achieved by the BCR receptor chains or fragments thereof in accordance with claim 1 and T-cell receptor (TCR) chains or fragment tehreof in accordance with claim 6. Preferred embodiments of the invention are set forth in the dependent claims and the detailed specification hereinafter.

The present invention in a first embodiment provides B-cell receptor (BCR) chains or fragments thereof comprising heavy chain variable regions (IgH) and light chain variable regions (IgL) wherein said heavy chain variable regions (IgH) comprise complementary determining regions IgH CDR1, IgH CDR2 and IgH CDR3 and wherein said light chain variable regions comprise complimentary determining regions IgL CDR1, IgL CDR2 and IgL CDR3 wherein IgH CDR1 comprises amino acid sequences selected from SEQ-ID Nos. 1 to 58, LgH CDR2 comprises sequences selected from SEQ-ID Nos. 59 to 117, LgH CDR3 comprises sequences selected from SEQ-ID Nos. 117 to 174, IGL CDR1 comprises sequences selected from SEQ-ID Nos. 233 to 290 and IgL CDR3 comprises sequences selected from SEQ-IDs No. 291 to 348 in accordance with Table 1:

TABLE 1

| | SEQ-ID | SEQ-ID | SEQ-ID | SEQ-ID | SEQ-ID |
|---|---|---|---|---|---|
| | IgH | IgH | IgH | IgL | IgL |
| BCR No. | CDR1 | CDR2 | CDR3 | CDR1 | CDR3 |
| BCR1 | 1 | 59 | 117 | 233 | 291 |
| BCR2 | 2 | 60 | 118 | 234 | 292 |
| BCR3 | 3 | 61 | 119 | 235 | 293 |
| BCR4 | 4 | 62 | 120 | 236 | 294 |
| BCR5 | 5 | 63 | 121 | 237 | 295 |
| BCR6 | 6 | 64 | 122 | 238 | 296 |
| BCR7 | 7 | 65 | 123 | 239 | 297 |
| BCR8 | 8 | 66 | 124 | 240 | 298 |
| BCR9 | 9 | 67 | 125 | 241 | 299 |
| BCR10 | 10 | 68 | 126 | 242 | 300 |
| BCR11 | 11 | 69 | 127 | 243 | 301 |
| BCR12 | 12 | 70 | 128 | 244 | 302 |
| BCR13 | 13 | 71 | 129 | 245 | 303 |
| BCR14 | 14 | 72 | 130 | 246 | 304 |
| BCR15 | 15 | 73 | 131 | 247 | 305 |
| BCR16 | 16 | 74 | 132 | 248 | 306 |
| BCR17 | 17 | 75 | 133 | 249 | 307 |
| BCR18 | 18 | 76 | 134 | 250 | 308 |
| BCR19 | 19 | 77 | 135 | 251 | 309 |
| BCR20 | 20 | 78 | 136 | 252 | 310 |
| BCR21 | 21 | 79 | 137 | 253 | 311 |
| BCR22 | 22 | 80 | 138 | 254 | 312 |
| BCR23 | 23 | 81 | 139 | 255 | 313 |
| BCR24 | 24 | 82 | 140 | 256 | 314 |
| BCR25 | 25 | 83 | 141 | 257 | 315 |
| BCR26 | 26 | 84 | 142 | 258 | 316 |
| BCR27 | 27 | 85 | 143 | 259 | 317 |
| BCR28 | 28 | 86 | 144 | 260 | 318 |
| BCR29 | 29 | 87 | 145 | 261 | 319 |
| BCR30 | 30 | 88 | 146 | 262 | 320 |
| BCR31 | 31 | 89 | 147 | 263 | 321 |
| BCR32 | 32 | 90 | 148 | 264 | 322 |
| BCR33 | 33 | 91 | 149 | 265 | 323 |
| BCR34 | 34 | 92 | 150 | 266 | 324 |
| BCR35 | 35 | 93 | 151 | 267 | 325 |
| BCR36 | 36 | 94 | 152 | 268 | 326 |
| BCR37 | 37 | 95 | 153 | 269 | 327 |
| BCR38 | 38 | 96 | 154 | 270 | 328 |
| BCR39 | 39 | 97 | 155 | 271 | 329 |
| BCR40 | 40 | 98 | 156 | 272 | 330 |
| BCR41 | 41 | 99 | 157 | 273 | 331 |
| BCR42 | 42 | 100 | 158 | 274 | 332 |
| BCR43 | 43 | 101 | 159 | 275 | 333 |
| BCR44 | 44 | 102 | 160 | 276 | 334 |
| BCR45 | 45 | 103 | 161 | 277 | 335 |
| BCR46 | 46 | 104 | 162 | 278 | 336 |
| BCR47 | 47 | 105 | 163 | 279 | 337 |
| BCR48 | 48 | 106 | 164 | 280 | 338 |
| BCR49 | 49 | 107 | 165 | 281 | 339 |
| BCR50 | 50 | 108 | 166 | 282 | 340 |
| BCR51 | 51 | 109 | 167 | 283 | 341 |
| BCR52 | 52 | 110 | 168 | 284 | 342 |
| BCR53 | 53 | 111 | 169 | 285 | 343 |
| BCR54 | 54 | 112 | 170 | 286 | 344 |
| BCR55 | 55 | 113 | 171 | 287 | 345 |
| BCR56 | 56 | 114 | 172 | 288 | 346 |
| BCR57 | 57 | 115 | 173 | 289 | 347 |
| BCR58 | 58 | 116 | 174 | 290 | 348 |

In a further embodiment the present invention provides BCR chains or fragments thereof in accordance with claim 1 (as listed in Table 1) additionally comprising IgH Full Length sequences selected from SEQ-ID Nos. 175 to 232 or amino acid sequences derived therefrom with a degree of sequence identity in the range of from 90 to 99.7% to SEQ-ID Nos. 175 to 232 in accordance with Table 2 (designated as BCR1′ to BCR 58′)

TABLE 2

| BCR No. | SEQ-ID IgH Full length |
|---|---|
| BCR1′ | 175 |
| BCR2′ | 176 |
| BCR3′ | 177 |
| BCR4′ | 178 |
| BCR5′ | 179 |
| BCR6′ | 180 |
| BCR7′ | 181 |
| BCR8′ | 182 |
| BCR9′ | 183 |
| BCR10′ | 184 |
| BCR11′ | 185 |
| BCR12′ | 186 |
| BCR13′ | 187 |
| BCR14′ | 188 |
| BCR15′ | 189 |
| BCR16′ | 190 |
| BCR17′ | 191 |
| BCR18′ | 192 |
| BCR19′ | 193 |
| BCR20′ | 194 |
| BCR21′ | 195 |
| BCR22′ | 196 |
| BCR23′ | 197 |
| BCR24′ | 198 |
| BCR25′ | 199 |
| BCR26′ | 200 |
| BCR27′ | 201 |
| BCR28′ | 202 |
| BCR29′ | 203 |
| BCR30′ | 204 |
| BCR31′ | 205 |
| BCR32′ | 206 |
| BCR33′ | 207 |
| BCR34′ | 208 |
| BCR35′ | 209 |
| BCR36′ | 210 |
| BCR37′ | 211 |
| BCR38′ | 212 |
| BCR39′ | 213 |
| BCR40′ | 214 |
| BCR41′ | 215 |
| BCR42′ | 216 |
| BCR43′ | 217 |
| BCR44′ | 218 |
| BCR45′ | 219 |
| BCR46′ | 220 |
| BCR47′ | 221 |
| BCR48′ | 222 |
| BCR49′ | 223 |
| BCR50′ | 224 |
| BCR51′ | 225 |
| BCR52′ | 226 |
| BCR53′ | 227 |
| BCR54′ | 228 |
| BCR55′ | 229 |
| BCR56′ | 230 |
| BCR57′ | 231 |
| BCR58′ | 232 |

In accordance with a further embodiment, the BCR chains or fragments thereof in accordance with the present invention additionally comprise IgL Full Length sequences selected from SEQ-ID Nos. 349 to 407 or amino acid sequences derived therefrom with a degree of sequence identity in the range of from 90 to 99.7% to SEQ-ID Nos. 349 to 407 in accordance with Table 3 (designated as BCR1″ to BCR58″):

TABLE 3

| BCR No. | SEQ-ID IgL Full Length |
|---|---|
| BCR1″ | 349 |
| BCR2″ | 350 |
| BCR3″ | 351 |
| BCR4″ | 352 |
| BCR5″ | 353 |
| BCR6″ | 354 |
| BCR7″ | 355 |
| BCR8″ | 356 |
| BCR9″ | 357 |
| BCR10″ | 358 |
| BCR11″ | 359 |
| BCR12″ | 360 |
| BCR13″ | 361 |
| BCR14″ | 362 |
| BCR15″ | 363 |
| BCR16″ | 364 |
| BCR17″ | 365 |
| BCR18″ | 366 |
| BCR19″ | 367 |
| BCR20″ | 368 |
| BCR21″ | 369 |
| BCR22″ | 370 |
| BCR23″ | 371 |
| BCR24″ | 372 |
| BCR25″ | 373 |
| BCR26″ | 374 |
| BCR27″ | 375 |
| BCR28″ | 376 |
| BCR29″ | 377 |
| BCR30″ | 378 |
| BCR31″ | 379 |
| BCR32″ | 380 |
| BCR33″ | 381 |
| BCR34″ | 382 |
| BCR35″ | 383 |
| BCR36″ | 384 |
| BCR37″ | 385 |
| BCR38″ | 386 |
| BCR39″ | 387 |
| BCR40″ | 388 |
| BCR41″ | 389 |
| BCR42″ | 390 |
| BCR43″ | 391 |
| BCR44″ | 392 |
| BCR45″ | 393 |
| BCR46″ | 394 |
| BCR47″ | 395 |
| BCR48″ | 396 |
| BCR49″ | 397 |
| BCR50″ | 398 |
| BCR51″ | 399 |
| BCR52″ | 400 |
| BCR53″ | 401 |
| BCR54″ | 402 |
| BCR55″ | 403 |
| BCR56″ | 404 |
| BCR57″ | 405 |
| BCR58″ | 406 |

In accordance with a still further embodiment of the present invention, the BCR chains or fragments thereof in addition to the CDR sequences in accordance with Table 1 comprise an IgL full length amino acid sequence selected from SEQ-ID Nos. 175 to 232 or an amino acid full length sequence derived therefrom with a degree of sequence identity in the range of from 90 to 99.7% to SEQ-ID Nos. 175 to 232 in accordance with Table 2 and an IgL Full Length amino acid sequence selected from SEQ-ID Nos. 349 to 406 or full length amino acid sequences derived from SEQ-ID Nos. 349 to 406 with a degree of sequence identity in the range of from 90 to 99.7% in accordance with Table 3.

In accordance with another embodiment of the present invention the BCR chains or fragments thereof comprising CDR sequences as defined in Table 1 and, optionally, IgL and/or IgH full length sequences in accordance with Table 2 and Table 3, respectively, additionally comprise sequences of three amino acids in complementary determining region IgL CDR2 in accordance with Table 4 (designated as BCR1″′ to BCR 58″′).

TABLE 4

| BCR No. | Amino acid sequence IgL CDR2 |
|---|---|
| BCR1″′ | YAS |
| BCR2″′ | EGN |
| BCR3″′ | RAS |
| BCR4″′ | LAS |
| BCR5″′ | RAS |
| BCR6″′ | AAS |
| BCR7″′ | AAS |
| BCR8″′ | AAS |
| BCR9″′ | YAS |
| BCR10″′ | AAS |
| BCR11″′ | AAS |
| BCR12″′ | AAS |
| BCR13″′ | RAS |
| BCR14″′ | AAS |
| BCR15″′ | GTS |
| BCR16″′ | AAS |
| BCR17″′ | AAS |
| BCR18″′ | AAS |
| BCR19″′ | AAS |
| BCR20″′ | YTS |
| BCR21″′ | STS |
| BCR22″′ | RAS |
| BCR23″′ | AAS |
| BCR24″′ | RAS |
| BCR25″′ | GTN |
| BCR26″′ | GAS |
| BCR27″′ | STS |
| BCR28″′ | YAS |
| BCR29″′ | YAS |
| BCR30″′ | AAS |
| BCR31″′ | KAS |
| BCR32″′ | AAS |
| BCR33″′ | STS |
| BCR34″′ | AAS |
| BCR35″′ | AAS |
| BCR36″′ | RAS |
| BCR37″′ | STS |
| BCR38″′ | YTS |
| BCR39″′ | AAS |
| BCR40″′ | AAS |
| BCR41″′ | STS |
| BCR42″′ | AAS |
| BCR43″′ | IAS |
| BCR44″′ | YTS |
| BCR45″′ | KAS |
| BCR46″′ | NTS |
| BCR47″′ | VAS |
| BCR48″′ | YTS |
| BCR49″′ | YTS |
| BCR50″′ | RTS |
| BCR51″′ | RTS |
| BCR52″′ | YAS |
| BCR53″′ | YTF |
| BCR54″′ | YTS |
| BCR55″′ | IAS |
| BCR56″′ | RAN |
| BCR57″′ | YTS |
| BCR58″′ | YTS |

In Tables 1 to 4 designations BCR1 to BCR58, BCR1′ to BCR58′, BCR1″ to BCR58″ and BCR1″′ to BCR58″′ are used. In this regard BCR1 represents a BCR chain or a fragment thereof comprising the CDR sequences given in Table 1 for BCR1, BCR1′ represents BCR1 plus the full length sequence given in Table 2 for BCR1 or a full length sequence having a degree of sequence identity of from 90 to 99.7% to the SEQ-IDs listed in Table 2, BCR1″ represents BCR1 plus the full length sequence given in Table 3 for BCR1″ or a full length sequence having a degree of sequence identity of from 90 to 99.7% to the SEQ-IDs listed in Table 3. Finally BCR1″′ represents BCR1 plus the amino acid sequence given for BCR1″′ in Table 4. BCR2 to BCR58, BCR2′ to BCR58′, BCR2″ to BCR58″ and BCR1″′ to BCR58″′ are to be interpreted accordingly.

The present invention also comprises BCR chains and fragments thereof which cumulatively comprise the CDR sequences in accordance with Table 1, the full length sequences in accordance with Tables 2 or full length sequences having a degree of sequence identity of from 90 to 99.7% to the SEQ-IDs listed in Table 2, the full length sequences in accordance with Table 3 or full length sequences having a degree of sequence identity of from 90 to 99.7% to the SEQ-IDs listed in Table 3 and the amino acid sequences in accordance with Table 4. These could be designated as BCR1″″ to BCR58″″ in the analogous manner than explained above for BCR1 to BCR1″′.

BCR chain or fragments thereof as defined in Tables 1 and 2 are particularly suitable for use in a method for imaging or diagnosis of atherosclerosis, in particular wherein the method comprises the visualization of atherosclerotic lesions by detection of the BCR chain or fragment thereof previously administered to the patient.

In the course of the present invention it has been found that atherosclerosis is a disease associated with the generation of disease-controlling autoimmune B2 cells. This opens new-and indeed for the first time-possibilities for the diagnosis and therapy of atherosclerosis. Autoimmune B cells and T cells have not yet been achieved in the past but this aim was successfully achieved by using the isolation of germinal center B cells from the adventitia and ATLOs of late stage atherosclerosis mice or T cells from atherosclerotic plaques of the same mice.

ATLOs develop in the artery adventitia and the adventitia of other arteries of aged ApoE^{-/-} mice and human patients at sites afflicted with atherosclerosis. ATLOs are organized into T cell areas and B cell follicles containing follicular dendritic cells (FDCs) in activated germinal centers. Atherosclerosis-specific B2 cell and T cell responses are organized in these ATLOs and can thus be used to diagnose atherosclerosis, to monitor the development of the disease and can serve as basis for new therapeutic treatments. This finding of specific autoimmune lymphocytes significantly narrows down the pool of such autoimmune lymphocytes to be evaluated (with molecular biology tools) in the course of identifying and isolating autoimmune cells that either protect or suppress the disease.

It was thus found that clonal expansion of BCRs occurs in antigen-dependent ways in B2 cells in germinal centers of secondary lymphoid organs or ATLOs. Clonal expansion (the strong and explosive increase of the number of lymphocytes—of both B cells and T cells—in the presence of an infection or other exposure to autoimmune antigens) gives the adaptive immune system its extraordinary power and specificity and in some cases can also result in disease, then called autoimmune disease. This specificity is achieved by the immune system by virtue of two mechanisms: Generation of new unique BCR throughout life and by clonal expansion of TCR-carrying T cells.

The apolipoprotein E-deficient (ApoE^{-/-}) mouse is currently the most popular murine model used for atherosclerotic studies and was also used in the course of the present invention. It was found that ATLOs, spleen and lymph nodes (LNs) in ApoE^{-/-} mice showed a profound BCR clonal expansion when compared to those of aged WT (wild type) mice. A quantitative comparison of the clonality of BCR repertoires of different tissues and individual mice was done by clonal expansion of BCRs from each tissue by determination of the clonal expansion index (also commonly referred to as Gini index). In spleens of ApoE^{-/-} mice CD19+ B cells an increase of the Gini index occurred compared to WT mice. Higher clonal expansion of CD19+ B cells occurred in ATLOs and SLOs in ApoE^{-/-} mice compared to WT mice.

During BCR assembly the BCR undergoes somatic hypermutation by changing its sequence to generate a new and uniques BCR sequence exclusive for the disease in which the BCR is generated and further sequence alterations which determines the nature of the autoimmune B cell in terms of additional sequences called affinity-maturation. The BCR sequences reported here are unknown and most of them are reacting towards atherosclerotic plaque consituents demonstrating their autoimmune nature and uniqueness for atherosclerosis.

The results of the studies in the course of the present invention thus showed that there is higher diversification, somatic hypermutation (SHM) and affinity maturation and isotype switching of BCRs of CD19+B cells in SLOs and ATLOs of aged ApoE^{-/-} mice when compared to WT mice. The invention reported here involves sequences unique to disease and are not found in healthy mice of healthy humans.

These results show that atherosclerosis/hyperlipidemia is associated with enhanced antigen-dependent germinal center reactions in spleen, LNs and ATLOs in ApoE^{-/-} mice resulting in unique BCR sequences specific for atherosclerosis. There is also a systemic enhanced GC reaction in secondary lymphoid organs and ATLOs indicating that ATLOs are the site where germinal center reactions involve arterial wall-specific antigen-dependent B2 activation.

Overall these results show that the BCRs in accordance with the present invention can be used to monitor, diagnose and eventually therapeutically treat atherosclerosis by both vaccination strategies or antibody treament regimens.

In the course of the present invention it was found that atherosclerosis-relevant B2 cell adaptive immune response is organized in germinal centers of secondary lymphoid organs and ATLOs in aged ApoE^{-/-} mice. The germinal centers in secondary lymphoid organs organize adaptive immunity in normal immune homeostasis while those in ATLOs are specifically involved in humoral immune responses against arterial wall-derived autoantigens which is a pillar of our strategic approach to isolate autoimmune BCRs in atherosclerosis.

This was confirmed by cloning and expression of monocolonal antibodies (encoded in the BCRs) using the paired IgH/IgL sequences from germinal center B cells of either secondary lymphoid organs or ATLOs. Single cell PCR and Sanger sequencing were applied to obtain the full length V regions of IgH-IgL. IMGT database was used to identify the subfamilies of IgH and IgL chains.

Single ATLO germinal center B cells (CD19⁺lgD-PNA⁺GL7⁺) were sorted and the full length of Ig heavy and light chains were cloned and sequenced to obtain ATLO GC derived antibodies respectively BCR sequences in the IgH and the IgL part of the antibody. BCR chains or fragments thereof as defined in claims 1 to 2 and referred to as BCR1 to BCR58, BCR1′ to BCR58′, BCR1″ to BCR58″ and BCR1″′ to BCR58″′ in Tables 1 to 4 hereinafter have shown to be particularly suitable for monitoring, diagnosis and treatment of atherosclerosis as same show strongest correlation with and reactivity to atherosclerotic plaques which are the hallmark of atherosclerosis in mice and men.

In particular ATLO derived antibodies BCR56, BCR56′, BCR56″ and BCR56″′ recognized a plaque nuclear auto-antigen in a dose dependent manner and show significantly higher signal intensity in plaques and aorta in aged aged ApoE--mice when compared to aged WT aorta. ATLO derived BCR56, BCR56′, BCR56″ and BCR56″′ also recognize early stages of atherosclerotic plaques in 32 weeks old mice indicating that the diagnostic tools and indeed treatments can be used in young individuals rather than only in aged individuals.

Important for the current invention, mouse derived autoantibodies react with human diseased arteries including plaques that cause disease such as myocardial infarcts, strokes and other deadly diseases.

ATLO derived antibodies BCR56, BCR56′, BCR56″ and BCR56″′ were used for searching for atherosclerosis-relevant auto-antigens and it was found that these antibodies recognized histone proteins. When using BCR56, BCR56′, BCR56″ and BCR56″′ antibody as a bait to capture BCR56, BCR56′, BCR56″ and BCR56″′ antigens from the supernatant of diseased aorta lysates of aged ApoE^{-/-} mice provided proof that BCR56, BCR56′, BCR56″ and BCR56″′ recognized a major band with molecular size 10-15 kD.

From combined data of immunofluorescent staining, Western Blot and mass spectrometry it can be concluded that BCR56, BCR56′, BCR56″ and BCR56″′ antigen(s) locate(s) in the nucleus with the molecular weight 10-15 kD and represent histones. The binding of BCR56, BCR56′, BCR56″ and BCR56″′ to histones was evaluated by various studies with a histone mixture from calf thymus as well as with a mixture of bovine histones. Furthermore, it was found that (BCR56, BCR56′, BCR56″ and BCR56″′) antibodies bind to human recombinant histone mixture in a dose dependent manner. The data indicate that histone H2B is the cognate antigen for ATLO GC-derived antibodies (BCR56, BCR56′, BCR56″ and BCR56″′).

The full mouse recombinant antibody (BCR56″′) was used to distinguish human endogenous immunoglobulins in human tissue sections using anti-mouse IgG secondary antibody. The data of these experiments show that BCR56, BCR56′, BCR56″ and BCR56″′ antigens are present in human carotid plaque sections. The same antigen appears to exist in both murine and human atherosclerotic plaques. The antigens may be released within the diseased arterial wall microenvironment which triggers autoimmune B2 responses in germinals centers of ATLOs in aged ApoE^{-/-} mice. A human histone protein mixture (H2A, H2B, H3.1, H3.2, H3.3, and H4.) was used as antigens to screen serum of WT and ApoE^{-/-} mice during aging. The results obtained show that there is a significantly higher AHA titer in serum in mice with atherosclerosis in adult and aged ApoE^{-/-} mice when compared to WT mice. 8 weeks old ApoE^{-/-} mice do not develop atherosclerosis, there is no significant difference in these titers between 8 weeks WT and ApoE^{-/-} mice lending support to the use of these antibodies to be used as diagnostic markers early in the course of the disease. These data show that serum AHA titer represents a diagnosis marker of atherosclerosis in ApoE^{-/-} mice. The data also show that BCR56, BCR56′, BCR56″ and BCR56″′ autoantigens are present in human atherosclerotic plaques.

This data prompted a study to examine the possibility that what was observed in aged ApoE^{-/-} mice may be translatable to patients with cardiovascular diseases. Serum anti-histone antibodies (AHA) titers from age- and sex-matched asymptomatic (without neurological symptoms) patients (n=31) and symptomatic (with neurological symptoms) patients (n=34) with carotid artery atherosclerosis. Symptomatic patients showed significantly higher AHA titers when compared to asymptomatic patients. In this experiment, 4 healthy control sera were donated from volunteers from colleagues. This unexpected result suggested that histone proteins (including BCR56 antigen) may trigger autoimmune responses in human patients with cardiovascular diseases. Anti-histone antibody diagnostics (including BCR56, BCR56′, BCR56″ and BCR56″′) therefore represent serum biomarkers to diagnose atherosclerosis.

Thus, the data overall provide evidence as proof of principle that the sequences provided here on BCRs and fragments thereof are a rich source of autoimmune BCRs and that data observed in aged ApoE^{-/-} mice can be translated to patients with atherosclerosis and can be used as diagnostic tools and possibly early during disease development to predict disease progression and guide treatment.

The embodiments of the present invention described hereinbefore relate to BCR chains or fragments thereof specifically binding to atherosclerotic lesions. The BCR chains or fragments thereof in accordeance with the present invention show reactivity against atherosclerotic plaques.

The present invention also encompasses polynucleotides encoding any of the above cited BCR chains or fragments threof, expression vectors comprising such polynucleotides and recombinant host cells comrpising said polynucleotides or said expression vecotors.

A further embodiment of the present invention relates to a TCR chain or fragments thereof for use in the development of T cell mediated atherosclerosis treatments.

To generate a comprehensive view of cellularity and clonality of T cells in atherosclerosis, a transcriptome and TCR repertoire atlas comprising 3310 T cells collected from atherosclerotic plaques, ATLOs, and aorta-draining renal lymph nodes (rLNs) from 3 aged (78-week old) C57BL/6 wild type (WT) and ApoE^{-/-} mice was constructed. 11 T cell subsets were defined according to T cell lineage marker expression, i.e., CD4 as a marker of helper T cells; CD8a for cytotoxic T cells; Foxp3 for regulatory T (Treg) cells; CCR7 as a chemokine receptor and SELL (CD62L), a cell surface selection marker, both involved in recruiting T cells and homing to lymph nodes. CD44 is a marker of T cell activation, the combination of SELL, CD44 and CCR7 is used to discriminate naive, central memory and effector memory T cells; killer cell lectin-like receptor subfamily B member 1C (Klrb1c or NK1.1) for natural killer T cells; TCR gamma constant region 1 (Torg-c1) and TCR delta constant region (Trdc) for γδ T cells; co-expression of Icos, Cxcr5, Sh2d1a is used for follicular helper T (Tfh) cells. 11 T cells subsets were named as follows: cluster 0, CD4 effector regulatory T cells (eTreg, CD4+Foxp3+CD44+Sell−Ccr7low); cluster 1, CD4 Tem (CD4 effector memory T cells, CD4+Foxp3−CD44+Sell−Ccr7low); cluster 2, CD8 Tem (CD8a+CD44+Sell−Ccr7low); cluster 3, CD8 naïve T cell (CD8a+CD44−Sell+Ccr7high); cluster 4, CD8 Tcm (CD8 central memory T cells, CD8a+CD44+Sell+Ccr7int); cluster 5, CD8 Tcm/NKT (CD8a+CD44+Sell+Ccr7intKlrb1c+); cluster 6, CD4 naïve T cells (CD4+CD44−Sell+Ccr7high); cluster 7, CD4 central Treg cells (cTreg, CD4+Foxp3+Ccr7low); cluster 8, γδ T cells (CD4−CD8a−Tcrgc1+Trdc+); cluster 9, follicular T helper cells (Tfh cells, Icos+Cxcr5+Shad1a+), and cluster 10, double negative T cells (DN T cells, CD3e+CD4−CD8a−).

Pathway enrichment analyses were performed based on their transcriptome to validate biological functions for each cluster, i.e. highly expressed genes of cluster 9 (T follicular helper; Tfh cells) are enriched in B cell activation genes, proliferation, homeostasis and germinal center formation; gene expression of cluster 8 (γδ T cells) are enriched in Th17 cell differentiation. The cell proportion of each T cell subset was compared within different tissue microenvironments. LNs obtained from aged WT and ApoE^{-/-} mice showed similarities in majority of T cell subsets percentage consistent with our previous reports using different methods. Moreover, higher percentages of CD8 Tem and γδ T cells has been observed in ATLOs and atherosclerotic plaques.

Double negative (DN) T cells have been reported to suppress inflammatory responses in several immune-related diseases. A small population of DN T cells has been observed in plaques, while its percentage is 10 times higher when compared to its percentages in LNs and ATLOs. Gene set enrichment analysis (GSEA) indicated that complement activation and cholesterol homeostasis pathways are significantly enriched in DN T cells, while inflammatory response and allograft rejection gene sets were negatively correlated. These data suggest that DN T cells play immunosuppressive roles in atherosclerotic plaques. Of note, plaques have less percentages of effector Treg cells and central Treg cells when compared to LNs and ATLOs, which in turn, may contribute to the imbalance of the pro-versus anti-atherogenetic immune responses observed in atherosclerosis. All these data not only provide insight into the pathogenesis of atherosclerosis but also provide unique TCR sequences with the nature of autoimmune T cells which can be used as treatment and diagnostic regimens to treat patients with atherosclerosis.

Individual T cells carry unique antigen-specific, paired-TCRα/β chains, which allows to detect T cell expansion together with their transcriptome in t-SNE projections. We defined one T cell clonotype as T cells (n≥2) carrying the identical paired-TCRα/β CDR3 region. 2798 TCRα chain or TCRβ chain sequences (84.5% of total T cells) and 2092 paired-TCRα/β (63.2% of total T cells) were obtained from a total of 3310 T cells. These data revealed that this is a high-throughput and high-yield method to obtain the paired-TCRα/β at single cell resolution. Moreover, this method allowed us to combine the transcriptomes with the paired-TCRα/β sequences at the single cell resolution, which enabled us to compare transcript expression between expanded T cells vs non-expanded T cells in different tissue microenvironments. 4.72% expanded TCRs in WT LNs, 8.17% and 8.77% expanded TCRs in ApoE^{-/-} LNs and ATLOs, while there were 20.71% expanded TCRs in atherosclerotic plaques. The Shannon diversity index was used to determine TCR diversity, which has a negative correlation with clonality. Our data revealed that atherosclerosis plaques showed less TCR diversity when compared to LNs and ATLOs consistent with the higher percentage of expanded T cells in atherosclerotic plaques. In the next step it was determined which T cell subsets are expanded. t-SNE maps were used to combine transcripts with TCR sequences of each individual cell. The 11 T cell subsets were grouped into four major clusters according to T cell functions, including cluster I: CD4 memory, effector and regulatory T cells; cluster II: CD8 memory and effector T cells; cluster III: naïve T cells, and cluster IV: γδ-T cells. Expanded T cells were limited to memory and effector CD4 and CD8 T cells (clusters I and II). The observation of a lack of expanded T cells in naïve T cells (cluster III) in all tissue microenvironments confirmed the high quality of the data sets. Higher percentages of expanded CD4 memory/effector/regulatory T cells in plaques (15.0%) when compared to WT LNs (8.1%), ApoE^{-/-} LNs (5.6%), and ATLOs (5.0%) were detected. Moreover, the data indicate that there is a higher percentage of expanded CD8 memory/effector T cells in ApoE^{-/-} mice (ApoE^{-/-} LNs 16.2%, ATLOs 17.9%, plaques 38.1%) when compared to WT mice (WT LNs, 5.9%). Taken together, the data show that the percentages of expanded CD4 memory/effector/regulatory T cells in plaques is 2 times higher when compared to WT LNs and 3 times higher when compared to ApoE^{-/-} LNs and ATLOs. Moreover, the percentages of expanded CD8 memory/effector/regulatory T cells in plaques is 6.5 times higher when compared to WT LNs, and 2 times higher when compared to ApoE^{-/-} LNs and ATLOs.

Thus, the present invention provides a bank of clonally uniquely expanded T cells in atherosclerosis including their paired TCR sequences that have been unknown before. The paired-TCR sequences are are a rich source to identify candidates to develop T cell-mediated atherosclerosis treatments.

T-cell receptor (TCR) chain or part thereof comprising a beta chain part (TRBV) and an alpha chain part (TRAV) wherein said beta chain part (TRBV) comprises complementary determining regions TRBV CDR1, TRBV CDR2 and TRBV CDR3 and wherein said alpha chain part TRAV comprises complimentary determining regions TRAV CDR1, TRAV CDR2 and TRAV CDR3 wherein TRBV CDR1 comprises amino acid sequences selected from SEQ-ID Nos. 407 to 477, TRBV CDR2 comprises sequences selected from SEQ-ID Nos. 478 to 548, TRBV CDR3 comprises sequences selected from SEQ-ID Nos.549 to 619, TRAV CDR1 comprises sequences selected from SEQ-ID Nos. 691 to 761, TRAV CDR2 comprises sequences selected from SEQ-ID Nos. 762 to 832 and TRAV CDR3 comprises sequences selected from SEQ-IDs No. 833 to 903 in accordance with Table 5 represent a further embodiment of the present invention.

TABLE 5

| | SEQ-ID | SEQ-ID | SEQ-ID | SEQ-ID | SEQ-ID | SEQ-ID |
|---|---|---|---|---|---|---|
| | TRBV | TRBV | TRBV | TRAV | TRAV | TRAV |
| TCR No. | CDR1 | CDR2 | CDR3 | CDR1 | CDR2 | CDR3 |
| TCR1 | 407 | 478 | 549 | 691 | 762 | 833 |
| TCR2 | 408 | 479 | 550 | 692 | 763 | 834 |
| TCR3 | 409 | 480 | 551 | 693 | 764 | 835 |
| TCR4 | 410 | 481 | 552 | 694 | 765 | 836 |
| TCR5 | 411 | 482 | 553 | 695 | 766 | 837 |
| TCR6 | 412 | 483 | 554 | 696 | 767 | 838 |
| TCR7 | 413 | 484 | 555 | 697 | 768 | 839 |
| TCR8 | 414 | 485 | 556 | 698 | 769 | 840 |
| TCR9 | 415 | 486 | 557 | 699 | 770 | 841 |
| TCR10 | 416 | 487 | 558 | 700 | 771 | 842 |
| TCR11 | 417 | 488 | 559 | 701 | 772 | 843 |
| TCR12 | 418 | 489 | 560 | 702 | 773 | 844 |
| TCR13 | 419 | 490 | 561 | 703 | 774 | 845 |
| TCR14 | 420 | 491 | 562 | 704 | 775 | 846 |
| TCR15 | 421 | 492 | 563 | 705 | 776 | 847 |
| TCR16 | 422 | 493 | 564 | 706 | 777 | 848 |
| TCR17 | 423 | 494 | 565 | 707 | 778 | 849 |
| TCR18 | 424 | 495 | 566 | 708 | 779 | 850 |
| TCR19 | 425 | 496 | 567 | 709 | 780 | 851 |
| TCR20 | 426 | 497 | 568 | 710 | 781 | 852 |
| TCR21 | 427 | 498 | 569 | 711 | 782 | 853 |
| TCR22 | 428 | 499 | 570 | 712 | 783 | 854 |
| TCR23 | 429 | 500 | 571 | 713 | 784 | 855 |
| TCR24 | 430 | 501 | 572 | 714 | 785 | 856 |
| TCR25 | 431 | 502 | 573 | 715 | 786 | 857 |
| TCR26 | 432 | 503 | 574 | 716 | 787 | 858 |
| TCR27 | 433 | 504 | 575 | 717 | 788 | 859 |
| TCR28 | 434 | 505 | 576 | 718 | 789 | 860 |
| TCR29 | 435 | 506 | 577 | 719 | 790 | 861 |
| TCR30 | 436 | 507 | 578 | 720 | 791 | 862 |
| TCR31 | 437 | 508 | 579 | 721 | 792 | 863 |
| TCR32 | 438 | 509 | 580 | 722 | 793 | 864 |
| TCR33 | 439 | 510 | 581 | 723 | 794 | 865 |
| TCR34 | 440 | 511 | 582 | 724 | 795 | 866 |
| TCR35 | 441 | 512 | 583 | 725 | 796 | 867 |
| TCR36 | 442 | 513 | 584 | 726 | 797 | 868 |
| TCR37 | 443 | 514 | 585 | 727 | 798 | 869 |
| TCR38 | 444 | 515 | 586 | 728 | 799 | 870 |
| TCR39 | 445 | 516 | 587 | 729 | 800 | 871 |
| TCR40 | 446 | 517 | 588 | 730 | 801 | 872 |
| TCR41 | 447 | 518 | 589 | 731 | 802 | 873 |
| TCR42 | 448 | 519 | 590 | 732 | 803 | 874 |
| TCR43 | 449 | 520 | 591 | 733 | 804 | 875 |
| TCR44 | 450 | 521 | 592 | 734 | 805 | 876 |
| TCR45 | 451 | 522 | 593 | 735 | 806 | 877 |
| TCR46 | 452 | 523 | 594 | 736 | 807 | 878 |
| TCR47 | 453 | 524 | 595 | 737 | 808 | 879 |
| TCR48 | 454 | 525 | 596 | 738 | 809 | 880 |
| TCR49 | 455 | 526 | 597 | 739 | 810 | 881 |
| TCR50 | 456 | 527 | 598 | 740 | 811 | 882 |
| TCR51 | 457 | 528 | 599 | 741 | 812 | 883 |
| TCR52 | 458 | 529 | 600 | 742 | 813 | 884 |
| TCR53 | 459 | 530 | 601 | 743 | 814 | 885 |
| TCR54 | 460 | 531 | 602 | 744 | 815 | 886 |
| TCR55 | 461 | 532 | 603 | 745 | 816 | 887 |
| TCR56 | 462 | 533 | 604 | 746 | 817 | 888 |
| TCR57 | 463 | 534 | 605 | 747 | 818 | 889 |
| TCR58 | 464 | 535 | 606 | 748 | 819 | 890 |
| TCR59 | 465 | 536 | 607 | 749 | 820 | 891 |
| TCR60 | 466 | 537 | 608 | 750 | 821 | 892 |
| TCR61 | 467 | 538 | 609 | 751 | 822 | 893 |
| TCR62 | 468 | 539 | 610 | 752 | 823 | 894 |
| TCR63 | 469 | 540 | 611 | 753 | 824 | 895 |
| TCR64 | 470 | 541 | 612 | 754 | 825 | 896 |
| TCR65 | 471 | 542 | 613 | 755 | 826 | 897 |
| TCR66 | 472 | 543 | 614 | 756 | 827 | 898 |
| TCR67 | 473 | 544 | 615 | 757 | 828 | 899 |
| TCR68 | 474 | 545 | 616 | 758 | 829 | 900 |
| TCR69 | 475 | 546 | 617 | 759 | 830 | 901 |
| TCR70 | 476 | 547 | 618 | 760 | 831 | 902 |
| TCR71 | 477 | 548 | 619 | 761 | 832 | 903 |

TCR chain or fragment thereof as defined in Table 5 additionally comprising a full length TRBV amino acid sequence selected from SEQ-ID Nos. 678 to 748 or an amino acid sequence derived therefrom with a degree of sequence identity in the range of from 90 to 99,7% of SEQ-ID No. 678 to 748 in its beta chain part in accordance with Table 6 represent a further embodiment of the present invention.

TABLE 6

| TCR No. | SEQ-ID TRBV |
|---|---|
| TCR1′ | 620 |
| TCR2′ | 621 |
| TCR3′ | 622 |
| TCR4′ | 623 |
| TCR5′ | 624 |
| TCR6′ | 625 |
| TCR7′ | 626 |
| TCR8′ | 627 |
| TCR9′ | 628 |
| TCR10′ | 629 |
| TCR11′ | 630 |
| TCR12′ | 631 |
| TCR13′ | 632 |
| TCR14′ | 633 |
| TCR15′ | 634 |
| TCR16′ | 635 |
| TCR17′ | 636 |
| TCR18′ | 637 |
| TCR19′ | 638 |
| TCR20′ | 639 |
| TCR21′ | 640 |
| TCR22′ | 641 |
| TCR23′ | 642 |
| TCR24′ | 643 |
| TCR25′ | 644 |
| TCR26′ | 645 |
| TCR27′ | 646 |
| TCR28′ | 647 |
| TCR29′ | 648 |
| TCR30′ | 649 |
| TCR31′ | 650 |
| TCR32′ | 651 |
| TCR33′ | 652 |
| TCR34′ | 653 |
| TCR35′ | 654 |
| TCR36′ | 655 |
| TCR37′ | 656 |
| TCR38′ | 657 |
| TCR39′ | 658 |
| TCR40′ | 659 |
| TCR41′ | 660 |
| TCR42′ | 661 |
| TCR43′ | 662 |
| TCR44′ | 663 |
| TCR45′ | 664 |
| TCR46′ | 665 |
| TCR47′ | 666 |
| TCR48′ | 667 |
| TCR49′ | 668 |
| TCR50′ | 669 |
| TCR51′ | 670 |
| TCR52′ | 671 |
| TCR53′ | 672 |
| TCR54′ | 673 |
| TCR55′ | 674 |
| TCR56′ | 675 |
| TCR57′ | 676 |
| TCR58′ | 677 |
| TCR59′ | 678 |
| TCR60′ | 679 |
| TCR61′ | 680 |
| TCR62′ | 681 |
| TCR63′ | 682 |
| TCR64′ | 683 |
| TCR65′ | 684 |
| TCR66′ | 685 |
| TCR67′ | 686 |
| TCR68′ | 687 |
| TCR69′ | 688 |
| TCR70′ | 689 |
| TCR71′ | 690 |

In accordance with a still further embodiment the present invention provides TCR chains or fragments thereof, comprising, in addition to the CDR sequences as defined in Table 5 and optionally a full length TRBV amino acid sequence represented by SEQ-ID Nos. 620 to 690 or a sequence comprising a sequence identity of from 90 to 99.7% to to SEQ-ID Nos. 620 to 690, a full length TRAV amino acid sequence selected from SEQ-ID No. 904 to 974 or an amino acid sequence derived therefrom with a degree of sequence identity in the range of from 90 to 99.7% of SEQ-ID No. 904 to 974 in its alpha chain part in accordance with Table 7 (designated as TCR1″ to TCR71″):

TABLE 7

| TCR No. | SEQ-ID TRAV |
|---|---|
| TCR1″ | 904 |
| TCR2″ | 905 |
| TCR3″ | 906 |
| TCR4″ | 907 |
| TCR5″ | 908 |
| TCR6″ | 909 |
| TCR7″ | 910 |
| TCR8″ | 911 |
| TCR9″ | 912 |
| TCR10″ | 913 |
| TCR11″ | 914 |
| TCR12″ | 915 |
| TCR13″ | 916 |
| TCR14″ | 917 |
| TCR15″ | 918 |
| TCR16″ | 919 |
| TCR17″ | 920 |
| TCR18″ | 921 |
| TCR19″ | 922 |
| TCR20″ | 923 |
| TCR21″ | 924 |
| TCR22″ | 925 |
| TCR23″ | 926 |
| TCR24″ | 927 |
| TCR25″ | 928 |
| TCR26″ | 929 |
| TCR27″ | 930 |
| TCR28″ | 931 |
| TCR29″ | 932 |
| TCR30″ | 933 |
| TCR31″ | 934 |
| TCR32″ | 935 |
| TCR33″ | 936 |
| TCR34″ | 937 |
| TCR35″ | 938 |
| TCR36″ | 939 |
| TCR37″ | 940 |
| TCR38″ | 941 |
| TCR39″ | 942 |
| TCR40″ | 943 |
| TCR41″ | 944 |
| TCR42″ | 945 |
| TCR43″ | 946 |
| TCR44″ | 947 |
| TCR45″ | 948 |
| TCR46″ | 949 |
| TCR47″ | 950 |
| TCR48″ | 951 |
| TCR49″ | 952 |
| TCR50″ | 953 |
| TCR51″ | 954 |
| TCR52″ | 955 |
| TCR53″ | 956 |
| TCR54″ | 957 |
| TCR55″ | 958 |
| TCR56″ | 959 |
| TCR57″ | 960 |
| TCR58″ | 961 |
| TCR59″ | 962 |
| TCR60″ | 963 |
| TCR61″ | 964 |
| TCR62″ | 965 |
| TCR63″ | 966 |
| TCR64″ | 967 |
| TCR65″ | 968 |
| TCR66″ | 969 |
| TCR67″ | 970 |
| TCR68″ | 971 |
| TCR69″ | 972 |
| TCR70″ | 973 |
| TCR71″ | 974 |

In Tables 5 to 7 designations TCR1 to TCR71, TCR1′ to TCR58′ and TCR1″ to BCR58″ are used. In this regard TCR1 represents a TCR chain or a fragment thereof comprising the CDR sequences given in Table 5 for TCR1, TCR1′ represents TCR1 plus the full length sequence given in Table 6 for TCR1′ or a full length sequence having a degree of sequence identity of from 90 to 99.7% to the SEQ-IDs listed in Table 6 and BCR1″ represents BCR1 plus the full length sequence given in Table 7 for BCR1″ or a full length sequence having a degree of sequence identity of from 90 to 99.7% to the SEQ-IDs listed in Table 7. TCR2 to TCR71, TCR2′ to TCR71′and TCR2″ to TCR71″ are to be interpreted accordingly.

The present invention also comprises TCR chains and fragments thereof which cumulatively comprise the CDR sequences in accordance with Table 5, the full length sequences in accordance with Table 6 or full length sequences having a degree of sequence identity of from 90 to 99.7% to the SEQ-IDs listed in Table 6 and the full length sequences in accordance with Table 7 or full length sequences having a degree of sequence identity of from 90 to 99.7% to the SEQ-IDs listed in Table 7. These could be desiganted TCR1″′ to TCR71″′ in an anlalogous manner than explained above.

TCR chains or fragments thereof as defined in Tables 5 to 7 are particularly suitable for use in a method for imaging or diagnosis of atherosclerosis, in particular wherein the method comprises the visualization of atherosclerotic lesions by detection of the TCR chain or fragment thereof previously administered to the patient.

### DETAILED DESCRIPTION

The present invention in an embodiment provides TCR chains or a fragment designated as TCR1 to TCR71 as defined in Table 5, which, in a preferred embodiment also comprise full length sequences as defined in Table 6 and which are designated as TCR1′ to TCR 71′ and in another embodiment also comprise full length sequences as defined in Table 7 are suitable for the diagnosis or therapy of unresolved inflammatory diseases. This in particulart includes TCR chains or fragments thereof comprising an amino acid sequence with a degree of sequence identity in the range of from 90 to 99.7%, preferably of from 92 to 99.7% and particularly preferably of from 95 to 99.7%, of SEQ-ID No. 620 to 690 in its beta chain part and an amino acid sequence with a degree of sequence identity in the range of from 90 to 99.7%, preferably of from 92 to 99.7% and particularly preferably of from 95 to 99.7%, of SEQ-ID No. 904 to 974 in its alpha chain part are also included.

From 3310 TCRs sequences, TCRs designated TCR1 to TCR71 respectively TCR1′ to TCR71′ and TCR1″ to TCR71″ are clonally expanded TCRs, and TCR1 to TCR 71, TCR1′ to TCR71′ and TCR1″ to TCR71″ are presented in diseased mice, but not in wild type mice revealing their unique nature in atherosclerosis.

In the course of the present invention a list of atherosclerosis-relevant monoclonal antibodies and TCR sequences in diseased mice have been found.

Using the monoclonal antibodies, evidence was found that observations made in mice can be translated into human patients with cardiovascular diseases. The monoclonal antibodies and TCRs can be used as a pool of cells from which diagnostic tools in atherosclerosis can be obtained, respectively. The monoclonal antibodies and TCRs also provide a pool to be used to develop therapeutic treatments for atherosclerosis. Moreover, their cognate autoantigens can be used to develop vaccination treatments for atherosclerosis.

It was found that histone 2B antigen and monoclonal antibody against histone 2B are involved in atherosclerosis in mouse models and in patients with cardiovascular diseases. This proof of concept evidence showed that ATLO-derived monoclonal antibodies and their cognate antigens form a pool of sequences to be developed as serum biomarkers to diagnose and possibly predict atherosclerosis.

It was also found that several ATLO-derived monoclonal antibodies recognize atherosclerosis plaque constituents. These data provide imaging tools for atherosclerosis. They also provide a pool of sequences to develop treatment regimens to attenuate atherosclerosis plaques.

The monoclonal antibodies (encoded in their BCRs) found provide tools to deliver drugs to atherosclerotic plaques environments and therefore can be applied to treat atherosclerosis.

Histone 2B was identified as cognate antigen for at least one of ATLO-derived antibodies (BCR56, BCR56′, BCR56″ and BCR56′″). Thus Histone 2B and cognate antigens of other ATLO-derived antibodies can be used to design novel atherosclerosis vaccination strategies. Each of the autoantigens provide powerful tools to vaccinate humans to prevent and or treat patients with advanced disease providing causal treatment options not available until today as atherosclrosis remains a largely untreatable unresolvable inflammatory disease of arteries.

The present invention defines clonally expanded T cells in atherosclerotic plaques and in ATLOs and identified their TCR sequences. These clonally expanded TCRs can be used to design novel T cell-based diagnosis tools and T cell-based immune therapies.

The transcriptome analyses reported here show that there is a breakdown of tolerance in atherosclerotic mice providing tools to develop treatments to recover tolerance in atherosclerotic patients as is presently used in cancer immunotherapies.

The data show that atherosclerosis-relevant B2 autoimmune responses are organized in secondary lymphoid organs and ATLOs in aged ApoE^{-/-} mice. Using single cell sequences, B cell responses were compared between secondary lymphoid organs and ATLOs and provide evidence that ATLO is a functionally active lymphoid organ that may organize atherosclerosis-relevant B2 autoimmune responses. Using single cell cloning and recombinant antibody production technologies, it was observed that several ATLO germinal center-derived antibodies recognized atherosclerotic plaque constituents. These data show that ATLOs organize atherosclerosis-relevant autoimmune B2 responses. Using ATLO germinal center-derived BCR56, BCR56′, BCR56″ and BCR56″′ antibody histone 2B (H2B) protein was identified as the cognate antigen for BCR56, BCR56′, BCR56″ and BCR56″′ antibody. Importantly, the same H2B protein also been observed in early stages of atherosclerosis in mice, as well as in human atherosclerotic plaques. We identified a series of monoclonal antibodies and TCR sequences in diseased mice. These monoclonal antibodies and TCRs and their cognate antigens can be used as novel diagnosis tools and treatments for atherosclerosis.

The BCR chains or fragments thereof and TCR chains or fragments thereof in accordance with the present invention can advantageously be used in a method for in vivo atherosclerotic lesions in a patient, preferably in a method for in vivo diagnosis of atherosclerosis. In a preferred embodiment such method comprises the visualization of atherosclerotic lesions by detection of the BCR chain or fragment thereof or the TCR chain or fragment thereof previously administered to the patient.

The invention also comprises pharmaceutical compositions comprising any ogfg the BCR chains or fragments thereof or the TCR chains or fragments thereof and pharmaceutically acceptable excipients and carriers.

In another embodiment the invention refers to a method for treating atherosclerosis which comprises the administration of a therapeutically effective dose of any of the BCR chains or fragments thereof or TCR chains or fragments thereof in accordance with the present invention.

Therapeutically effective dose for the purpose of the present invention is intended to denote an amount which, when administered to the patient creates a positive therapeutic response in a subject having atherosclerosis.

Finally, the present invention also encompasses a kit adopted for the diagnosis of atheroclerosis comprising 1) any of the BCR chains or fragments thereof or TCR chains of fragments thereof in accordance with the present invention and 2) reagents or means for the detection or visualization of any of the BCR chains or fragments thereof or TCR chains of fragments thereof in accordance with the present invention.

The BCR chains or fragments thereof or TCR chains or fragments thereof in accordance with the present invention may e.g. be labelled with a radioactive tracer or a contrast agent and the visualization can then be achieved by nuclear imaging or magneitc resonance imaging.

1. A B-cell receptor (BCR) chains or fragments thereof comprising:
a heavy chain variable regions (IgH) and light chain chain variable regions (IgL) wherein said heavy chain variable regions (IgH) comprise complementary determining regions IgH CDR1, IgH CDR2 and IgH CDR3 and wherein said light chain variable regions comprise complimentary determining regions IgL CDR1 and IgL CDR3 wherein IgH CDR1 comprises amino acid sequences selected from SEQ-ID Nos. 1 to 58, IgH CDR2 comprises sequences selected from SEQ-ID Nos. 59 to 117, IgH CDR3 comprises sequences selected from SEQ-ID Nos. 117 to 174, IgL CDR1 comprises sequences selected from SEQ-ID Nos. 233 to 290, and IgL CDR3 comprises sequences selected from SEQ-ID Nos. 291 to 348 in accordance with Table 1:
TABLE 1

| | SEQ-ID | SEQ-ID | SEQ-ID | SEQ-ID | SEQ-ID |
|---|---|---|---|---|---|
| BCR | IgH | IgH | IgH | IgL | IgL |
| No. | CDR1 | CDR2 | CDR3 | CDR1 | CDR3 |
| BCR1 | 1 | 59 | 117 | 233 | 291 |
| BCR2 | 2 | 60 | 118 | 234 | 292 |
| BCR3 | 3 | 61 | 119 | 235 | 293 |
| BCR4 | 4 | 62 | 120 | 236 | 294 |
| BCR5 | 5 | 63 | 121 | 237 | 295 |
| BCR6 | 6 | 64 | 122 | 238 | 296 |
| BCR7 | 7 | 65 | 123 | 239 | 297 |
| BCR8 | 8 | 66 | 124 | 240 | 298 |
| BCR9 | 9 | 67 | 125 | 241 | 299 |
| BCR10 | 10 | 68 | 126 | 242 | 300 |
| BCR11 | 11 | 69 | 127 | 243 | 301 |
| BCR12 | 12 | 70 | 128 | 244 | 302 |
| BCR13 | 13 | 71 | 129 | 245 | 303 |
| BCR14 | 14 | 72 | 130 | 246 | 304 |
| BCR15 | 15 | 73 | 131 | 247 | 305 |
| BCR16 | 16 | 74 | 132 | 248 | 306 |
| BCR17 | 17 | 75 | 133 | 249 | 307 |
| BCR18 | 18 | 76 | 134 | 250 | 308 |
| BCR19 | 19 | 77 | 135 | 251 | 309 |
| BCR20 | 20 | 78 | 136 | 252 | 310 |
| BCR21 | 21 | 79 | 137 | 253 | 311 |
| BCR22 | 22 | 80 | 138 | 254 | 312 |
| BCR23 | 23 | 81 | 139 | 255 | 313 |
| BCR24 | 24 | 82 | 140 | 256 | 314 |
| BCR25 | 25 | 83 | 141 | 257 | 315 |
| BCR26 | 26 | 84 | 142 | 258 | 316 |
| BCR27 | 27 | 85 | 143 | 259 | 317 |
| BCR28 | 28 | 86 | 144 | 260 | 318 |
| BCR29 | 29 | 87 | 145 | 261 | 319 |
| BCR30 | 30 | 88 | 146 | 262 | 320 |
| BCR31 | 31 | 89 | 147 | 263 | 321 |
| BCR32 | 32 | 90 | 148 | 264 | 322 |
| BCR33 | 33 | 91 | 149 | 265 | 323 |
| BCR34 | 34 | 92 | 150 | 266 | 324 |
| BCR35 | 35 | 93 | 151 | 267 | 325 |
| BCR36 | 36 | 94 | 152 | 268 | 326 |
| BCR37 | 37 | 95 | 153 | 269 | 327 |
| BCR38 | 38 | 96 | 154 | 270 | 328 |
| BCR39 | 39 | 97 | 155 | 271 | 329 |
| BCR40 | 40 | 98 | 156 | 272 | 330 |
| BCR41 | 41 | 99 | 157 | 273 | 331 |
| BCR42 | 42 | 100 | 158 | 274 | 332 |
| BCR43 | 43 | 101 | 159 | 275 | 333 |
| BCR44 | 44 | 102 | 160 | 276 | 334 |
| BCR45 | 45 | 103 | 161 | 277 | 335 |
| BCR46 | 46 | 104 | 162 | 278 | 336 |
| BCR47 | 47 | 105 | 163 | 279 | 337 |
| BCR48 | 48 | 106 | 164 | 280 | 338 |
| BCR49 | 49 | 107 | 165 | 281 | 339 |
| BCR50 | 50 | 108 | 166 | 282 | 340 |
| BCR51 | 51 | 109 | 167 | 283 | 341 |
| BCR52 | 52 | 110 | 168 | 284 | 342 |
| BCR53 | 53 | 111 | 169 | 285 | 343 |
| BCR54 | 54 | 112 | 170 | 286 | 344 |
| BCR55 | 55 | 113 | 171 | 287 | 345 |
| BCR56 | 56 | 114 | 172 | 288 | 346 |
| BCR57 | 57 | 115 | 173 | 289 | 347 |
| BCR58 | 58 | 116 | 174 | 290 | 348. |

2. The BCR chain or fragment thereof of claim 1 further comprising IgL Full Length amino acid sequences selected from SEQ-ID Nos. 175 to 232 or amino acid full length sequences derived therefrom with a degree of sequence identity in the range of from 90 to 99.7% to SEQ-ID Nos. 175 to 232 in accordance with Table 2:
TABLE 2

| BCR No. | SEQ-ID IgH Full length |
|---|---|
| BCR1′ | 175 |
| BCR2′ | 176 |
| BCR3′ | 177 |
| BCR4′ | 178 |
| BCR5′ | 179 |
| BCR6′ | 180 |
| BCR7′ | 181 |
| BCR8′ | 182 |
| BCR9′ | 183 |
| BCR10′ | 184 |
| BCR11′ | 185 |
| BCR12′ | 186 |
| BCR13′ | 187 |
| BCR14′ | 188 |
| BCR15′ | 189 |
| BCR16′ | 190 |
| BCR17′ | 191 |
| BCR18′ | 192 |
| BCR19′ | 193 |
| BCR20′ | 194 |
| BCR21′ | 195 |
| BCR22′ | 196 |
| BCR23′ | 197 |
| BCR24′ | 198 |
| BCR25′ | 199 |
| BCR26′ | 200 |
| BCR27′ | 201 |
| BCR28′ | 202 |
| BCR29′ | 203 |
| BCR30′ | 204 |
| BCR31′ | 205 |
| BCR32′ | 206 |
| BCR33′ | 207 |
| BCR34′ | 208 |
| BCR35′ | 209 |
| BCR36′ | 210 |
| BCR37′ | 211 |
| BCR38′ | 212 |
| BCR39′ | 213 |
| BCR40′ | 214 |
| BCR41′ | 215 |
| BCR42′ | 216 |
| BCR43′ | 217 |
| BCR44′ | 218 |
| BCR45′ | 219 |
| BCR46′ | 220 |
| BCR47′ | 221 |
| BCR48′ | 222 |
| BCR49′ | 223 |
| BCR50′ | 224 |
| BCR51′ | 225 |
| BCR52′ | 226 |
| BCR53′ | 227 |
| BCR54′ | 228 |
| BCR55′ | 229 |
| BCR56′ | 230 |
| BCR57′ | 231 |
| BCR58′ | 232. |

3. The BCR chain or fragment thereof of claim 2, further comprising an IgL Full Length amino acid sequence selected from SEQ-ID Nos. 349 to 406 or an IgL full length amino acid sequence derived from SEQ-ID Nos. 349 to 406 with a degree of sequence identity in the range of from 90 to 99.7% in accordance with Table 3
TABLE 3

| BCR No. | SEQ-ID IgL Full Length |
|---|---|
| BCR1″ | 349 |
| BCR2″ | 350 |
| BCR3″ | 351 |
| BCR4″ | 352 |
| BCR5″ | 353 |
| BCR6″ | 354 |
| BCR7″ | 355 |
| BCR8″ | 356 |
| BCR9″ | 357 |
| BCR10″ | 358 |
| BCR11″ | 359 |
| BCR12″ | 360 |
| BCR13″ | 361 |
| BCR14″ | 362 |
| BCR15″ | 363 |
| BCR16″ | 364 |
| BCR17″ | 365 |
| BCR18″ | 366 |
| BCR19″ | 367 |
| BCR20″ | 368 |
| BCR21″ | 369 |
| BCR22″ | 370 |
| BCR23″ | 371 |
| BCR24″ | 372 |
| BCR25″ | 373 |
| BCR26″ | 374 |
| BCR27″ | 375 |
| BCR28″ | 376 |
| BCR29″ | 377 |
| BCR30″ | 378 |
| BCR31″ | 379 |
| BCR32″ | 380 |
| BCR33″ | 381 |
| BCR34″ | 382 |
| BCR35″ | 383 |
| BCR36″ | 384 |
| BCR37″ | 385 |
| BCR38″ | 386 |
| BCR39″ | 387 |
| BCR40″ | 388 |
| BCR41″ | 389 |
| BCR42″ | 390 |
| BCR43″ | 391 |
| BCR44″ | 392 |
| BCR45″ | 393 |
| BCR46″ | 394 |
| BCR47″ | 395 |
| BCR48″ | 396 |
| BCR49″ | 397 |
| BCR50″ | 398 |
| BCR51″ | 399 |
| BCR52″ | 400 |
| BCR53″ | 401 |
| BCR54″ | 402 |
| BCR55″ | 403 |
| BCR56″ | 404 |
| BCR57″ | 405 |
| BCR58″ | 406. |

4. The BCR chain or fragment thereof of claim 3, further comprising an IgL full length amino acid sequence selected from SEQ-ID Nos. 175 to 232 or amino acid full length sequences derived therefrom with a degree of sequence identity in the range of from 90 to 99.7% to SEQ-ID Nos. 175 to 232 in accordance with Table 2 and an IgL Full Length amino acid sequence selected from SEQ-ID Nos. 349 to 406 or full length amino acid sequences derived from SEQ-ID Nos. 349 to 406 with a degree of sequence identity in the range of from 90 to 99.7% in accordance with Table 3.

5. The BCR chain or fragment thereof of claim 4, wherein said-light chain variable regions (IgL) comprise sequences of three amino acids in complementary determining region IgL CDR2 as defined in Table 4.
TABLE 4

| BCR No. | Amino acid sequence IgL CDR2 |
|---|---|
| BCR1″′ | YAS |
| BCR2″′ | EGN |
| BCR3″′ | RAS |
| BCR4″′ | LAS |
| BCR5″′ | RAS |
| BCR6″′ | AAS |
| BCR7″′ | AAS |
| BCR8″′ | AAS |
| BCR9″′ | YAS |
| BCR10″′ | AAS |
| BCR11″′ | AAS |
| BCR12″′ | AAS |
| BCR13″′ | RAS |
| BCR14″′ | AAS |
| BCR15″′ | GTS |
| BCR16″′ | AAS |
| BCR17″′ | AAS |
| BCR18″′ | AAS |
| BCR19″′ | AAS |
| BCR20″′ | YTS |
| BCR21″′ | STS |
| BCR22″′ | RAS |
| BCR23″′ | AAS |
| BCR24″′ | RAS |
| BCR25″′ | GTN |
| BCR26″′ | GAS |
| BCR27″′ | STS |
| BCR28″′ | YAS |
| BCR29″′ | YAS |
| BCR30″′ | AAS |
| BCR31″′ | KAS |
| BCR32″′ | AAS |
| BCR33″′ | STS |
| BCR34″′ | AAS |
| BCR35″′ | AAS |
| BCR36″′ | RAS |
| BCR37″′ | STS |
| BCR38″′ | YTS |
| BCR39″′ | AAS |
| BCR40″′ | AAS |
| BCR41″′ | STS |
| BCR42″′ | AAS |
| BCR43″′ | IAS |
| BCR44″′ | YTS |
| BCR45″′ | KAS |
| BCR46″′ | NTS |
| BCR47″′ | VAS |
| BCR48″′ | YTS |
| BCR49″′ | YTS |
| BCR50″′ | RTS |
| BCR51″′ | RTS |
| BCR52″′ | YAS |
| BCR53″′ | YTF |
| BCR54″′ | YTS |
| BCR55″′ | IAS |
| BCR56″′ | RAN |
| BCR57″′ | YTS |
| BCR58″′ | YTS. |

6. The BCR chain or fragment thereof of claim 4, for use in diagnosis and/or therapy of atherosclerosis.

7. The BCR chain or fragment thereof of claim 6, wherein using at least one of BCR56, BCR56′, BCR56″ or BCR56″′ for the therapy of atherosclerosis.

8. The BCR chain or fragment thereof of claim 6 for use in a method for imaging or diagnosis of atherosclerosis wherein the method comprises the visualization of atherosclerotic lesions by detection of the BCR chain or fragment thereof previously administered to the patient.

9. A T-cell receptor (TCR) chain or part thereof, comprising:
a beta chain part (TRBV) and an alpha chain part (TRAV) wherein said beta chain part (TRBV) comprises complementary determining regions TRBV CDR1, TRBV CDR2 and TRBV CDR3 and wherein said alpha chain part TRAV comprises complimentary determining regions TRAV CDR1, TRAV CDR2 and TRAV CDR3 wherein TRBV CDR1 comprises amino acid sequences selected from SEQ-ID Nos. 407 to 477, TRBV CDR2 comprises sequences selected from SEQ-ID Nos. 478 to 548, TRBV CDR3 comprises sequences selected from SEQ-ID Nos.549 to 619, TRAV CDR1 comprises sequences selected from SEQ-ID Nos. 691 to 761, TRAV CDR2 comprises sequences selected from SEQ-ID Nos. 762 to 832 and TRAV CDR3 comprises sequences selected from SEQ-IDs No. 833 to 903 in accordance with Table 5:
TABLE 5

| | SEQ-ID | SEQ-ID | SEQ-ID | SEQ-ID | SEQ-ID | SEQ-ID |
|---|---|---|---|---|---|---|
| TCR | TRBV | TRBV | TRBV | TRAV | TRAV | TRAV |
| No. | CDR1 | CDR2 | CDR3 | CDR1 | CDR2 | CDR3 |
| TCR1 | 407 | 478 | 549 | 691 | 762 | 833 |
| TCR2 | 408 | 479 | 550 | 692 | 763 | 834 |
| TCR3 | 409 | 480 | 551 | 693 | 764 | 835 |
| TCR4 | 410 | 481 | 552 | 694 | 765 | 836 |
| TCR5 | 411 | 482 | 553 | 695 | 766 | 837 |
| TCR6 | 412 | 483 | 554 | 696 | 767 | 838 |
| TCR7 | 413 | 484 | 555 | 697 | 768 | 839 |
| TCR8 | 414 | 485 | 556 | 698 | 769 | 840 |
| TCR9 | 415 | 486 | 557 | 699 | 770 | 841 |
| TCR10 | 416 | 487 | 558 | 700 | 771 | 842 |
| TCR11 | 417 | 488 | 559 | 701 | 772 | 843 |
| TCR12 | 418 | 489 | 560 | 702 | 773 | 844 |
| TCR13 | 419 | 490 | 561 | 703 | 774 | 845 |
| TCR14 | 420 | 491 | 562 | 704 | 775 | 846 |
| TCR15 | 421 | 492 | 563 | 705 | 776 | 847 |
| TCR16 | 422 | 493 | 564 | 706 | 777 | 848 |
| TCR17 | 423 | 494 | 565 | 707 | 778 | 849 |
| TCR18 | 424 | 495 | 566 | 708 | 779 | 850 |
| TCR19 | 425 | 496 | 567 | 709 | 780 | 851 |
| TCR20 | 426 | 497 | 568 | 710 | 781 | 852 |
| TCR21 | 427 | 498 | 569 | 711 | 782 | 853 |
| TCR22 | 428 | 499 | 570 | 712 | 783 | 854 |
| TCR23 | 429 | 500 | 571 | 713 | 784 | 855 |
| TCR24 | 430 | 501 | 572 | 714 | 785 | 856 |
| TCR25 | 431 | 502 | 573 | 715 | 786 | 857 |
| TCR26 | 432 | 503 | 574 | 716 | 787 | 858 |
| TCR27 | 433 | 504 | 575 | 717 | 788 | 859 |
| TCR28 | 434 | 505 | 576 | 718 | 789 | 860 |
| TCR29 | 435 | 506 | 577 | 719 | 790 | 861 |
| TCR30 | 436 | 507 | 578 | 720 | 791 | 862 |
| TCR31 | 437 | 508 | 579 | 721 | 792 | 863 |
| TCR32 | 438 | 509 | 580 | 722 | 793 | 864 |
| TCR33 | 439 | 510 | 581 | 723 | 794 | 865 |
| TCR34 | 440 | 511 | 582 | 724 | 795 | 866 |
| TCR35 | 441 | 512 | 583 | 725 | 796 | 867 |
| TCR36 | 442 | 513 | 584 | 726 | 797 | 868 |
| TCR37 | 443 | 514 | 585 | 727 | 798 | 869 |
| TCR38 | 444 | 515 | 586 | 728 | 799 | 870 |
| TCR39 | 445 | 516 | 587 | 729 | 800 | 871 |
| TCR40 | 446 | 517 | 588 | 730 | 801 | 872 |
| TCR41 | 447 | 518 | 589 | 731 | 802 | 873 |
| TCR42 | 448 | 519 | 590 | 732 | 803 | 874 |
| TCR43 | 449 | 520 | 591 | 733 | 804 | 875 |
| TCR44 | 450 | 521 | 592 | 734 | 805 | 876 |
| TCR45 | 451 | 522 | 593 | 735 | 806 | 877 |
| TCR46 | 452 | 523 | 594 | 736 | 807 | 878 |
| TCR47 | 453 | 524 | 595 | 737 | 808 | 879 |
| TCR48 | 454 | 525 | 596 | 738 | 809 | 880 |
| TCR49 | 455 | 526 | 597 | 739 | 810 | 881 |
| TCR50 | 456 | 527 | 598 | 740 | 811 | 882 |
| TCR51 | 457 | 528 | 599 | 741 | 812 | 883 |
| TCR52 | 458 | 529 | 600 | 742 | 813 | 884 |
| TCR53 | 459 | 530 | 601 | 743 | 814 | 885 |
| TCR54 | 460 | 531 | 602 | 744 | 815 | 886 |
| TCR55 | 461 | 532 | 603 | 745 | 816 | 887 |
| TCR56 | 462 | 533 | 604 | 746 | 817 | 888 |
| TCR57 | 463 | 534 | 605 | 747 | 818 | 889 |
| TCR58 | 464 | 535 | 606 | 748 | 819 | 890 |
| TCR59 | 465 | 536 | 607 | 749 | 820 | 891 |
| TCR60 | 466 | 537 | 608 | 750 | 821 | 892 |
| TCR61 | 467 | 538 | 609 | 751 | 822 | 893 |
| TCR62 | 468 | 539 | 610 | 752 | 823 | 894 |
| TCR63 | 469 | 540 | 611 | 753 | 824 | 895 |
| TCR64 | 470 | 541 | 612 | 754 | 825 | 896 |
| TCR65 | 471 | 542 | 613 | 755 | 826 | 897 |
| TCR66 | 472 | 543 | 614 | 756 | 827 | 898 |
| TCR67 | 473 | 544 | 615 | 757 | 828 | 899 |
| TCR68 | 474 | 545 | 616 | 758 | 829 | 900 |
| TCR69 | 475 | 546 | 617 | 759 | 830 | 901 |
| TCR70 | 476 | 547 | 618 | 760 | 831 | 902 |
| TCR71 | 477 | 548 | 619 | 761 | 832 | 903 |

10. The TCR chain or fragment thereof of claim 9, further comprising a full length TRBV amino acid sequence selected from SEQ-ID Nos. 620 to 690 or an amino acid sequence derived therefrom with a degree of sequence identity in the range of from 90 to 99.7% to SEQ-ID No. 620 to 690 in its beta chain part in accordance with Table 6:
TABLE 6

| TCR No. | SEQ-ID TRBV |
|---|---|
| TCR1′ | 620 |
| TCR2′ | 621 |
| TCR3′ | 622 |
| TCR4′ | 623 |
| TCR5′ | 624 |
| TCR6′ | 625 |
| TCR7′ | 626 |
| TCR8′ | 627 |
| TCR9′ | 628 |
| TCR10′ | 629 |
| TCR11′ | 630 |
| TCR12′ | 631 |
| TCR13′ | 632 |
| TCR14′ | 633 |
| TCR15′ | 634 |
| TCR16′ | 635 |
| TCR17′ | 636 |
| TCR18′ | 637 |
| TCR19′ | 638 |
| TCR20′ | 639 |
| TCR21′ | 640 |
| TCR22′ | 641 |
| TCR23′ | 642 |
| TCR24′ | 643 |
| TCR25′ | 644 |
| TCR26′ | 645 |
| TCR27′ | 646 |
| TCR28′ | 647 |
| TCR29′ | 648 |
| TCR30′ | 649 |
| TCR31′ | 650 |
| TCR32′ | 651 |
| TCR33′ | 652 |
| TCR34′ | 653 |
| TCR35′ | 654 |
| TCR36′ | 655 |
| TCR37′ | 656 |
| TCR38′ | 657 |
| TCR39′ | 658 |
| TCR40′ | 659 |
| TCR41′ | 660 |
| TCR42′ | 661 |
| TCR43′ | 662 |
| TCR44′ | 663 |
| TCR45′ | 664 |
| TCR46′ | 665 |
| TCR47′ | 666 |
| TCR48′ | 667 |
| TCR49′ | 668 |
| TCR50′ | 669 |
| TCR51′ | 670 |
| TCR52′ | 671 |
| TCR53′ | 672 |
| TCR54′ | 673 |
| TCR55′ | 674 |
| TCR56′ | 675 |
| TCR57′ | 676 |
| TCR58′ | 677 |
| TCR59′ | 678 |
| TCR60′ | 679 |
| TCR61′ | 680 |
| TCR62′ | 681 |
| TCR63′ | 682 |
| TCR64′ | 683 |
| TCR65′ | 684 |
| TCR66′ | 685 |
| TCR67′ | 686 |
| TCR68′ | 687 |
| TCR69′ | 688 |
| TCR70′ | 689 |
| TCR71′ | 690. |

11. The TCR chain or fragment thereof of claim 9, comprising a full length TRAV amino acid sequence selected from SEQ-ID No. 904 to 974 or an amino acid sequence derived therefrom with a degree of sequence identity in the range of from 90 to 99.7% of SEQ-ID No. 904 to 974 in its alpha chain part in accordance with Table 7:
TABLE 7

| TCR No. | SEQ-ID TRAV |
|---|---|
| TCR1″ | 904 |
| TCR2″ | 905 |
| TCR3″ | 906 |
| TCR4″ | 907 |
| TCR5″ | 908 |
| TCR6″ | 909 |
| TCR7″ | 910 |
| TCR8″ | 911 |
| TCR9″ | 912 |
| TCR10″ | 913 |
| TCR11″ | 914 |
| TCR12″ | 915 |
| TCR13″ | 916 |
| TCR14″ | 917 |
| TCR15″ | 918 |
| TCR16″ | 919 |
| TCR17″ | 920 |
| TCR18″ | 921 |
| TCR19″ | 922 |
| TCR20″ | 923 |
| TCR21″ | 924 |
| TCR22″ | 925 |
| TCR23″ | 926 |
| TCR24″ | 927 |
| TCR25″ | 928 |
| TCR26″ | 929 |
| TCR27″ | 930 |
| TCR28″ | 931 |
| TCR29″ | 932 |
| TCR30″ | 933 |
| TCR31″ | 934 |
| TCR32″ | 935 |
| TCR33″ | 936 |
| TCR34″ | 937 |
| TCR35″ | 938 |
| TCR36″ | 939 |
| TCR37″ | 940 |
| TCR38″ | 941 |
| TCR39″ | 942 |
| TCR40″ | 943 |
| TCR41″ | 944 |
| TCR42″ | 945 |
| TCR43″ | 946 |
| TCR44″ | 947 |
| TCR45″ | 948 |
| TCR46″ | 949 |
| TCR47″ | 950 |
| TCR48″ | 951 |
| TCR49″ | 952 |
| TCR50″ | 953 |
| TCR51″ | 954 |
| TCR52″ | 955 |
| TCR53″ | 956 |
| TCR54″ | 957 |
| TCR55″ | 958 |
| TCR56″ | 959 |
| TCR57″ | 960 |
| TCR58″ | 961 |
| TCR59″ | 962 |
| TCR60″ | 963 |
| TCR61″ | 964 |
| TCR62″ | 965 |
| TCR63″ | 966 |
| TCR64″ | 967 |
| TCR65″ | 968 |
| TCR66″ | 969 |
| TCR67″ | 970 |
| TCR68″ | 971 |
| TCR69″ | 972 |
| TCR70″ | 973 |
| TCR71″ | 974. |

12. The TCR chain or fragment thereof of claim 9, further comprising a full length TRBV amino acid sequence selected from SEQ-ID Nos. 620 to 690 or an amino acid sequence derived therefrom with a degree of sequence identity in the range of from 90 to 99.7% of SEQ-ID No. 620 to 690 in its beta chain part and a full length TRAV amino acid sequence selected from SEQ-ID No. 904 to 974 or an amino acid sequence derived therefrom with a degree of sequence identity in the range of from 90 to 99.7% of SEQ-ID No. 904 to 974 in its alpha chain part.

13. The TCR chain or fragment thereof of claim 12 for use in diagnosis and/or therapy of atherosclerosis.

14. The TCR chain or fragment thereof of claim 12 for use in a method for imaging or diagnosis of atherosclerosis wherein the method comprises the visualization of atherosclerotic lesions by detection of the TCR chain or fragment thereof previously administered to the patient.


SEQUENCE LISTING (974 sequences)

SEQ ID NO: 1 - protein, 8 aa, Mus musculus
  >SEQUENCE_0001 BCR001 IgH CDR1 Cell_001 Mus_musculus
GYAFSSSW

SEQ ID NO: 2 - protein, 8 aa, Mus musculus
  >SEQUENCE_0002 BCR002 IgH CDR1 Cell_002 Mus_musculus
GYAFSSSW

SEQ ID NO: 3 - protein, 8 aa, Mus musculus
  >SEQUENCE_0003 BCR003 IgH CDR1 Cell_003 Mus_musculus
GYAFSSSW

SEQ ID NO: 4 - protein, 8 aa, Mus musculus
  >SEQUENCE_0004 BCR004 IgH CDR1 Cell_004 Mus_musculus
GYTFTSYW

SEQ ID NO: 5 - protein, 8 aa, Mus musculus
  >SEQUENCE_0005 BCR005 IgH CDR1 Cell_005 Mus_musculus
GYSFTGYY

SEQ ID NO: 6 - protein, 8 aa, Mus musculus
  >SEQUENCE_0006 BCR006 IgH CDR1 Cell_006 Mus_musculus
GYSFTGYY

SEQ ID NO: 7 - protein, 8 aa, Mus musculus
  >SEQUENCE_0007 BCR007 IgH CDR1 Cell_007 Mus_musculus
GYTFTSSW

SEQ ID NO: 8 - protein, 8 aa, Mus musculus
  >SEQUENCE_0008 BCR008 IgH CDR1 Cell_008 Mus_musculus
GYTFTSYW

SEQ ID NO: 9 - protein, 8 aa, Mus musculus
  >SEQUENCE_0009 BCR009 IgH CDR1 Cell_009 Mus_musculus
GFSLTNYG

SEQ ID NO: 10 - protein, 8 aa, Mus musculus
  >SEQUENCE_0010 BCR010 IgH CDR1 Cell_010 Mus_musculus
GFTFTDYY

SEQ ID NO: 11 - protein, 8 aa, Mus musculus
  >SEQUENCE_0011 BCR011 IgH CDR1 Cell_011 Mus_musculus
GYTFTDYY

SEQ ID NO: 12 - protein, 8 aa, Mus musculus
  >SEQUENCE_0012 BCR012 IgH CDR1 Cell_012 Mus_musculus
GYTFTSYW

SEQ ID NO: 13 - protein, 8 aa, Mus musculus
  >SEQUENCE_0013 BCR013 IgH CDR1 Cell_013 Mus_musculus
GYTFTSYW

SEQ ID NO: 14 - protein, 8 aa, Mus musculus
  >SEQUENCE_0014 BCR014 IgH CDR1 Cell_014 Mus_musculus
GFTFTDYY

SEQ ID NO: 15 - protein, 8 aa, Mus musculus
  >SEQUENCE_0015 BCR015 IgH CDR1 Cell_015 Mus_musculus
GYAFSSSW

SEQ ID NO: 16 - protein, 8 aa, Mus musculus
  >SEQUENCE_0016 BCR016 IgH CDR1 Cell_016 Mus_musculus
GYSFTAYY

SEQ ID NO: 17 - protein, 8 aa, Mus musculus
  >SEQUENCE_0017 BCR017 IgH CDR1 Cell_017 Mus_musculus
GYSFTGYY

SEQ ID NO: 18 - protein, 8 aa, Mus musculus
  >SEQUENCE_0018 BCR018 IgH CDR1 Cell_018 Mus_musculus
GYSFTGYF

SEQ ID NO: 19 - protein, 8 aa, Mus musculus
  >SEQUENCE_0019 BCR019 IgH CDR1 Cell_019 Mus_musculus
GYTFTDYY

SEQ ID NO: 20 - protein, 8 aa, Mus musculus
  >SEQUENCE_0020 BCR020 IgH CDR1 Cell_020 Mus_musculus
GYAFSNYW

SEQ ID NO: 21 - protein, 8 aa, Mus musculus
  >SEQUENCE_0021 BCR021 IgH CDR1 Cell_021 Mus_musculus
GYSFSGYY

SEQ ID NO: 22 - protein, 8 aa, Mus musculus
  >SEQUENCE_0022 BCR022 IgH CDR1 Cell_022 Mus_musculus
GYTFTNYT

SEQ ID NO: 23 - protein, 8 aa, Mus musculus
  >SEQUENCE_0023 BCR023 IgH CDR1 Cell_023 Mus_musculus
GYTFTSYW

SEQ ID NO: 24 - protein, 8 aa, Mus musculus
  >SEQUENCE_0024 BCR024 IgH CDR1 Cell_024 Mus_musculus
GYSFTGYY

SEQ ID NO: 25 - protein, 8 aa, Mus musculus
  >SEQUENCE_0025 BCR025 IgH CDR1 Cell_025 Mus_musculus
GFSLTSYG

SEQ ID NO: 26 - protein, 8 aa, Mus musculus
  >SEQUENCE_0026 BCR026 IgH CDR1 Cell_026 Mus_musculus
GSTFTSYW

SEQ ID NO: 27 - protein, 8 aa, Mus musculus
  >SEQUENCE_0027 BCR027 IgH CDR1 Cell_027 Mus_musculus
GYTFTDYY

SEQ ID NO: 28 - protein, 8 aa, Mus musculus
  >SEQUENCE_0028 BCR028 IgH CDR1 Cell_028 Mus_musculus
GYSFTAYY

SEQ ID NO: 29 - protein, 8 aa, Mus musculus
  >SEQUENCE_0029 BCR029 IgH CDR1 Cell_029 Mus_musculus
GYTFTSYW

SEQ ID NO: 30 - protein, 8 aa, Mus musculus
  >SEQUENCE_0030 BCR030 IgH CDR1 Cell_030 Mus_musculus
GYTFTNYT

SEQ ID NO: 31 - protein, 8 aa, Mus musculus
  >SEQUENCE_0031 BCR031 IgH CDR1 Cell_031 Mus_musculus
GYTFTDYY

SEQ ID NO: 32 - protein, 8 aa, Mus musculus
  >SEQUENCE_0032 BCR032 IgH CDR1 Cell_032 Mus_musculus
GYTFTSYW

SEQ ID NO: 33 - protein, 8 aa, Mus musculus
  >SEQUENCE_0033 BCR033 IgH CDR1 Cell_033 Mus_musculus
GYTFTDYY

SEQ ID NO: 34 - protein, 8 aa, Mus musculus
  >SEQUENCE_0034 BCR034 IgH CDR1 Cell_034 Mus_musculus
GFTFSDYG

SEQ ID NO: 35 - protein, 8 aa, Mus musculus
  >SEQUENCE_0035 BCR035 IgH CDR1 Cell_035 Mus_musculus
GYSFIDYN

SEQ ID NO: 36 - protein, 8 aa, Mus musculus
  >SEQUENCE_0036 BCR036 IgH CDR1 Cell_036 Mus_musculus
GYTFTDYY

SEQ ID NO: 37 - protein, 8 aa, Mus musculus
  >SEQUENCE_0037 BCR037 IgH CDR1 Cell_037 Mus_musculus
DYAFSNYW

SEQ ID NO: 38 - protein, 8 aa, Mus musculus
  >SEQUENCE_0038 BCR038 IgH CDR1 Cell_038 Mus_musculus
GFNIKDDY

SEQ ID NO: 39 - protein, 8 aa, Mus musculus
  >SEQUENCE_0039 BCR039 IgH CDR1 Cell_039 Mus_musculus
GYTFTDYY

SEQ ID NO: 40 - protein, 8 aa, Mus musculus
  >SEQUENCE_0040 BCR040 IgH CDR1 Cell_040 Mus_musculus
GYIFTSYW

SEQ ID NO: 41 - protein, 8 aa, Mus musculus
  >SEQUENCE_0041 BCR041 IgH CDR1 Cell_041 Mus_musculus
GHTFTSHW

SEQ ID NO: 42 - protein, 8 aa, Mus musculus
  >SEQUENCE_0042 BCR042 IgH CDR1 Cell_042 Mus_musculus
GYTFTDYY

SEQ ID NO: 43 - protein, 8 aa, Mus musculus
  >SEQUENCE_0043 BCR043 IgH CDR1 Cell_043 Mus_musculus
GYTFTSYW

SEQ ID NO: 44 - protein, 8 aa, Mus musculus
  >SEQUENCE_0044 BCR044 IgH CDR1 Cell_044 Mus_musculus
GYTFTSYW

SEQ ID NO: 45 - protein, 8 aa, Mus musculus
  >SEQUENCE_0045 BCR045 IgH CDR1 Cell_045 Mus_musculus
GYTFTDYY

SEQ ID NO: 46 - protein, 8 aa, Mus musculus
  >SEQUENCE_0046 BCR046 IgH CDR1 Cell_046 Mus_musculus
GYAFSSSW

SEQ ID NO: 47 - protein, 8 aa, Mus musculus
  >SEQUENCE_0047 BCR047 IgH CDR1 Cell_047 Mus_musculus
GYTFTSYW

SEQ ID NO: 48 - protein, 8 aa, Mus musculus
  >SEQUENCE_0048 BCR048 IgH CDR1 Cell_048 Mus_musculus
GYTFTDYY

SEQ ID NO: 49 - protein, 8 aa, Mus musculus
  >SEQUENCE_0049 BCR049 IgH CDR1 Cell_049 Mus_musculus
GYTFTDYY

SEQ ID NO: 50 - protein, 8 aa, Mus musculus
  >SEQUENCE_0050 BCR050 IgH CDR1 Cell_050 Mus_musculus
GFNIKDDY

SEQ ID NO: 51 - protein, 9 aa, Mus musculus
  >SEQUENCE_0051 BCR051 IgH CDR1 Cell_051 Mus_musculus
GFPITSGYY

SEQ ID NO: 52 - protein, 8 aa, Mus musculus
  >SEQUENCE_0052 BCR052 IgH CDR1 Cell_052 Mus_musculus
GYTFTSYW

SEQ ID NO: 53 - protein, 8 aa, Mus musculus
  >SEQUENCE_0053 BCR053 IgH CDR1 Cell_053 Mus_musculus
GYTFTSYG

SEQ ID NO: 54 - protein, 8 aa, Mus musculus
  >SEQUENCE_0054 BCR054 IgH CDR1 Cell_054 Mus_musculus
GYTFTDYY

SEQ ID NO: 55 - protein, 8 aa, Mus musculus
  >SEQUENCE_0055 BCR055 IgH CDR1 Cell_055 Mus_musculus
GYTFTSYW

SEQ ID NO: 56 - protein, 8 aa, Mus musculus
  >SEQUENCE_0056 BCR056 IgH CDR1 Cell_056 Mus_musculus
GYTFTSYG

SEQ ID NO: 57 - protein, 8 aa, Mus musculus
  >SEQUENCE_0057 BCR057 IgH CDR1 Cell_057 Mus_musculus
GCTFSSYA

SEQ ID NO: 58 - protein, 8 aa, Mus musculus
  >SEQUENCE_0058 BCR058 IgH CDR1 Cell_058 Mus_musculus
GFNIKDYY

SEQ ID NO: 59 - protein, 8 aa, Mus musculus
  >SEQUENCE_0059 BCR001 IgH CDR2 Cell_001 Mus_musculus
IYPVIGDT

SEQ ID NO: 60 - protein, 8 aa, Mus musculus
  >SEQUENCE_0060 BCR002 IgH CDR2 Cell_002 Mus_musculus
IYPGDGDT

SEQ ID NO: 61 - protein, 8 aa, Mus musculus
  >SEQUENCE_0061 BCR003 IgH CDR2 Cell_003 Mus_musculus
IYPGDGDT

SEQ ID NO: 62 - protein, 8 aa, Mus musculus
  >SEQUENCE_0062 BCR004 IgH CDR2 Cell_004 Mus_musculus
INPSNGGT

SEQ ID NO: 63 - protein, 8 aa, Mus musculus
  >SEQUENCE_0063 BCR005 IgH CDR2 Cell_005 Mus_musculus
INPTTGGT

SEQ ID NO: 64 - protein, 8 aa, Mus musculus
  >SEQUENCE_0064 BCR006 IgH CDR2 Cell_006 Mus_musculus
INPTTGGT

SEQ ID NO: 65 - protein, 8 aa, Mus musculus
  >SEQUENCE_0065 BCR007 IgH CDR2 Cell_007 Mus_musculus
INPNKGDT

SEQ ID NO: 66 - protein, 8 aa, Mus musculus
  >SEQUENCE_0066 BCR008 IgH CDR2 Cell_008 Mus_musculus
INPSNGGT

SEQ ID NO: 67 - protein, 7 aa, Mus musculus
  >SEQUENCE_0067 BCR009 IgH CDR2 Cell_009 Mus_musculus
IWGDGRT

SEQ ID NO: 68 - protein, 8 aa, Mus musculus
  >SEQUENCE_0068 BCR010 IgH CDR2 Cell_010 Mus_musculus
INPNNGGT

SEQ ID NO: 69 - protein, 8 aa, Mus musculus
  >SEQUENCE_0069 BCR011 IgH CDR2 Cell_011 Mus_musculus
INPNNGGT

SEQ ID NO: 70 - protein, 8 aa, Mus musculus
  >SEQUENCE_0070 BCR012 IgH CDR2 Cell_012 Mus_musculus
INPSNGAP

SEQ ID NO: 71 - protein, 8 aa, Mus musculus
  >SEQUENCE_0071 BCR013 IgH CDR2 Cell_013 Mus_musculus
INPSNGGT

SEQ ID NO: 72 - protein, 8 aa, Mus musculus
  >SEQUENCE_0072 BCR014 IgH CDR2 Cell_014 Mus_musculus
INPYNVST

SEQ ID NO: 73 - protein, 8 aa, Mus musculus
  >SEQUENCE_0073 BCR015 IgH CDR2 Cell_015 Mus_musculus
IYPGDGDT

SEQ ID NO: 74 - protein, 8 aa, Mus musculus
  >SEQUENCE_0074 BCR016 IgH CDR2 Cell_016 Mus_musculus
INPNTGRI

SEQ ID NO: 75 - protein, 8 aa, Mus musculus
  >SEQUENCE_0075 BCR017 IgH CDR2 Cell_017 Mus musculus
INPSTGGT

SEQ ID NO: 76 - protein, 8 aa, Mus musculus
  >SEQUENCE_0076 BCR018 IgH CDR2 Cell_018 Mus_musculus
INPYNGDT

SEQ ID NO: 77 - protein, 8 aa, Mus musculus
  >SEQUENCE_0077 BCR019 IgH CDR2 Cell_019 Mus_musculus
INPNNGGI

SEQ ID NO: 78 - protein, 8 aa, Mus musculus
  >SEQUENCE_0078 BCR020 IgH CDR2 Cell_020 Mus_musculus
IYPGDGDT

SEQ ID NO: 79 - protein, 8 aa, Mus musculus
  >SEQUENCE_0079 BCR021 IgH CDR2 Cell_021 Mus_musculus
INPSTGDI

SEQ ID NO: 80 - protein, 8 aa, Mus musculus
  >SEQUENCE_0080 BCR022 IgH CDR2 Cell_022 Mus_musculus
INPSSDYS

SEQ ID NO: 81 - protein, 8 aa, Mus musculus
  >SEQUENCE_0081 BCR023 IgH CDR2 Cell_023 Mus_musculus
INPNNGVP

SEQ ID NO: 82 - protein, 8 aa, Mus musculus
  >SEQUENCE_0082 BCR024 IgH CDR2 Cell_024 Mus_musculus
INPTTGNT

SEQ ID NO: 83 - protein, 7 aa, Mus musculus
  >SEQUENCE_0083 BCR025 IgH CDR2 Cell_025 Mus_musculus
IWSGGST

SEQ ID NO: 84 - protein, 8 aa, Mus musculus
  >SEQUENCE_0084 BCR026 IgH CDR2 Cell_026 Mus_musculus
IDPNRGGT

SEQ ID NO: 85 - protein, 8 aa, Mus musculus
  >SEQUENCE_0085 BCR027 IgH CDR2 Cell_027 Mus_musculus
IYPGSGNT

SEQ ID NO: 86 - protein, 8 aa, Mus musculus
  >SEQUENCE_0086 BCR028 IgH CDR2 Cell_028 Mus_musculus
INPTTGNT

SEQ ID NO: 87 - protein, 8 aa, Mus musculus
  >SEQUENCE_0087 BCR029 IgH CDR2 Cell_029 Mus_musculus
INPSNGGT

SEQ ID NO: 88 - protein, 8 aa, Mus musculus
  >SEQUENCE_0088 BCR030 IgH CDR2 Cell_030 Mus_musculus
INPSSDYS

SEQ ID NO: 89 - protein, 8 aa, Mus musculus
  >SEQUENCE_0089 BCR031 IgH CDR2 Cell_031 Mus_musculus
IFPGSGST

SEQ ID NO: 90 - protein, 8 aa, Mus musculus
  >SEQUENCE_0090 BCR032 IgH CDR2 Cell_032 Mus_musculus
INPSNGGT

SEQ ID NO: 91 - protein, 8 aa, Mus musculus
  >SEQUENCE_0091 BCR033 IgH CDR2 Cell_033 Mus_musculus
IYPGSSNT

SEQ ID NO: 92 - protein, 8 aa, Mus musculus
  >SEQUENCE_0092 BCR034 IgH CDR2 Cell_034 Mus_musculus
ISSGSSTI

SEQ ID NO: 93 - protein, 8 aa, Mus musculus
  >SEQUENCE_0093 BCR035 IgH CDR2 Cell_035 Mus_musculus
INPNYGTT

SEQ ID NO: 94 - protein, 8 aa, Mus musculus
  >SEQUENCE_0094 BCR036 IgH CDR2 Cell_036 Mus_musculus
INPHKDGA

SEQ ID NO: 95 - protein, 8 aa, Mus musculus
  >SEQUENCE_0095 BCR037 IgH CDR2 Cell_037 Mus_musculus
IYPGDDNT

SEQ ID NO: 96 - protein, 8 aa, Mus musculus
  >SEQUENCE_0096 BCR038 IgH CDR2 Cell_038 Mus_musculus
IDPENGDT

SEQ ID NO: 97 - protein, 8 aa, Mus musculus
  >SEQUENCE_0097 BCR039 IgH CDR2 Cell_039 Mus_musculus
IYPGSGNT

SEQ ID NO: 98 - protein, 8 aa, Mus musculus
  >SEQUENCE_0098 BCR040 IgH CDR2 Cell_040 Mus_musculus
IDPNSGGT

SEQ ID NO: 99 - protein, 8 aa, Mus musculus
  >SEQUENCE_0099 BCR041 IgH CDR2 Cell_041 Mus_musculus
INPSNGGT

SEQ ID NO: 100 - protein, 8 aa, Mus musculus
  >SEQUENCE_0100 BCR042 IgH CDR2 Cell_042 Mus_musculus
IGPGSGST

SEQ ID NO: 101 - protein, 8 aa, Mus musculus
  >SEQUENCE_0101 BCR043 IgH CDR2 Cell_043 Mus_musculus
INPNNGNA

SEQ ID NO: 102 - protein, 8 aa, Mus musculus
  >SEQUENCE_0102 BCR044 IgH CDR2 Cell_044 Mus_musculus
IDPSDSYT

SEQ ID NO: 103 - protein, 8 aa, Mus musculus
  >SEQUENCE_0103 BCR045 IgH CDR2 Cell_045 Mus_musculus
INPNNGDT

SEQ ID NO: 104 - protein, 8 aa, Mus musculus
  >SEQUENCE_0104 BCR046 IgH CDR2 Cell_046 Mus_musculus
IYPGNGET

SEQ ID NO: 105 - protein, 8 aa, Mus musculus
  >SEQUENCE_0105 BCR047 IgH CDR2 Cell_047 Mus_musculus
INPNNGGT

SEQ ID NO: 106 - protein, 8 aa, Mus musculus
  >SEQUENCE_0106 BCR048 IgH CDR2 Cell_048 Mus_musculus
INPKNGGT

SEQ ID NO: 107 - protein, 8 aa, Mus musculus
  >SEQUENCE_0107 BCR049 IgH CDR2 Cell_049 Mus_musculus
INPKNGGT

SEQ ID NO: 108 - protein, 8 aa, Mus musculus
  >SEQUENCE_0108 BCR050 IgH CDR2 Cell_050 Mus_musculus
IDPENGDT

SEQ ID NO: 109 - protein, 7 aa, Mus musculus
  >SEQUENCE_0109 BCR051 IgH CDR2 Cell_051 Mus_musculus
ITHSGET

SEQ ID NO: 110 - protein, 8 aa, Mus musculus
  >SEQUENCE_0110 BCR052 IgH CDR2 Cell_052 Mus_musculus
IDPSDSYT

SEQ ID NO: 111 - protein, 8 aa, Mus musculus
  >SEQUENCE_0111 BCR053 IgH CDR2 Cell_053 Mus_musculus
IYPRSGNT

SEQ ID NO: 112 - protein, 8 aa, Mus musculus
  >SEQUENCE_0112 BCR054 IgH CDR2 Cell_054 Mus_musculus
INPKNGGT

SEQ ID NO: 113 - protein, 8 aa, Mus musculus
  >SEQUENCE_0113 BCR055 IgH CDR2 Cell_055 Mus_musculus
INPNNGNA

SEQ ID NO: 114 - protein, 8 aa, Mus musculus
  >SEQUENCE_0114 BCR056 IgH CDR2 Cell_056 Mus_musculus
IYPRSGNT

SEQ ID NO: 115 - protein, 8 aa, Mus musculus
  >SEQUENCE_0115 BCR057 IgH CDR2 Cell_057 Mus_musculus
ISDGGSYT

SEQ ID NO: 116 - protein, 8 aa, Mus musculus
  >SEQUENCE_0116 BCR058 IgH CDR2 Cell_058 Mus_musculus
IDPEDGET

SEQ ID NO: 117 - protein, 11 aa, Mus musculus
  >SEQUENCE_0117 BCR001 IgH CDR3 Cell_001 Mus_musculus
ASFFYYYIGNY

SEQ ID NO: 118 - protein, 11 aa, Mus musculus
  >SEQUENCE_0118 BCR002 IgH CDR3 Cell_002 Mus_musculus
ARFFYYYGGDY

SEQ ID NO: 119 - protein, 11 aa, Mus musculus
  >SEQUENCE_0119 BCR003 IgH CDR3 Cell_003 Mus_musculus
ARFFYYYGGDY

SEQ ID NO: 120 - protein, 9 aa, Mus musculus
  >SEQUENCE_0120 BCR004 IgH CDR3 Cell_004 Mus_musculus
ARHYLFMDY

SEQ ID NO: 121 - protein, 11 aa, Mus musculus
  >SEQUENCE_0121 BCR005 IgH CDR3 Cell_005 Mus_musculus
ARRELTGSWDY

SEQ ID NO: 122 - protein, 11 aa, Mus musculus
  >SEQUENCE_0122 BCR006 IgH CDR3 Cell_006 Mus_musculus
ARRELTGSWDY

SEQ ID NO: 123 - protein, 8 aa, Mus musculus
  >SEQUENCE_0123 BCR007 IgH CDR3 Cell_007 Mus_musculus
ASRVTRGQ

SEQ ID NO: 124 - protein, 10 aa, Mus musculus
  >SEQUENCE_0124 BCR008 IgH CDR3 Cell_008 Mus_musculus
APITAGYFDV

SEQ ID NO: 125 - protein, 6 aa, Mus musculus
  >SEQUENCE_0125 BCR009 IgH CDR3 Cell_009 Mus_musculus
AGAFAY

SEQ ID NO: 126 - protein, 10 aa, Mus musculus
  >SEQUENCE_0126 BCR010 IgH CDR3 Cell_010 Mus_musculus
ARRLVYYFDY

SEQ ID NO: 127 - protein, 15 aa, Mus musculus
  >SEQUENCE_0127 BCR011 IgH CDR3 Cell_011 Mus_musculus
ARWGDDGYYDWYFDV

SEQ ID NO: 128 - protein, 16 aa, Mus musculus
  >SEQUENCE_0128 BCR012 IgH CDR3 Cell_012 Mus_musculus
ARYGDYYGSSPYAMDY

SEQ ID NO: 129 - protein, 11 aa, Mus musculus
  >SEQUENCE_0129 BCR013 IgH CDR3 Cell_013 Mus_musculus
ARQDYYGCNYY

SEQ ID NO: 130 - protein, 10 aa, Mus musculus
  >SEQUENCE_0130 BCR014 IgH CDR3 Cell_014 Mus_musculus
ARRLVYYFDC

SEQ ID NO: 131 - protein, 14 aa, Mus musculus
  >SEQUENCE_0131 BCR015 IgH CDR3 Cell_015 Mus_musculus
ARRANWDEYWYFDV

SEQ ID NO: 132 - protein, 11 aa, Mus musculus
  >SEQUENCE_0132 BCR016 IgH CDR3 Cell_016 Mus_musculus
ARRELTGSWDY

SEQ ID NO: 133 - protein, 11 aa, Mus musculus
  >SEQUENCE_0133 BCR017 IgH CDR3 Cell_017 Mus_musculus
ARRELTGSWDY

SEQ ID NO: 134 - protein, 13 aa, Mus musculus
  >SEQUENCE_0134 BCR018 IgH CDR3 Cell_018 Mus_musculus
ARGRGGSSYGFAY

SEQ ID NO: 135 - protein, 10 aa, Mus musculus
  >SEQUENCE_0135 BCR019 IgH CDR3 Cell_019 Mus_musculus
VRLGRGYFDV

SEQ ID NO: 136 - protein, 11 aa, Mus musculus
  >SEQUENCE_0136 BCR020 IgH CDR3 Cell_020 Mus_musculus
ARFYYGRGFDY

SEQ ID NO: 137 - protein, 11 aa, Mus musculus
  >SEQUENCE_0137 BCR021 IgH CDR3 Cell_021 Mus_musculus
ARRELTGSWDF

SEQ ID NO: 138 - protein, 12 aa, Mus musculus
  >SEQUENCE_0138 BCR022 IgH CDR3 Cell_022 Mus_musculus
ARLPLLSYPMDY

SEQ ID NO: 139 - protein, 10 aa, Mus musculus
  >SEQUENCE_0139 BCR023 IgH CDR3 Cell_023 Mus_musculus
TRSETGHFDY

SEQ ID NO: 140 - protein, 11 aa, Mus musculus
  >SEQUENCE_0140 BCR024 IgH CDR3 Cell_024 Mus_musculus
ARRELTGSWDY

SEQ ID NO: 141 - protein, 12 aa, Mus musculus
  >SEQUENCE_0141 BCR025 IgH CDR3 Cell_025 Mus_musculus
ASDYYGSRAMDY

SEQ ID NO: 142 - protein, 10 aa, Mus musculus
  >SEQUENCE_0142 BCR026 IgH CDR3 Cell_026 Mus_musculus
SREEMGAMDY

SEQ ID NO: 143 - protein, 11 aa, Mus musculus
  >SEQUENCE_0143 BCR027 IgH CDR3 Cell_027 Mus_musculus
ARGSSSFGFDY

SEQ ID NO: 144 - protein, 11 aa, Mus musculus
  >SEQUENCE_0144 BCR028 IgH CDR3 Cell_028 Mus_musculus
ARRELTGSWDY

SEQ ID NO: 145 - protein, 11 aa, Mus musculus
  >SEQUENCE_0145 BCR029 IgH CDR3 Cell_029 Mus_musculus
ARQDYYGSSYY

SEQ ID NO: 146 - protein, 12 aa, Mus musculus
  >SEQUENCE_0146 BCR030 IgH CDR3 Cell_030 Mus_musculus
ARLPLLSYAMDY

SEQ ID NO: 147 - protein, 14 aa, Mus musculus
  >SEQUENCE_0147 BCR031 IgH CDR3 Cell_031 Mus_musculus
ARGDYYGSSFHFDY

SEQ ID NO: 148 - protein, 10 aa, Mus musculus
  >SEQUENCE_0148 BCR032 IgH CDR3 Cell_032 Mus_musculus
ARSPYSDSYY

SEQ ID NO: 149 - protein, 11 aa, Mus musculus
  >SEQUENCE_0149 BCR033 IgH CDR3 Cell_033 Mus_musculus
ARELTGAWFAY

SEQ ID NO: 150 - protein, 15 aa, Mus musculus
  >SEQUENCE_0150 BCR034 IgH CDR3 Cell_034 Mus_musculus
ARPPPTEAAQAPFAY

SEQ ID NO: 151 - protein, 13 aa, Mus musculus
  >SEQUENCE_0151 BCR035 IgH CDR3 Cell_035 Mus_musculus
ARDYGSSYVWFDY

SEQ ID NO: 152 - protein, 12 aa, Mus musculus
  >SEQUENCE_0152 BCR036 IgH CDR3 Cell_036 Mus_musculus
ARPSYQGGPMDY

SEQ ID NO: 153 - protein, 13 aa, Mus musculus
  >SEQUENCE_0153 BCR037 IgH CDR3 Cell_037 Mus_musculus
AISRGYYGSPFDY

SEQ ID NO: 154 - protein, 13 aa, Mus musculus
  >SEQUENCE_0154 BCR038 IgH CDR3 Cell_038 Mus_musculus
TFYYYGSSYAMDY

SEQ ID NO: 155 - protein, 13 aa, Mus musculus
  >SEQUENCE_0155 BCR039 IgH CDR3 Cell_039 Mus_musculus
ARLHYYGSNAMDH

SEQ ID NO: 156 - protein, 11 aa, Mus musculus
  >SEQUENCE_0156 BCR040 IgH CDR3 Cell_040 Mus_musculus
ARYYYGSYFDY

SEQ ID NO: 157 - protein, 8 aa, Mus musculus
  >SEQUENCE_0157 BCR041 IgH CDR3 Cell_041 Mus_musculus
ASGGLFDY

SEQ ID NO: 158 - protein, 11 aa, Mus musculus
  >SEQUENCE_0158 BCR042 IgH CDR3 Cell_042 Mus_musculus
ARENYYGSLAY

SEQ ID NO: 159 - protein, 10 aa, Mus musculus
  >SEQUENCE_0159 BCR043 IgH CDR3 Cell_043 Mus_musculus
AVYGSGLFAD

SEQ ID NO: 160 - protein, 13 aa, Mus musculus
  >SEQUENCE_0160 BCR044 IgH CDR3 Cell_044 Mus_musculus
ARSYYGSSYGFAY

SEQ ID NO: 161 - protein, 13 aa, Mus musculus
  >SEQUENCE_0161 BCR045 IgH CDR3 Cell_045 Mus_musculus
ARGYYGYDRWFAY

SEQ ID NO: 162 - protein, 16 aa, Mus musculus
  >SEQUENCE_0162 BCR046 IgH CDR3 Cell_046 Mus_musculus
ARFSLWLRRGDWYFDV

SEQ ID NO: 163 - protein, 11 aa, Mus musculus
  >SEQUENCE_0163 BCR047 IgH CDR3 Cell_047 Mus_musculus
ARPNYGSSLDY

SEQ ID NO: 164 - protein, 15 aa, Mus musculus
  >SEQUENCE_0164 BCR048 IgH CDR3 Cell_048 Mus_musculus
ARGGVYYGSNSWFAY

SEQ ID NO: 165 - protein, 15 aa, Mus musculus
  >SEQUENCE_0165 BCR049 IgH CDR3 Cell_049 Mus_musculus
ARGGVYYGSNSWFAY

SEQ ID NO: 166 - protein, 13 aa, Mus musculus
  >SEQUENCE_0166 BCR050 IgH CDR3 Cell_050 Mus_musculus
TFYYYGSSYAMDY

SEQ ID NO: 167 - protein, 12 aa, Mus musculus
  >SEQUENCE_0167 BCR051 IgH CDR3 Cell_051 Mus_musculus
AGDSSGYWYFDV

SEQ ID NO: 168 - protein, 16 aa, Mus musculus
  >SEQUENCE_0168 BCR052 IgH CDR3 Cell_052 Mus_musculus
ARWRYYYGSSEVYFDY

SEQ ID NO: 169 - protein, 16 aa, Mus musculus
  >SEQUENCE_0169 BCR053 IgH CDR3 Cell_053 Mus_musculus
ARSDFEGQLRPYYFDY

SEQ ID NO: 170 - protein, 15 aa, Mus musculus
  >SEQUENCE_0170 BCR054 IgH CDR3 Cell_054 Mus_musculus
ARGGVYYGSNSWFAY

SEQ ID NO: 171 - protein, 11 aa, Mus musculus
  >SEQUENCE_0171 BCR055 IgH CDR3 Cell_055 Mus_musculus
ARPNYGSSLDY

SEQ ID NO: 172 - protein, 15 aa, Mus musculus
  >SEQUENCE_0172 BCR056 IgH CDR3 Cell_056 Mus_musculus
ARGGPDYDGYWYFDV

SEQ ID NO: 173 - protein, 11 aa, Mus musculus
  >SEQUENCE_0173 BCR057 IgH CDR3 Cell_057 Mus_musculus
ARASDSSFFDY

SEQ ID NO: 174 - protein, 6 aa, Mus musculus
  >SEQUENCE_0174 BCR058 IgH CDR3 Cell_058 Mus_musculus
ASNFDY

SEQ ID NO: 175 - protein, 119 aa, Mus musculus
  >SEQUENCE_0175 BCR001 IgH Full length Cell_001 Mus_musculus
EVQLQESGPELVKPGASVKISCKASGYAFSSSWMNWVKQRPGKGLEWIGRIYPVIGDTNYHGKLKGKATLTAYKSSSPAYMQLCNLTSEDSAAYFCASFFYYYIGNYWAQGTTLPLSSS

SEQ ID NO: 176 - protein, 118 aa, Mus musculus
  >SEQUENCE_0176 BCR002 IgH Full length Cell_002 Mus_musculus
EVQLQESGPELVKPGASVKISCKASGYAFSSSWMNWVKQRPGKGLEWIGRIYPGDGDTNYNGKFKGKATLTADKSSSTAYMQLSSLTSEDSAVYFCARFFYYYGGDYWGQGTTLTVSS

SEQ ID NO: 177 - protein, 118 aa, Mus musculus
  >SEQUENCE_0177 BCR003 IgH Full length Cell_003 Mus_musculus
QVQLQQSGPLLVKPGASVKISCKASGYAFSSSWMNWVKQRPGKGLEWIGRIYPGDGDTNYNGKFKGKATLTADKSSSTAYMQLSSLTSEDSAVYFCARFFYYYGGDYWGQGTTLTVSS

SEQ ID NO: 178 - protein, 116 aa, Mus musculus
  >SEQUENCE_0178 BCR004 IgH Full length Cell_004 Mus_musculus
QVLLQESGTELVKPGASVKLSCKASGYTFTSYWMHWVKQRPGQGLEWIGNINPSNGGTNYNEKFKSKATLTVDKSSSTAYLQLSSLTSEDSAVYYCARHYLFMDYWGQGTSVTVSS

SEQ ID NO: 179 - protein, 118 aa, Mus musculus
  >SEQUENCE_0179 BCR005 IgH Full length Cell_005 Mus_musculus
EVQLQESGPELVKPGASVKISCKASGYSFTGYYMNWVKQSPEKSLEWIGEINPTTGGTSYNQNFKAKATLTVDKSSSTAFMQLKSLTSEDSAVYYCARRELTGSWDYWGQGTSVTVSS

SEQ ID NO: 180 - protein, 118 aa, Mus musculus; identical to SEQ ID NO: 179
  >SEQUENCE_0180 BCR006 IgH Full length Cell_006 Mus_musculus

SEQ ID NO: 181 - protein, 115 aa, Mus musculus
  >SEQUENCE_0181 BCR007 IgH Full length Cell_007 Mus_musculus
EVQLQESGTELVKPGASVKLSCKDSGYTFTSSWMHRVKPRPGQGLDWIGNINPNKGDTNYNERFRTKATLTVDKSSSTAYMQLSSLTSEDSAVYSCASRVTRGQRGQGTLVPVSE

SEQ ID NO: 182 - protein, 117 aa, Mus musculus
  >SEQUENCE_0182 BCR008 IgH Full length Cell_008 Mus_musculus
EVQLQESGTELVKPGASVKLSCKASGYTFTSYWMHWVKQRPGQGLEWIGNINPSNGGTNYNEKFKSKATLTVDKSSSTAYMQLSSLTSEDSAVYYCAPITAGYFDVWGTGTTVTVSS

SEQ ID NO: 183 - protein, 112 aa, Mus musculus
  >SEQUENCE_0183 BCR009 IgH Full length Cell_009 Mus_musculus
EVQLQESGPGLVAPSQSLSITCTVSGFSLTNYGVTWVRQPPGKGLEWLGGIWGDGRTNYHSALISRLSISKDNSKSQVFLKLNSLQTDDTATYYCAGAFAYWGQGTLVTVSA

SEQ ID NO: 184 - protein, 117 aa, Mus musculus
  >SEQUENCE_0184 BCR010 IgH Full length Cell_010 Mus_musculus
EVQLQESGPELVKPGASVKISCKASGFTFTDYYMNWVKQSHGKSLEWIGDINPNNGGTNYHQKFKDKATLTVDKSSSTAYMELRSLTSEDSAVYYCARRLVYYFDYWGQGTTLTVSS

SEQ ID NO: 185 - protein, 122 aa, Mus musculus
  >SEQUENCE_0185 BCR011 IgH Full length Cell_011 Mus_musculus
EVQLQESGPELVKPGASVKISCKASGYTFTDYYMNWVKQSHGKSLEWIGDINPNNGGTSYNQKFKGKATLTVDKSSSTAYMEFRSLTSEDSAVYYCARWGDDGYYDWYFDVWGTGTTVTVSS

SEQ ID NO: 186 - protein, 123 aa, Mus musculus
  >SEQUENCE_0186 BCR012 IgH Full length Cell_012 Mus_musculus
EVQLQESGTELVKPGASVKLSCKASGYTFTSYWMHWVKQRPGQGLEWIGNINPSNGAPNYNEEFKSKATLTVDKSSSTAYMQLSSLTSEDSAVYYCARYGDYYGSSPYAMDYWGQGTSVTVSS

SEQ ID NO: 187 - protein, 118 aa, Mus musculus
  >SEQUENCE_0187 BCR013 IgH Full length Cell_013 Mus_musculus
EVQLQESGTELVKPGASVKLSCKASGYTFTSYWMHWVKQRPGQGLEWIGNINPSNGGTNYNEKFKSKATLTVDKSSSTAYMQLSSLTSEDSAVYYCARQDYYGCNYYWGQGTTLTVSS

SEQ ID NO: 188 - protein, 117 aa, Mus musculus
  >SEQUENCE_0188 BCR014 IgH Full length Cell_014 Mus_musculus
EVQLQESGPELVKPGASVKISCKASGFTFTDYYMNWVKQSHGKSLEWIGDINPYNVSTNYHQKFKDKATLTVDKSSSTAYMELRSLTSEDSAVYYCARRLVYYFDCWGQGTTLTVSS

SEQ ID NO: 189 - protein, 121 aa, Mus musculus
  >SEQUENCE_0189 BCR015 IgH Full length Cell_015 Mus_musculus
EVQLQESGPELVKPGASVKISCKASGYAFSSSWMNWVKQRPGKGLEWIGRIYPGDGDTNYNGKFKGKATLTADKSSSTAYMQLSSLTSEDSAVYFCARRANWDEYWYFDVWGTGTTVTVSS

SEQ ID NO: 190 - protein, 118 aa, Mus musculus
  >SEQUENCE_0190 BCR016 IgH Full length Cell_016 Mus_musculus
EVQLQESGPELVKPGASVKISCKASGYSFTAYYMNWVKQSPEKSLEWIGEINPNTGRITDNQNFKAKATLTVDKSSSTAFMQLKSLTSEDSAVYYCARRELTGSWDYWGQGTSVTVSS

SEQ ID NO: 191 - protein, 118 aa, Mus musculus
  >SEQUENCE_0191 BCR017 IgH Full length Cell_017 Mus_musculus
EVQLQESGPELVKPGASVKISCKASGYSFTGYYMNWVKQSPEKSLEWIGEINPSTGGTTYNQNFKAKATLTVDKSSSTAFMQLKSLTSEDSAVYYCARRELTGSWDYWGQGTSVTVSS

SEQ ID NO: 192 - protein, 120 aa, Mus musculus
  >SEQUENCE_0192 BCR018 IgH Full length Cell_018 Mus_musculus
EVQLQESGPELVKPGDSVKISCKASGYSFTGYFMNWVMQSHGKSLEWIGRINPYNGDTFYNQKFKGKATLTVDKSSSTAHMELRSLTSEDSAVYYCARGRGGSSYGFAYWGQGTLVTVSA

SEQ ID NO: 193 - protein, 117 aa, Mus musculus
  >SEQUENCE_0193 BCR019 IgH Full length Cell_019 Mus_musculus
EVQLQESGPELVKPGASVKISCKASGYTFTDYYMNWVKQSHGKSLEWIGDINPNNGGINYNQTFKGKATLTVDKSSITAYMELRSLTSEDSAVYYCVRLGRGYFDVWGTGTTVTVSS

SEQ ID NO: 194 - protein, 118 aa, Mus musculus
  >SEQUENCE_0194 BCR020 IgH Full length Cell_020 Mus_musculus
EVQLQESGAEVVKPGASVKISCKDFGYAFSNYWMNWVKQRPGKGLEWIGQIYPGDGDTKYNGKFKGKATLTADKSSSTAYMQLSSLTSEDSAVYFCARFYYGRGFDYWGQGTTLTVSS

SEQ ID NO: 195 - protein, 118 aa, Mus musculus
  >SEQUENCE_0195 BCR021 IgH Full length Cell_021 Mus_musculus
EVQLQESGPELVKPGTSVKISCKASGYSFSGYYMNWVKQSPEKSLEWIGQINPSTGDITYNQNFKAKATLTVDKSSSTAFMQLRSLTSEDSAVYYCARRELTGSWDFWGQGTSVTVSS

SEQ ID NO: 196 - protein, 119 aa, Mus musculus
  >SEQUENCE_0196 BCR022 IgH Full length Cell_022 Mus_musculus
EVQLQESGAELARPGASVKMSCKASGYTFTNYTMHWIKQRPGQGLEWIGYINPSSDYSRYSQKFKDKATLTADKSSSTAYMQLNSLTSEDSAVYYCARLPLLSYPMDYWGQGTSVTVSS

SEQ ID NO: 197 - protein, 117 aa, Mus musculus
  >SEQUENCE_0197 BCR023 IgH Full length Cell_023 Mus_musculus
EVQLQESGTELVKPGASVKLSCKASGYTFTSYWMHWVKQRPGQGLEWIGNINPNNGVPSYNEKFKSKATLTVDKSSSTAYMQLSSLTSEDSAVYYCTRSETGHFDYWGQGTTLTVSS

SEQ ID NO: 198 - protein, 118 aa, Mus musculus
  >SEQUENCE_0198 BCR024 IgH Full length Cell_024 Mus_musculus
EVQLQESGPELVKPGASVKISCKASGYSFTGYYMNWVKQSPEKSLEWIGEINPTTGNTTYNQHFKAKATLTVDKSSSTAYMQFKSLTSEDSAVYYCARRELTGSWDYWGQGTSVTVSS

SEQ ID NO: 199 - protein, 118 aa, Mus musculus
  >SEQUENCE_0199 BCR025 IgH Full length Cell_025 Mus_musculus
EVQLQESGPGLVQPSQSLSITCTVSGFSLTSYGVHWVRQSPGKGLEWLGVIWSGGSTDYNAAFISRLSISKDNSKSQVFFKMNSLQADDTAIYYCASDYYGSRAMDYWGQGTSVTVSS

SEQ ID NO: 200 - protein, 117 aa, Mus musculus
  >SEQUENCE_0200 BCR026 IgH Full length Cell_026 Mus_musculus
EVQLQESGAVLVKPGTSVKLSCKASGSTFTSYWMHWVQQRPGRGLEWIGRIDPNRGGTENNGKFKSKASLTVDKPSSTVYMQLSSLTSEDSAVYYCSREEMGAMDYWGQGTSVTVSS

SEQ ID NO: 201 - protein, 118 aa, Mus musculus
  >SEQUENCE_0201 BCR027 IgH Full length Cell_027 Mus_musculus
EVQLQESGAELVRPGASVKLSCKASGYTFTDYYINWVKQRPGQGLEWIARIYPGSGNTYYNEKFKGKATLTAEKSSSTAYMQLSSLTSEDSAVYFCARGSSSFGFDYWGQGTTLTVSS

SEQ ID NO: 202 - protein, 118 aa, Mus musculus
  >SEQUENCE_0202 BCR028 IgH Full length Cell_028 Mus_musculus
EVQLQESGPELVKPGASVKISCKASGYSFTAYYMNWVKQSPEKSLEWIGEINPTTGNTTYNQKFKAKATLTVDKSSRTAYMQFKSLTSEDSAVYYCARRELTGSWDYWGQGTSVTVSS

SEQ ID NO: 203 - protein, 118 aa, Mus musculus
  >SEQUENCE_0203 BCR029 IgH Full length Cell_029 Mus_musculus
EVQLQESGTELVKPGASVKLSYKASGYTFTSYWMHWVKQRPGQGLEWIGNINPSNGGTNYNEKFKSKATLTVDKSSSTAYMQLSSLTFEDSAVYYCARQDYYGSSYYWGQGTTLTVSS

SEQ ID NO: 204 - protein, 119 aa, Mus musculus
  >SEQUENCE_0204 BCR030 IgH Full length Cell_030 Mus_musculus
QVQLQESGAELARPGASVKMSCKASGYTFTNYTMHWIKQRPGQGLEWIGYINPSSDYSKYNQKFKDKATLTADKSSSTAYMQLNSLTSEDSAVYYCARLPLLSYAMDYWGQGTSVTVSS

SEQ ID NO: 205 - protein, 121 aa, Mus musculus
  >SEQUENCE_0205 BCR031 IgH Full length Cell_031 Mus_musculus
QVQLQQSGPEVVKPGASAKFSCKASGYTFTDYYINRVKQRPGQGLEWIGWIFPGSGSTYYNEKFKGKATLTVDKSSSTGYMLLSSLTSEDSAVYFCARGDYYGSSFHFDYWGQGTILTVSS

SEQ ID NO: 206 - protein, 117 aa, Mus musculus
  >SEQUENCE_0206 BCR032 IgH Full length Cell_032 Mus_musculus
QVQLQQPGTVLGTSEASVKLSCKASGYTFTSYWMHWVKQRPGQGLEWIGNINPSNGGTNYNEKFKSKATLTVDKSSTTAYMQLSSLTSEDSAVYYCARSPYSDSYYWGQGTTLTVSS

SEQ ID NO: 207 - protein, 118 aa, Mus musculus
  >SEQUENCE_0207 BCR033 IgH Full length Cell_033 Mus_musculus
EVLLQESGAELVRPGASVKLSCKASGYTFTDYYINWVKQRPGQGLEWIARIYPGSSNTYYNEKFKGKATLTAEKSSSTAYMQLSSLTSEDSAVYFCARELTGAWFAYWGQGTLVTVSV

SEQ ID NO: 208 - protein, 122 aa, Mus musculus
  >SEQUENCE_0208 BCR034 IgH Full length Cell_034 Mus_musculus
EVQLVESGGGLVKPGESPELSCAASGFTFSDYGMHWVRQAPEKGLEWVAYISSGSSTIYYADTVKGRFTISRDNAKNTLFLQMTSLRSEDTAMYYCARPPPTEAAQAPFAYWGQGTLVTVSA

SEQ ID NO: 209 - protein, 120 aa, Mus musculus
  >SEQUENCE_0209 BCR035 IgH Full length Cell_035 Mus_musculus
QFQLQQSGPELVKPGASVKISCKASGYSFIDYNMNWVKQSNGKSLEWIGVINPNYGTTRYNQKFKGKATLTVDQSSSTAYMQLNRLTSEDSAVYYCARDYGSSYVWFDYWGQGTLVTVSA

SEQ ID NO: 210 - protein, 119 aa, Mus musculus
  >SEQUENCE_0210 BCR036 IgH Full length Cell_036 Mus_musculus
EVQLQESGPELVKPGASVKISCKASGYTFTDYYMKWVKQSHGKSLEWIGDINPHKDGATYNQKFKGKATLIVDKSSSTAYMELRSLTSEDSAVYFCARPSYQGGPMDYWGQGTSVTVSS

SEQ ID NO: 211 - protein, 120 aa, Mus musculus
  >SEQUENCE_0211 BCR037 IgH Full length Cell_037 Mus_musculus
EVQLQESGAELVKPGASVKISCKASDYAFSNYWMNWVKQRPGKGLEWIGQIYPGDDNTNYSGKFKGTATLTADKSSSTAYMHLRSLTSEDSAVYFCAISRGYYGSPFDYWGPGTTLTVSS

SEQ ID NO: 212 - protein, 120 aa, Mus musculus
  >SEQUENCE_0212 BCR038 IgH Full length Cell_038 Mus_musculus
EVQLQESGAELVRPGASVKLSCTASGFNIKDDYMHWVKQRPEQGLEWIGWIDPENGDTEYASKFQGKATITADTSSNTAYLQLSSLTSEDTAVYYCTFYYYGSSYAMDYWGQGTSVTVSS

SEQ ID NO: 213 - protein, 120 aa, Mus musculus
  >SEQUENCE_0213 BCR039 IgH Full length Cell_039 Mus_musculus
EVQLQESGPELVKPGASVKISCKASGYTFTDYYINWVKQRPGQGLEWIGWIYPGSGNTKYNAKFKGKATLTVDTSSSTAYMQLSSLTSEDSAVYFCARLHYYGSNAMDHWGQGTSVTVSS

SEQ ID NO: 214 - protein, 118 aa, Mus musculus
  >SEQUENCE_0214 BCR040 IgH Full length Cell_040 Mus_musculus
EVQLQESGAELVKPGASVKLSCKASGYIFTSYWMHWVKQRPGRGLEWIGRIDPNSGGTKYNEKFKSKATLTVDKPSSTAYMQLSSLTSEDSAVYYCARYYYGSYFDYWGQGTTLTVSS

SEQ ID NO: 215 - protein, 115 aa, Mus musculus
  >SEQUENCE_0215 BCR041 IgH Full length Cell_041 Mus_musculus
EVQLQESGTELVKPGASVKLSCRASGHTFTSHWMHWVKQRPGQGLEWIGNINPSNGGTNYNEKFKSKATLTVDKSSRTAYMQVSSLTFEDVAVYYCASGGLFDYWGQGTTVTVSS

SEQ ID NO: 216 - protein, 118 aa, Mus musculus
  >SEQUENCE_0216 BCR042 IgH Full length Cell_042 Mus_musculus
EVQLQESGAELVKPGASVKISCKASGYTFTDYYINWVKQRPGQGLEWIGKIGPGSGSTYYNEKFKGKATLTADKSSSTAYMQLSSLTSEDSAVYFCARENYYGSLAYWGQGTLVTVSA

SEQ ID NO: 217 - protein, 117 aa, Mus musculus
  >SEQUENCE_0217 BCR043 IgH Full length Cell_043 Mus_musculus
QVQLQESGTELVKPGTSAKLSCKASGYTFTSYWMHWVKQRPGQGLEWIGNINPNNGNANYNEKFKNKATLTVDKSSNTAYMQLSSLTSDDSAVYYCAVYGSGLFADWGQGSALTVSS

SEQ ID NO: 218 - protein, 120 aa, Mus musculus
  >SEQUENCE_0218 BCR044 IgH Full length Cell_044 Mus_musculus
QVQLQESGAELVKPGASVKLSCKASGYTFTSYWMQWVKQRPGQGLEWIGEIDPSDSYTNYNQKFKGKATLTVDTSSSTAYMQLSSLTSEDSAVYYCARSYYGSSYGFAYWGQGTLVTVSA

SEQ ID NO: 219 - protein, 120 aa, Mus musculus
  >SEQUENCE_0219 BCR045 IgH Full length Cell_045 Mus_musculus
EVQLQESGPELVKPGASVKISCKASGYTFTDYYMSWVKQSHGKSLAWIGHINPNNGDTDYAQKFKGKATVTIDKSSSTADMELRSLTSEDSAVYYCARGYYGYDRWFAYWGQGTLVTVSA

SEQ ID NO: 220 - protein, 123 aa, Mus musculus
  >SEQUENCE_0220 BCR046 IgH Full length Cell_046 Mus_musculus
EVQLQESGPELVKPGASVKISCKASGYAFSSSWMNWVKQRPGKGLEWIGRIYPGNGETNYNGKFKGKATLTTDKSSTTAYMQLSSLTSEDSAVYFCARFSLWLRRGDWYFDVWGTGTTVTVSS

SEQ ID NO: 221 - protein, 118 aa, Mus musculus
  >SEQUENCE_0221 BCR047 IgH Full length Cell_047 Mus_musculus
EVQLQESGTELVKPGTSAKLSCKASGYTFTSYWMHWVKQRPGQGLEWIGNINPNNGGTNYNEKFKNKATLTVDKSSSTAYMQLSSLTSEDSAVYYCARPNYGSSLDYWGQGSTLTVSS

SEQ ID NO: 222 - protein, 122 aa, Mus musculus
  >SEQUENCE_0222 BCR048 IgH Full length Cell_048 Mus_musculus
EVQLQESGPELVKPGASVKISCKASGYTFTDYYMNWVKQSHGKSLEWIGNINPKNGGTSYNQKFKGKATLTVDRSSSTAYMELRSLTSEDSAVYYCARGGVYYGSNSWFAYWGQGTLVTVSA

SEQ ID NO: 223 - protein, 122 aa, Mus musculus; identical to SEQ ID NO: 222
  >SEQUENCE_0223 BCR049 IgH Full length Cell_049 Mus_musculus

SEQ ID NO: 224 - protein, 120 aa, Mus musculus; identical to SEQ ID NO: 212
  >SEQUENCE_0224 BCR050 IgH Full length Cell_050 Mus_musculus

SEQ ID NO: 225 - protein, 119 aa, Mus musculus
  >SEQUENCE_0225 BCR051 IgH Full length Cell_051 Mus_musculus
EVQLQESGPGLVKPSQSLFLTCSITGFPITSGYYWIWIRQSPGKPLEWMGYITHSGETFYNPSLQSPISITRETSKNQFFLQLNSVTTEDTAMYYCAGDSSGYWYFDVWGTGTTVTVSS

SEQ ID NO: 226 - protein, 123 aa, Mus musculus
  >SEQUENCE_0226 BCR052 IgH Full length Cell_052 Mus_musculus
EVQLQESGAELVKPGASVKLSCKASGYTFTSYWMQWVKQRPGQGLEWIGEIDPSDSYTNYNQKFKGKATLTVDTSSSTAYMQLSSLTSEDSAVYYCARWRYYYGSSEVYFDYWGQGTTLTVSS

SEQ ID NO: 227 - protein, 123 aa, Mus musculus
  >SEQUENCE_0227 BCR053 IgH Full length Cell_053 Mus_musculus
EVQLQESGAELARPGASVKLSCKASGYTFTSYGISWVKQRTGQGLEWIGEIYPRSGNTYYNEKFKGKATLTADKSSSTAYMELRSLTSEDSAVYFCARSDFEGQLRPYYFDYWGQGTTLTVSS

SEQ ID NO: 228 - protein, 122 aa, Mus musculus
  >SEQUENCE_0228 BCR054 IgH Full length Cell_054 Mus_musculus
EVQLQESGPELVKPGASVKISCKASGYTFTDYYMNWVKQSHGKSLEWIGNINPKNGGTSYNQKFKGKATLTVDRSSSTAYMELRSLTSEDSAVYYCARGGVYYGSNSWFAYWGQGTLVTVFA

SEQ ID NO: 229 - protein, 118 aa, Mus musculus
  >SEQUENCE_0229 BCR055 IgH Full length Cell_055 Mus_musculus
EVQLQESGTELVKPGTSAKLSCKASGYTFTSYWMHWVKQRPGQGLEWIGNINPNNGNANYNEKFKNKATLTVDKSSNTAYMQLSSLTSDDSAVYYCARPNYGSSLDYWGQGSALTVSS

SEQ ID NO: 230 - protein, 122 aa, Mus musculus
  >SEQUENCE_0230 BCR056 IgH Full length Cell_056 Mus_musculus
EVQLQESGAELARPGASVKLSCKASGYTFTSYGISWVKQRTGQGLEWIGEIYPRSGNTYYNEKFKGKATLAADKSSSTAYMELRSLTSEDSAVYFCARGGPDYDGYWYFDVWGTGTTVTVSS

SEQ ID NO: 231 - protein, 118 aa, Mus musculus
  >SEQUENCE_0231 BCR057 IgH Full length Cell_057 Mus_musculus
EVQLQESGGDLVKPGGSLKLSCAASGCTFSSYAMSWVRQTPEKRLEWVATISDGGSYTYYPDNVKGRFTISRDNAKNNLYLQMSHLKSEDTAMYYCARASDSSFFDYWGQGTTLTVSS

SEQ ID NO: 232 - protein, 113 aa, Mus musculus
  >SEQUENCE_0232 BCR058 IgH Full length Cell_058 Mus_musculus
EVQLQESGAELVKPGASVKLSCTASGFNIKDYYMHWVKQRTEQGLEWIGRIDPEDGETKYAPKFQGKATITADTSSNTAYLQLSSLTSEDTAVYYCASNFDYWGQGTTLTVSS

SEQ ID NO: 233 - protein, 6 aa, Mus musculus
  >SEQUENCE_0233 BCR001 IgL CDR1 Cell_001 Mus_musculus
QSISDY

SEQ ID NO: 234 - protein, 6 aa, Mus musculus
  >SEQUENCE_0234 BCR002 IgL CDR1 Cell_002 Mus_musculus
TDIDDD

SEQ ID NO: 235 - protein, 10 aa, Mus musculus
  >SEQUENCE_0235 BCR003 IgL CDR1 Cell_003 Mus_musculus
ESVESYGNSF

SEQ ID NO: 236 - protein, 10 aa, Mus musculus
  >SEQUENCE_0236 BCR004 IgL CDR1 Cell_004 Mus_musculus
ESVDSYGNSF

SEQ ID NO: 237 - protein, 10 aa, Mus musculus
  >SEQUENCE_0237 BCR005 IgL CDR1 Cell_005 Mus_musculus
ESVDIYGKSF

SEQ ID NO: 238 - protein, 10 aa, Mus musculus
  >SEQUENCE_0238 BCR006 IgL CDR1 Cell_006 Mus_musculus
ESVDNYGISF

SEQ ID NO: 239 - protein, 10 aa, Mus musculus
  >SEQUENCE_0239 BCR007 IgL CDR1 Cell_007 Mus_musculus
ESVDNFGISF

SEQ ID NO: 240 - protein, 10 aa, Mus musculus
  >SEQUENCE_0240 BCR008 IgL CDR1 Cell_008 Mus_musculus
ESVDNFGISF

SEQ ID NO: 241 - protein, 6 aa, Mus musculus
  >SEQUENCE_0241 BCR009 IgL CDR1 Cell_009 Mus_musculus
QSISDY

SEQ ID NO: 242 - protein, 10 aa, Mus musculus
  >SEQUENCE_0242 BCR010 IgL CDR1 Cell_010 Mus_musculus
ESVDNYGISF

SEQ ID NO: 243 - protein, 10 aa, Mus musculus
  >SEQUENCE_0243 BCR011 IgL CDR1 Cell_011 Mus_musculus
ESVDNYGISF

SEQ ID NO: 244 - protein, 10 aa, Mus musculus
  >SEQUENCE_0244 BCR012 IgL CDR1 Cell_012 Mus_musculus
ESVDNYGISF

SEQ ID NO: 245 - protein, 10 aa, Mus musculus
  >SEQUENCE_0245 BCR013 IgL CDR1 Cell_013 Mus_musculus
ESVDSYGNSF

SEQ ID NO: 246 - protein, 10 aa, Mus musculus
  >SEQUENCE_0246 BCR014 IgL CDR1 Cell_014 Mus_musculus
ESVDNYGISF

SEQ ID NO: 247 - protein, 9 aa, Mus musculus
  >SEQUENCE_0247 BCR015 IgL CDR1 Cell_015 Mus_musculus
TGAVTTSNH

SEQ ID NO: 248 - protein, 10 aa, Mus musculus
  >SEQUENCE_0248 BCR016 IgL CDR1 Cell_016 Mus_musculus
ESVDNYGISF

SEQ ID NO: 249 - protein, 10 aa, Mus musculus
  >SEQUENCE_0249 BCR017 IgL CDR1 Cell_017 Mus_musculus
ESVDNYGISF

SEQ ID NO: 250 - protein, 10 aa, Mus musculus
  >SEQUENCE_0250 BCR018 IgL CDR1 Cell_018 Mus_musculus
ETVDNYGISF

SEQ ID NO: 251 - protein, 10 aa, Mus musculus
  >SEQUENCE_0251 BCR019 IgL CDR1 Cell_019 Mus_musculus
ESVDNFGISF

SEQ ID NO: 252 - protein, 6 aa, Mus musculus
  >SEQUENCE_0252 BCR020 IgL CDR1 Cell_020 Mus_musculus
QDVNKF

SEQ ID NO: 253 - protein, 5 aa, Mus musculus
  >SEQUENCE_0253 BCR021 IgL CDR1 Cell_021 Mus_musculus
SSINY

SEQ ID NO: 254 - protein, 10 aa, Mus musculus
  >SEQUENCE_0254 BCR022 IgL CDR1 Cell_022 Mus_musculus
ESVDSYGKSF

SEQ ID NO: 255 - protein, 10 aa, Mus musculus
  >SEQUENCE_0255 BCR023 IgL CDR1 Cell_023 Mus_musculus
DSVDNYGISF

SEQ ID NO: 256 - protein, 10 aa, Mus musculus
  >SEQUENCE_0256 BCR024 IgL CDR1 Cell_024 Mus_musculus
ESVDRYGKSF

SEQ ID NO: 257 - protein, 9 aa, Mus musculus
  >SEQUENCE_0257 BCR025 IgL CDR1 Cell_025 Mus_musculus
TGAVTTSNY

SEQ ID NO: 258 - protein, 12 aa, Mus musculus
  >SEQUENCE_0258 BCR026 IgL CDR1 Cell_026 Mus_musculus
QSLLNSGNQKNY

SEQ ID NO: 259 - protein, 7 aa, Mus musculus
  >SEQUENCE_0259 BCR027 IgL CDR1 Cell_027 Mus_musculus
SSVSSSY

SEQ ID NO: 260 - protein, 6 aa, Mus musculus
  >SEQUENCE_0260 BCR028 IgL CDR1 Cell_028 Mus_musculus
QSISDY

SEQ ID NO: 261 - protein, 6 aa, Mus musculus
  >SEQUENCE_0261 BCR029 IgL CDR1 Cell_029 Mus_musculus
QSISDY

SEQ ID NO: 262 - protein, 10 aa, Mus musculus
  >SEQUENCE_0262 BCR030 IgL CDR1 Cell_030 Mus_musculus
ESVDNYGISF

SEQ ID NO: 263 - protein, 6 aa, Mus musculus
  >SEQUENCE_0263 BCR031 IgL CDR1 Cell_031 Mus_musculus
QNINVW

SEQ ID NO: 264 - protein, 10 aa, Mus musculus
  >SEQUENCE_0264 BCR032 IgL CDR1 Cell_032 Mus_musculus
ESVDNFGISF

SEQ ID NO: 265 - protein, 5 aa, Mus musculus
  >SEQUENCE_0265 BCR033 IgL CDR1 Cell_033 Mus_musculus
SSVSD

SEQ ID NO: 266 - protein, 10 aa, Mus musculus
  >SEQUENCE_0266 BCR034 IgL CDR1 Cell_034 Mus_musculus
ESVDNFGISF

SEQ ID NO: 267 - protein, 10 aa, Mus musculus
  >SEQUENCE_0267 BCR035 IgL CDR1 Cell_035 Mus_musculus
ESVDNYGISF

SEQ ID NO: 268 - protein, 10 aa, Mus musculus
  >SEQUENCE_0268 BCR036 IgL CDR1 Cell_036 Mus_musculus
ESVDSYGNSF

SEQ ID NO: 269 - protein, 7 aa, Mus musculus
  >SEQUENCE_0269 BCR037 IgL CDR1 Cell_037 Mus_musculus
SSVSSSY

SEQ ID NO: 270 - protein, 5 aa, Mus musculus
  >SEQUENCE_0270 BCR038 IgL CDR1 Cell_038 Mus_musculus
SSVNY

SEQ ID NO: 271 - protein, 10 aa, Mus musculus
  >SEQUENCE_0271 BCR039 IgL CDR1 Cell_039 Mus_musculus
ESVDNYGISI

SEQ ID NO: 272 - protein, 10 aa, Mus musculus
  >SEQUENCE_0272 BCR040 IgL CDR1 Cell_040 Mus_musculus
ESVDNYGISF

SEQ ID NO: 273 - protein, 7 aa, Mus musculus
  >SEQUENCE_0273 BCR041 IgL CDR1 Cell_041 Mus_musculus
SSISSSY

SEQ ID NO: 274 - protein, 10 aa, Mus musculus
  >SEQUENCE_0274 BCR042 IgL CDR1 Cell_042 Mus_musculus
ESVDNYGISF

SEQ ID NO: 275 - protein, 10 aa, Mus musculus
  >SEQUENCE_0275 BCR043 IgL CDR1 Cell_043 Mus_musculus
ESVDDYGISF

SEQ ID NO: 276 - protein, 6 aa, Mus musculus
  >SEQUENCE_0276 BCR044 IgL CDR1 Cell_044 Mus_musculus
QGISNY

SEQ ID NO: 277 - protein, 6 aa, Mus musculus
  >SEQUENCE_0277 BCR045 IgL CDR1 Cell_045 Mus_musculus
QNINVW

SEQ ID NO: 278 - protein, 5 aa, Mus musculus
  >SEQUENCE_0278 BCR046 IgL CDR1 Cell_046 Mus_musculus
SSVSY

SEQ ID NO: 279 - protein, 10 aa, Mus musculus
  >SEQUENCE_0279 BCR047 IgL CDR1 Cell_047 Mus_musculus
ESVDNYGISF

SEQ ID NO: 280 - protein, 6 aa, Mus musculus
  >SEQUENCE_0280 BCR048 IgL CDR1 Cell_048 Mus_musculus
QGISNY

SEQ ID NO: 281 - protein, 6 aa, Mus musculus
  >SEQUENCE_0281 BCR049 IgL CDR1 Cell_049 Mus_musculus
QGISNY

SEQ ID NO: 282 - protein, 7 aa, Mus musculus
  >SEQUENCE_0282 BCR050 IgL CDR1 Cell_050 Mus_musculus
SSISSNY

SEQ ID NO: 283 - protein, 7 aa, Mus musculus
  >SEQUENCE_0283 BCR051 IgL CDR1 Cell_051 Mus_musculus
SSISSNY

SEQ ID NO: 284 - protein, 6 aa, Mus musculus
  >SEQUENCE_0284 BCR052 IgL CDR1 Cell_052 Mus_musculus
QSISDY

SEQ ID NO: 285 - protein, 6 aa, Mus musculus
  >SEQUENCE_0285 BCR053 IgL CDR1 Cell_053 Mus_musculus
QDINNY

SEQ ID NO: 286 - protein, 6 aa, Mus musculus
  >SEQUENCE_0286 BCR054 IgL CDR1 Cell_054 Mus_musculus
QGISNY

SEQ ID NO: 287 - protein, 10 aa, Mus musculus
  >SEQUENCE_0287 BCR055 IgL CDR1 Cell_055 Mus_musculus
ESVDDYGISF

SEQ ID NO: 288 - protein, 6 aa, Mus musculus
  >SEQUENCE_0288 BCR056 IgL CDR1 Cell_056 Mus_musculus
QDINSD

SEQ ID NO: 289 - protein, 6 aa, Mus musculus
  >SEQUENCE_0289 BCR057 IgL CDR1 Cell_057 Mus_musculus
QGISNY

SEQ ID NO: 290 - protein, 6 aa, Mus musculus
  >SEQUENCE_0290 BCR058 IgL CDR1 Cell_058 Mus_musculus
QGISNF

SEQ ID NO: 291 - protein, 9 aa, Mus musculus
  >SEQUENCE_0349 BCR001 IgL CDR3 Cell_001 Mus_musculus
QNGHTFPFT

SEQ ID NO: 292 - protein, 9 aa, Mus musculus
  >SEQUENCE_0350 BCR002 IgL CDR3 Cell_002 Mus_musculus
LQSDNMPYT

SEQ ID NO: 293 - protein, 9 aa, Mus musculus
  >SEQUENCE_0351 BCR003 IgL CDR3 Cell_003 Mus_musculus
QQSNEDPWT

SEQ ID NO: 294 - protein, 9 aa, Mus musculus
  >SEQUENCE_0352 BCR004 IgL CDR3 Cell_004 Mus_musculus
QQNNEDPYT

SEQ ID NO: 295 - protein, 9 aa, Mus musculus
  >SEQUENCE_0353 BCR005 IgL CDR3 Cell_005 Mus_musculus
QQSNEDPYT

SEQ ID NO: 296 - protein, 9 aa, Mus musculus
  >SEQUENCE_0354 BCR006 IgL CDR3 Cell_006 Mus_musculus
QQSKEVPYT

SEQ ID NO: 297 - protein, 9 aa, Mus musculus
  >SEQUENCE_0355 BCR007 IgL CDR3 Cell_007 Mus_musculus
QQSKEVPWT

SEQ ID NO: 298 - protein, 9 aa, Mus musculus
  >SEQUENCE_0356 BCR008 IgL CDR3 Cell_008 Mus_musculus
QQSKEVPLT

SEQ ID NO: 299 - protein, 9 aa, Mus musculus
  >SEQUENCE_0357 BCR009 IgL CDR3 Cell_009 Mus_musculus
QNGHTFPFT

SEQ ID NO: 300 - protein, 9 aa, Mus musculus
  >SEQUENCE_0358 BCR010 IgL CDR3 Cell_010 Mus_musculus
QQSKEVPYT

SEQ ID NO: 301 - protein, 9 aa, Mus musculus
  >SEQUENCE_0359 BCR011 IgL CDR3 Cell_011 Mus_musculus
QQSKEVPRT

SEQ ID NO: 302 - protein, 9 aa, Mus musculus
  >SEQUENCE_0360 BCR012 IgL CDR3 Cell_012 Mus_musculus
QQSKEVPWT

SEQ ID NO: 303 - protein, 9 aa, Mus musculus
  >SEQUENCE_0361 BCR013 IgL CDR3 Cell_013 Mus_musculus
QQSNEDPWT

SEQ ID NO: 304 - protein, 9 aa, Mus musculus
  >SEQUENCE_0362 BCR014 IgL CDR3 Cell_014 Mus_musculus
QQSKEVPYT

SEQ ID NO: 305 - protein, 9 aa, Mus musculus
  >SEQUENCE_0363 BCR015 IgL CDR3 Cell_015 Mus_musculus
ALWYSNHWV

SEQ ID NO: 306 - protein, 9 aa, Mus musculus
  >SEQUENCE_0364 BCR016 IgL CDR3 Cell_016 Mus_musculus
QQSKEVPYT

SEQ ID NO: 307 - protein, 9 aa, Mus musculus
  >SEQUENCE_0365 BCR017 IgL CDR3 Cell_017 Mus_musculus
QQSKEVPWT

SEQ ID NO: 308 - protein, 9 aa, Mus musculus
  >SEQUENCE_0366 BCR018 IgL CDR3 Cell_018 Mus_musculus
QQSKEVPYT

SEQ ID NO: 309 - protein, 9 aa, Mus musculus
  >SEQUENCE_0367 BCR019 IgL CDR3 Cell_019 Mus_musculus
QQSKEVPWT

SEQ ID NO: 310 - protein, 9 aa, Mus musculus
  >SEQUENCE_0368 BCR020 IgL CDR3 Cell_020 Mus_musculus
LQYDDLLRT

SEQ ID NO: 311 - protein, 8 aa, Mus musculus
  >SEQUENCE_0369 BCR021 IgL CDR3 Cell_021 Mus_musculus
HQWSSYPT

SEQ ID NO: 312 - protein, 9 aa, Mus musculus
  >SEQUENCE_0370 BCR022 IgL CDR3 Cell_022 Mus_musculus
QQNNEDPYT

SEQ ID NO: 313 - protein, 9 aa, Mus musculus
  >SEQUENCE_0371 BCR023 IgL CDR3 Cell_023 Mus_musculus
QQSKEVPYT

SEQ ID NO: 314 - protein, 9 aa, Mus musculus
  >SEQUENCE_0372 BCR024 IgL CDR3 Cell_024 Mus_musculus
QQSNEDPYT

SEQ ID NO: 315 - protein, 9 aa, Mus musculus
  >SEQUENCE_0373 BCR025 IgL CDR3 Cell_025 Mus_musculus
ALWYSNLWV

SEQ ID NO: 316 - protein, 9 aa, Mus musculus
  >SEQUENCE_0374 BCR026 IgL CDR3 Cell_026 Mus_musculus
QNDHSYPFT

SEQ ID NO: 317 - protein, 9 aa, Mus musculus
  >SEQUENCE_0375 BCR027 IgL CDR3 Cell_027 Mus_musculus
QQYSGYPYT

SEQ ID NO: 318 - protein, 9 aa, Mus musculus
  >SEQUENCE_0376 BCR028 IgL CDR3 Cell_028 Mus_musculus
QNGHSFPLT

SEQ ID NO: 319 - protein, 9 aa, Mus musculus
  >SEQUENCE_0377 BCR029 IgL CDR3 Cell_029 Mus_musculus
QNGHSFPLT

SEQ ID NO: 320 - protein, 9 aa, Mus musculus
  >SEQUENCE_0378 BCR030 IgL CDR3 Cell_030 Mus_musculus
QQSKEVPLT

SEQ ID NO: 321 - protein, 9 aa, Mus musculus
  >SEQUENCE_0379 BCR031 IgL CDR3 Cell_031 Mus_musculus
QQGQSYPWT

SEQ ID NO: 322 - protein, 9 aa, Mus musculus
  >SEQUENCE_0380 BCR032 IgL CDR3 Cell_032 Mus_musculus
QQSKEVPWT

SEQ ID NO: 323 - protein, 9 aa, Mus musculus
  >SEQUENCE_0381 BCR033 IgL CDR3 Cell_033 Mus_musculus
QQRSSYPLT

SEQ ID NO: 324 - protein, 9 aa, Mus musculus
  >SEQUENCE_0382 BCR034 IgL CDR3 Cell_034 Mus_musculus
QQSKEVPYT

SEQ ID NO: 325 - protein, 9 aa, Mus musculus
  >SEQUENCE_0383 BCR035 IgL CDR3 Cell_035 Mus_musculus
QQSKEVPWT

SEQ ID NO: 326 - protein, 9 aa, Mus musculus
  >SEQUENCE_0384 BCR036 IgL CDR3 Cell_036 Mus_musculus
QQNNEDPLT

SEQ ID NO: 327 - protein, 9 aa, Mus musculus
  >SEQUENCE_0385 BCR037 IgL CDR3 Cell_037 Mus_musculus
HQYHRSPYT

SEQ ID NO: 328 - protein, 11 aa, Mus musculus
  >SEQUENCE_0386 BCR038 IgL CDR3 Cell_038 Mus_musculus
QQFTSSPSPST

SEQ ID NO: 329 - protein, 9 aa, Mus musculus
  >SEQUENCE_0387 BCR039 IgL CDR3 Cell_039 Mus_musculus
QQSKEVPYT

SEQ ID NO: 330 - protein, 9 aa, Mus musculus
  >SEQUENCE_0388 BCR040 IgL CDR3 Cell_040 Mus_musculus
QQSKEVLLT

SEQ ID NO: 331 - protein, 9 aa, Mus musculus
  >SEQUENCE_0389 BCR041 IgL CDR3 Cell_041 Mus_musculus
HQWSSYPYT

SEQ ID NO: 332 - protein, 9 aa, Mus musculus
  >SEQUENCE_0390 BCR042 IgL CDR3 Cell_042 Mus_musculus
QQSKEVPPT

SEQ ID NO: 333 - protein, 9 aa, Mus musculus
  >SEQUENCE_0391 BCR043 IgL CDR3 Cell_043 Mus_musculus
QQSKEVPWT

SEQ ID NO: 334 - protein, 9 aa, Mus musculus
  >SEQUENCE_0392 BCR044 IgL CDR3 Cell_044 Mus_musculus
QQYSKLPWT

SEQ ID NO: 335 - protein, 9 aa, Mus musculus
  >SEQUENCE_0393 BCR045 IgL CDR3 Cell_045 Mus_musculus
QQGQSYPFT

SEQ ID NO: 336 - protein, 9 aa, Mus musculus
  >SEQUENCE_0394 BCR046 IgL CDR3 Cell_046 Mus_musculus
QQRSSYPFT

SEQ ID NO: 337 - protein, 9 aa, Mus musculus
  >SEQUENCE_0395 BCR047 IgL CDR3 Cell_047 Mus_musculus
QQSKEVPWT

SEQ ID NO: 338 - protein, 9 aa, Mus musculus
  >SEQUENCE_0396 BCR048 IgL CDR3 Cell_048 Mus_musculus
QQYSKLPWT

SEQ ID NO: 339 - protein, 9 aa, Mus musculus
  >SEQUENCE_0397 BCR049 IgL CDR3 Cell_049 Mus_musculus
QQYSKLPWT

SEQ ID NO: 340 - protein, 9 aa, Mus musculus
  >SEQUENCE_0398 BCR050 IgL CDR3 Cell_050 Mus_musculus
QQGSSIPRT

SEQ ID NO: 341 - protein, 9 aa, Mus musculus
  >SEQUENCE_0399 BCR051 IgL CDR3 Cell_051 Mus_musculus
QQGSSIPRT

SEQ ID NO: 342 - protein, 9 aa, Mus musculus
  >SEQUENCE_0400 BCR052 IgL CDR3 Cell_052 Mus_musculus
QNGHSFPYT

SEQ ID NO: 343 - protein, 8 aa, Mus musculus
  >SEQUENCE_0401 BCR053 IgL CDR3 Cell_053 Mus_musculus
LQYDNLRT

SEQ ID NO: 344 - protein, 9 aa, Mus musculus
  >SEQUENCE_0402 BCR054 IgL CDR3 Cell_054 Mus_musculus
QQYSKLPWT

SEQ ID NO: 345 - protein, 9 aa, Mus musculus
  >SEQUENCE_0403 BCR055 IgL CDR3 Cell_055 Mus_musculus
QQSKEVPWT

SEQ ID NO: 346 - protein, 9 aa, Mus musculus
  >SEQUENCE_0404 BCR056 IgL CDR3 Cell_056 Mus_musculus
LQYDEFPYT

SEQ ID NO: 347 - protein, 9 aa, Mus musculus
  >SEQUENCE_0405 BCR057 IgL CDR3 Cell_057 Mus_musculus
QQYSKLPWT

SEQ ID NO: 348 - protein, 9 aa, Mus musculus
  >SEQUENCE_0406 BCR058 IgL CDR3 Cell_058 Mus_musculus
QQYSKLPWT

SEQ ID NO: 349 - protein, 106 aa, Mus musculus
  >SEQUENCE_0407 BCR059 IgL Full length Cell_001 Mus_musculus
DIVVTQSPATLSVTPGDRVSLSCRASQSISDYLHWYQQKSHESPRLLIKYASQSISGIPSRFTGSGSGSDFTLTINGVEPEDVGVYYCQNGHTFPFTFGSGQSGNK

SEQ ID NO: 350 - protein, 109 aa, Mus musculus
  >SEQUENCE_0408 BCR060 IgL Full length Cell_002 Mus_musculus
DIVLTQSPASLSVATGEKVTIRCITSTDIDDDMNWYQQKPGEPPKLLISEGNTLRPGVPSRFSSSGYGTDFVFTIENTLSEDVADYYCLQSDNMPYTFGGGTKLEINGD

SEQ ID NO: 351 - protein, 111 aa, Mus musculus
  >SEQUENCE_0409 BCR061 IgL Full length Cell_003 Mus_musculus
DIVLTQSPASLAVSLGQRATISCRASESVESYGNSFIHWYQQKPGQPPKLLMYRASNLESGIPARFSGSGSRTDFTLTINPVEADDVATYFCQQSNEDPWTFGGGTKLEIK

SEQ ID NO: 352 - protein, 111 aa, Mus musculus
  >SEQUENCE_0410 BCR062 IgL Full length Cell_004 Mus_musculus
NIVLTQSPASLAVSLGQRATISCRASESVDSYGNSFMHWYQQKPGQPPKLLIYLASNLESGVPARFSGSGSRTDFTLTIDPVEADDAATYYCQQNNEDPYTFGGGTKLEIN

SEQ ID NO: 353 - protein, 111 aa, Mus musculus
  >SEQUENCE_0411 BCR063 IgL Full length Cell_005 Mus_musculus
DIVMTQSTASLTVSLGQRATVSCRASESVDIYGKSFMHWYQQKPGQPPKLLIYRASNLESGIPARFSGSGSRTDFTLTINPVEADDVATYYCQQSNEDPYTFGGGTKLEIK

SEQ ID NO: 354 - protein, 111 aa, Mus musculus
  >SEQUENCE_0412 BCR064 IgL Full length Cell_006 Mus_musculus
DIVMTQSTASLAVSLGQRATISCRASESVDNYGISFMNWFQQKPGQPPKLLIYAASNQGSGVPARFSGSGSGTDFSLNIHPMEEDDTAMYFCQQSKEVPYTFGGGTKLEIK

SEQ ID NO: 355 - protein, 111 aa, Mus musculus
  >SEQUENCE_0413 BCR065 IgL Full length Cell_007 Mus_musculus
DIVLTQSTASLAVSLGQRATISCRASESVDNFGISFMNWFQQKPGQPPKLLIYAASNQGPGVPARFTGSGSGTDFSLNIHPMEEDDTAMYFCQQSKEVPWTFGGGTKLEIK

SEQ ID NO: 356 - protein, 111 aa, Mus musculus
  >SEQUENCE_0414 BCR066 IgL Full length Cell_008 Mus_musculus
DIVVTQSPASLAVSLGQRATISCRASESVDNFGISFMNWFQQKPGQPPKLLISAASNQGSGVPARFSGSGSGTDFSLNIHPMEEDDTAMYFCQQSKEVPLTFGGGTKLELK

SEQ ID NO: 357 - protein, 107 aa, Mus musculus
  >SEQUENCE_0415 BCR067 IgL Full length Cell_009 Mus_musculus
DIVMTQSPATLSVTPGDRVSLSCRASQSISDYLHWYQQKAHESPRLLIKYASQSISGIPSRFTGSGSGSDFTLTINGVEPEDVGVYYCQNGHTFPFTFGGGTKLELK

SEQ ID NO: 358 - protein, 111 aa, Mus musculus
  >SEQUENCE_0416 BCR068 IgL Full length Cell_010 Mus_musculus
DIVVTQSTVSLAVSLGQRTTISCRASESVDNYGISFMNWFQQKPGQSPKLLIYAASNQGSGVPASFSGSGSGTDFSLNIHPMEEDDTAMYFCQQSKEVPYTFGGGTKLEIK

SEQ ID NO: 359 - protein, 112 aa, Mus musculus
  >SEQUENCE_0417 BCR069 IgL Full length Cell_011 Mus_musculus
DIVMTQSTASLAVSLGQRATISCRASESVDNYGISFMNWFQQKPGQPPKLLIYAASNQGSGVPARFSGSGSGTDFSLNIHPMEEDDTAMYFCQQSKEVPRTFGGGTKLKSNG

SEQ ID NO: 360 - protein, 111 aa, Mus musculus
  >SEQUENCE_0418 BCR070 IgL Full length Cell_012 Mus_musculus
DIVLTQSTASLAVSLGQRATISCRASESVDNYGISFMNWFQQKPGQPPKLLIYAASSQGSGVPARFSGSGSGTDFSLNIHPMEEDDTAMYFCQQSKEVPWTFGGGTKLKSN

SEQ ID NO: 361 - protein, 111 aa, Mus musculus
  >SEQUENCE_0419 BCR071 IgL Full length Cell_013 Mus_musculus
DIVVTQSPASLAVSLGQRATISCRASESVDSYGNSFMHWYQQKPGQPPKLLIYRASNLESGIPARFSGSGSRTDFTLTINPVEADDVATYYCQQSNEDPWTFGGGTKLEIK

SEQ ID NO: 362 - protein, 111 aa, Mus musculus
  >SEQUENCE_0420 BCR072 IgL Full length Cell_014 Mus_musculus
DIVVTQSTVSLAVSLGQRTTISCRASESVDNYGISFMNWFQQKPGQSPKLLIYAASNQGSGVPARFSGSGSGTDFSLNIHPMEEDDTAMYFCQQSKEVPYTFGGGTKLEIK

SEQ ID NO: 363 - protein, 109 aa, Mus musculus
  >SEQUENCE_0421 BCR073 IgL Full length Cell_015 Mus_musculus
QAVVTQESALTTSPGETVTLTCRSSTGAVTTSNHANWVQEKPDHLFTGLIGGTSNRAPGVPVRFSGFLIGDKAALTITGAQTEDEAIYFCALWYSNHWVFGGGTKLTVL

SEQ ID NO: 364 - protein, 111 aa, Mus musculus
  >SEQUENCE_0422 BCR074 IgL Full length Cell_016 Mus_musculus
DIVMTQSTASLAVSLGQRATISCRASESVDNYGISFMNWFQQKPGQPPKLLIYAASNQGSGVPARFRGSGSGTDFSLNIHPMEEDDTAMYFCQQSKEVPYTFGGGTKLEIK

SEQ ID NO: 365 - protein, 111 aa, Mus musculus
  >SEQUENCE_0423 BCR075 IgL Full length Cell_017 Mus_musculus
DIVLTQSTASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 366 - protein, 111 aa, Mus musculus
  >SEQUENCE_0424 BCR076 IgL Full length Cell_018 Mus_musculus
DIVMTQSTASLAVSLGQRATISCRASETVDNYGISFMNWFQQKPGQPPKLLIYAASNQGSGVPARFSGSGSGTDFSLNIHPMEEDDTAMYFCQQSKEVPYTFGGGTKLEIK

SEQ ID NO: 367 - protein, 111 aa, Mus musculus
  >SEQUENCE_0425 BCR077 IgL Full length Cell_019 Mus_musculus
DIVLTQSPASLAVSLGQRATISCRASESVDNFGISFMNWFQQKPGQPPKLLIYAASNQGSGVPARFSGSGSGTDFSLNIHPMEEDDTVMYFCQQSKEVPWTFGGGTKLKSN

SEQ ID NO: 368 - protein, 108 aa, Mus musculus
  >SEQUENCE_0426 BCR078 IgL Full length Cell_020 Mus_musculus
DIVMTQSTSSLSASLGGKVTITSKASQDVNKFIAWYQHKPGKGPWLLIHYTSTLQPGIPSRFSGTGSGRDYSFSISNLEPEDIATYYCLQYDDLLRTFGGGTKLKSDG

SEQ ID NO: 369 - protein, 105 aa, Mus musculus
  >SEQUENCE_0427 BCR079 IgL Full length Cell_021 Mus_musculus
GHCATQSPAIMSAFLGEEITLTCSASSSINYMHWFQQKSGTSPKLLIYSTSNLASGVPSRFSGSGSGTFYSLTISNMEAEDAADYYCHQWSSYPTFGAGTKLEIK

SEQ ID NO: 370 - protein, 111 aa, Mus musculus
  >SEQUENCE_0428 BCR080 IgL Full length Cell_022 Mus_musculus
DIVVTQSPASLAVSLGQRATISCRASESVDSYGKSFMHWYQQKPGQPPKLLIYRASNLESGIPARFSGSGSRTDFTLIIDPVEADDVASYYCQQNNEDPYTFGGGTKLEIK

SEQ ID NO: 371 - protein, 111 aa, Mus musculus
  >SEQUENCE_0429 BCR081 IgL Full length Cell_023 Mus_musculus
DIVLTQSPASLAVSLGQRATISCRASDSVDNYGISFMNWFQQKAGQPPKLLIYAASKQGSGVPARFSGSGSGTDFSLTIHPMEEDDTAMFFCQQSKEVPYTFGGGTKLEIK

SEQ ID NO: 372 - protein, 111 aa, Mus musculus
  >SEQUENCE_0430 BCR082 IgL Full length Cell_024 Mus_musculus
DIVMTQSPASLAVSLWQRATVSCRASESVDRYGKSFMHWYQQKPGQPPKLLIYRASNLESGIPARFSGSGCRTDFTLTINPVEADDVATYYCQQSNEDPYTFGGGTKLEIK

SEQ ID NO: 373 - protein, 114 aa, Mus musculus
  >SEQUENCE_0431 BCR083 IgL Full length Cell_025 Mus_musculus
QAVVTQESALTTSPGETVTLTCRSSTGAVTTSNYANWVQEKPDHLFTGLIGGTNNRVPGVPARFSGSLIGDKAALTITGAQTEDEAIYFCALWYSNLWVFGGGTKLTVLASPFA

SEQ ID NO: 374 - protein, 113 aa, Mus musculus
  >SEQUENCE_0432 BCR084 IgL Full length Cell_026 Mus_musculus
DIVLTQSASSLSVSAGEKVTMGCKSSQSLLNSGNQKNYLAWYQQKPGQPPKLLIYGASTRESGVPDRFTGSGSGTDFTLTISSVQAEDLAVYYCQNDHSYPFTFGSGTKLEIK

SEQ ID NO: 375 - protein, 108 aa, Mus musculus
  >SEQUENCE_0433 BCR085 IgL Full length Cell_027 Mus_musculus
DIVMTQSTAIMSASPGEKVTMTCRASSSVSSSYLHWYQQKSGASPKLWIYSTSNLASGVPARFSGSGSGTSYSLTISSVEAEDAATYYCQQYSGYPYTFGGGTKLEIK

SEQ ID NO: 376 - protein, 107 aa, Mus musculus
  >SEQUENCE_0434 BCR086 IgL Full length Cell_028 Mus_musculus
DIVMTQSPATLSVTPGDRVSLSCRASQSISDYLHWYQQKSHESPRLLIKYASQSISGISSRFTGSGSGSDFTLTINGVEPEDVGVYYCQNGHSFPLTFGAGTKLELK

SEQ ID NO: 377 - protein, 107 aa, Mus musculus
  >SEQUENCE_0435 BCR087 IgL Full length Cell_029 Mus_musculus
DIVMTQSTATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 378 - protein, 111 aa, Mus musculus
  >SEQUENCE_0436 BCR088 IgL Full length Cell_030 Mus_musculus
DNCVTQSPASLAVSLGQRATISCRASESVDNYGISFMNWFQQKPGQPPKLLIYAASNQGSGVPARFSGSGSGTDFSLNIHHMEEDDTAMYFCQQSKEVPLTFGAGTKLELK

SEQ ID NO: 379 - protein, 107 aa, Mus musculus
  >SEQUENCE_0437 BCR089 IgL Full length Cell_031 Mus_musculus
DIQMNQSPSSLSASLGDTITITCHASQNINVWLSWYQQKPGNIPKILIYKASNLHTGVPSRFSGSGSGTGFTLTISSLHPEGIATYYCQQGQSYPWTFAGGTKLETN

SEQ ID NO: 380 - protein, 111 aa, Mus musculus
  >SEQUENCE_0438 BCR090 IgL Full length Cell_032 Mus_musculus
DIVMTQSPASLVVSLGQRASISCRASESVDNFGISFMNWFQQKPGQPPKLLIYAASNQGSGVPARFSGSGSGTDFSLNIHPMEEDDTAMYFCQQSKEVPWTFGGGTKLELK

SEQ ID NO: 381 - protein, 106 aa, Mus musculus
  >SEQUENCE_0439 BCR091 IgL Full length Cell_033 Mus_musculus
QIVLTQSPAIMSASPGEKVTITCSASSSVSDVPWLQQKPGTYPKLWLYSTSNLASGVPARFSGSGSGTSYSLTISRMEAEDAATYYCQQRSSYPLTFGAGTKLELK

SEQ ID NO: 382 - protein, 111 aa, Mus musculus
  >SEQUENCE_0440 BCR092 IgL Full length Cell_034 Mus_musculus
DIVITQTAASLAVSLGQRATISRRASESVDNFGISFMNWFQQKPGQPPKLLIYAASNQGSGVPARFSGSGSGTDFSLNIHPMEEDDTAMYFCQQSKEVPYTFGGGTKLEIK

SEQ ID NO: 383 - protein, 111 aa, Mus musculus
  >SEQUENCE_0441 BCR093 IgL Full length Cell_035 Mus_musculus
DMVMTQSPASLAVSLGQRATISCRVSESVDNYGISFMNWFQLKPGQSPKLLIYAASHQGSGVPARFSGSGSGTDFSLNIHPMEEDDTAMYFCQQSKEVPWTFGGGTKLEIN

SEQ ID NO: 384 - protein, 111 aa, Mus musculus
  >SEQUENCE_0442 BCR094 IgL Full length Cell_036 Mus_musculus
DIVMTQSPASLAVSLGQRATISCRASESVDSYGNSFMHWYQQKPGQPPKLLIYRASNLESGIPARFSGSGSRTDFTLTINPVEADDVATYYCQQNNEDPLTFGAGTKLELK

SEQ ID NO: 385 - protein, 108 aa, Mus musculus
  >SEQUENCE_0443 BCR095 IgL Full length Cell_037 Mus_musculus
QIVLTQSPAIMSASLGERVTMTCTTSSSVSSSYFHWYQQKPGSSPKLWIYSTSNLASGVPTRFSGSGSGTSYSLTISSMETEDAATYYCHQYHRSPYTFGGGTKLEIK

SEQ ID NO: 386 - protein, 108 aa, Mus musculus
  >SEQUENCE_0444 BCR096 IgL Full length Cell_038 Mus_musculus
ENVLTQSPAIMSGSLGEKVTMSCRASSSVNYMYWYQQKSDASPKLWIYYTSNLAPGVPARFSGSGSGNSYSLTISSMEGEDAATYYCQQFTSSPSPSTFGSGTKLEIK

SEQ ID NO: 387 - protein, 111 aa, Mus musculus
  >SEQUENCE_0445 BCR097 IgL Full length Cell_039 Mus_musculus
DIVLTQSPASLAVSLGQRATISCRASESVDNYGISIMNWFQQKPGQPPKLLMYAASNQGSGVPARFSGSGSGTDFSLNIHPMEEDDTAMYFCQQSKEVPYTFGGGTKLEIK

SEQ ID NO: 388 - protein, 111 aa, Mus musculus
  >SEQUENCE_0446 BCR098 IgL Full length Cell_040 Mus_musculus
DIVETQSPASLAVSLGQRATISCRASESVDNYGISFMNWFQQKPGQPPKLLIYAASNQGSGVPARFSGSGSGTDFSLNIHPMEEDDTAMYFCQQSKEVLLTFGGGTKLEIK

SEQ ID NO: 389 - protein, 108 aa, Mus musculus
  >SEQUENCE_0447 BCR099 IgL Full length Cell_041 Mus_musculus
DIVMTQSPAIMSASPGEKVTLTCSASSSISSSYLYWYQQKPGSSPKLWIYSTSNLASGVPARFSGSGSGTSYSLTISNMGAEDAASYFCHQWSSYPYTFGGGTKLEIK

SEQ ID NO: 390 - protein, 111 aa, Mus musculus
  >SEQUENCE_0448 BCR100 IgL Full length Cell_042 Mus_musculus
DIVETQSPASLAVSLGQRATISCRASESVDNYGISFMNWFQQKPGQPPKLLIYAASNQGSGVPARFSGSGSGTDFSLNIHPMEEDDTAMYFCQQSKEVPPTFGGGTKLEIK

SEQ ID NO: 391 - protein, 111 aa, Mus musculus
  >SEQUENCE_0449 BCR101 IgL Full length Cell_043 Mus_musculus
DIVVTQSPASLTVSLGQRATISCRASESVDDYGISFMNWFQQKPGQPPKLLIYIASNQGSGVPARFSGSGSGTDFSLNIHPMEEDDTAMYFCQQSKEVPWTFGGGTKLEIK

SEQ ID NO: 392 - protein, 107 aa, Mus musculus
  >SEQUENCE_0450 BCR102 IgL Full length Cell_044 Mus_musculus
DIVETQSTSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 393 - protein, 107 aa, Mus musculus
  >SEQUENCE_0451 BCR103 IgL Full length Cell_045 Mus_musculus
DICETQSPSSLSASLGDTITITCHASQNINVWLSWYQQKPGNIPKILIYKASNLHTGVPSRFSGSGSGTGFTLTISSLQPEDIATYYCQQGQSYPFTFGSGTKLEMK

SEQ ID NO: 394 - protein, 106 aa, Mus musculus
  >SEQUENCE_0452 BCR104 IgL Full length Cell_046 Mus_musculus
QIVVTQSPAIMSASPGEKVTITCSAGSSVSYMHWFQQKPGTSPKLWIYNTSNLASGVPARFSGSGSGTSYSLTISRMEAEDAATYYCQQRSSYPFTFGSGTKLEIK

SEQ ID NO: 395 - protein, 111 aa, Mus musculus
  >SEQUENCE_0453 BCR105 IgL Full length Cell_047 Mus_musculus
DIVVTQSPASLAVSLGQRATISCRASESVDNYGISFMNWFQQKPGQPPKLLIYVASNQGSGVPARFSGSGSGTDFSLNIHPMEEDDTAMYFCQQSKEVPWTFGGGTKLEIK

SEQ ID NO: 396 - protein, 107 aa, Mus musculus
  >SEQUENCE_0454 BCR106 IgL Full length Cell_048 Mus_musculus
DIQMTQTTSSLSGSLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 397 - protein, 107 aa, Mus musculus
  >SEQUENCE_0455 BCR107 IgL Full length Cell_049 Mus_musculus
DIVMTQTTSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 398 - protein, 108 aa, Mus musculus
  >SEQUENCE_0456 BCR108 IgL Full length Cell_050 Mus_musculus
DIGATQTPTTMAASPGEKITITCSASSSISSNYLHWYQQKPGFSPKLLIYRTSNLASGVPARFSGSGSGTSYSLTIGTMEAEDVATYYCQQGSSIPRTFGSGTKLEIK

SEQ ID NO: 399 - protein, 108 aa, Mus musculus
  >SEQUENCE_0457 BCR109 IgL Full length Cell_051 Mus_musculus
GHCETQSPTTMAASPGEKITITCSASSSISSNYLHWYQQKPGFSPKLLIYRTSNLASGVPARFSGSGSGTSYSLTIGTMEAEDVATYYCQQGSSIPRTFGSGTKLEIK

SEQ ID NO: 400 - protein, 107 aa, Mus musculus
  >SEQUENCE_0458 BCR110 IgL Full length Cell_052 Mus_musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPYTFGGGTKLEIK

SEQ ID NO: 401 - protein, 106 aa, Mus musculus
  >SEQUENCE_0459 BCR111 IgL Full length Cell_053 Mus_musculus
DIVMTQSPSSLSASLGGKVTITCKASQDINNYIAWYQHKPGKGPRLLIHYTFTFQPGIPSRFSGSGSGRDYSFSISNLEPEDIATYYCLQYDNLRTFGGGTKLEIK

SEQ ID NO: 402 - protein, 107 aa, Mus musculus
  >SEQUENCE_0460 BCR112 IgL Full length Cell_054 Mus_musculus
DIVVTQSPSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLEIK

SEQ ID NO: 403 - protein, 109 aa, Mus musculus
  >SEQUENCE_0461 BCR113 IgL Full length Cell_055 Mus_musculus
DIVLTQTPASLTVSLGQRATISCRASESVDDYGISFMSWFQQKPGQPPKLLIYIASNQGSGVPARFSGSGSGTDFSLNIHPMEEDDTAMYFCQQSKEVPWTFGGTKLKS

SEQ ID NO: 404 - protein, 107 aa, Mus musculus
  >SEQUENCE_0462 BCR114 IgL Full length Cell_056 Mus_musculus
GHCETQSPSSMYASLGERVTITCKASQDINSDLSWFQQKPGKSPKTLIYRANRLVDGVPSRFSGSGSGQDYSLTISSLEYEDLGIFYCLQYDEFPYTFGGGTKLEIK

SEQ ID NO: 405 - protein, 107 aa, Mus musculus
  >SEQUENCE_0463 BCR115 IgL Full length Cell_057 Mus_musculus
DIVMTQSPSSLSASLGDRVTISCSASQGISNYLNWYQQKPDGTVKLLIYYTSSLHSGVPSRFSGSGSGTDYSLTISNLEPEDIATYYCQQYSKLPWTFGGGTKLNQT

SEQ ID NO: 406 - protein, 107 aa, Mus musculus
  >SEQUENCE_0464 BCR116 IgL Full length Cell_058 Mus_musculus
DIVMTQSPSSLSASLGDRVTISCSASQGISNFLNWYQQKPDGTVKLLIYYTSSLHSGVPSRFSGSGSGTDCSLTISNLEPEDIATYYCQQYSKLPWTFGGGTKLEIK

SEQ ID NO: 407 - protein, 6 aa, Mus musculus
  >SEQUENCE_0465 TCR001 TRBV CDR1 17 Tcell_001 Mus_musculus
GKSSPN

SEQ ID NO: 408 - protein, 5 aa, Mus musculus
  >SEQUENCE_0466 TCR002 TRBV CDR1 6 Tcell_002 Mus_musculus
SQVVS

SEQ ID NO: 409 - protein, 5 aa, Mus musculus
  >SEQUENCE_0467 TCR003 TRBV CDR1 7 Tcell_003 Mus_musculus
LGHNA

SEQ ID NO: 410 - protein, 5 aa, Mus musculus
  >SEQUENCE_0468 TCR004 TRBV CDR1 4 Tcell_004 Mus_musculus
LGHDT

SEQ ID NO: 411 - protein, 5 aa, Mus musculus
  >SEQUENCE_0469 TCR005 TRBV CDR1 6 Tcell_005 Mus_musculus
SGHSA

SEQ ID NO: 412 - protein, 5 aa, Mus musculus
  >SEQUENCE_0470 TCR006 TRBV CDR1 5 Tcell_006 Mus_musculus
MSHET

SEQ ID NO: 413 - protein, 6 aa, Mus musculus
  >SEQUENCE_0471 TCR007 TRBV CDR1 2 Tcell_007 Mus_musculus
NSQYPW

SEQ ID NO: 414 - protein, 5 aa, Mus musculus
  >SEQUENCE_0472 TCR008 TRBV CDR1 3 Tcell_008 Mus_musculus
NNHDY

SEQ ID NO: 415 - protein, 5 aa, Mus musculus
  >SEQUENCE_0473 TCR009 TRBV CDR1 2 Tcell_009 Mus_musculus
LGHNA

SEQ ID NO: 416 - protein, 5 aa, Mus musculus
  >SEQUENCE_0474 TCR010 TRBV CDR1 2 Tcell_010 Mus_musculus
LGHNA

SEQ ID NO: 417 - protein, 5 aa, Mus musculus
  >SEQUENCE_0475 TCR011 TRBV CDR1 2 Tcell_011 Mus_musculus
SGHSA

SEQ ID NO: 418 - protein, 5 aa, Mus musculus
  >SEQUENCE_0476 TCR012 TRBV CDR1 4 Tcell_012 Mus_musculus
NNHDY

SEQ ID NO: 419 - protein, 5 aa, Mus musculus
  >SEQUENCE_0477 TCR013 TRBV CDR1 2 Tcell_013 Mus_musculus
SNHLY

SEQ ID NO: 420 - protein, 6 aa, Mus musculus
  >SEQUENCE_0478 TCR014 TRBV CDR1 2 Tcell_014 Mus_musculus
GFQATS

SEQ ID NO: 421 - protein, 6 aa, Mus musculus
  >SEQUENCE_0479 TCR015 TRBV CDR1 2 Tcell_015 Mus_musculus
GFQATS

SEQ ID NO: 422 - protein, 5 aa, Mus musculus
  >SEQUENCE_0480 TCR016 TRBV CDR1 2 Tcell_016 Mus_musculus
SGHDT

SEQ ID NO: 423 - protein, 5 aa, Mus musculus
  >SEQUENCE_0481 TCR017 TRBV CDR1 5 Tcell_017 Mus_musculus
SGHLS

SEQ ID NO: 424 - protein, 5 aa, Mus musculus
  >SEQUENCE_0482 TCR018 TRBV CDR1 2 Tcell_018 Mus_musculus
NNHDY

SEQ ID NO: 425 - protein, 5 aa, Mus musculus
  >SEQUENCE_0483 TCR019 TRBV CDR1 2 Tcell_019 Mus_musculus
KGHTA

SEQ ID NO: 426 - protein, 5 aa, Mus musculus
  >SEQUENCE_0484 TCR020 TRBV CDR1 10 Tcell_020 Mus_musculus
NNHDY

SEQ ID NO: 427 - protein, 5 aa, Mus musculus
  >SEQUENCE_0485 TCR021 TRBV CDR1 3 Tcell_021 Mus_musculus
FNHDT

SEQ ID NO: 428 - protein, 5 aa, Mus musculus
  >SEQUENCE_0486 TCR022 TRBV CDR1 3 Tcell_022 Mus_musculus
SGHDT

SEQ ID NO: 429 - protein, 5 aa, Mus musculus
  >SEQUENCE_0487 TCR023 TRBV CDR1 3 Tcell_023 Mus_musculus
NNHDY

SEQ ID NO: 430 - protein, 6 aa, Mus musculus
  >SEQUENCE_0488 TCR024 TRBV CDR1 3 Tcell_024 Mus_musculus
GKSSPN

SEQ ID NO: 431 - protein, 6 aa, Mus musculus
  >SEQUENCE_0489 TCR025 TRBV CDR1 2 Tcell_025 Mus_musculus
GKSSPN

SEQ ID NO: 432 - protein, 5 aa, Mus musculus
  >SEQUENCE_0490 TCR026 TRBV CDR1 2 Tcell_026 Mus_musculus
FNHDT

SEQ ID NO: 433 - protein, 5 aa, Mus musculus
  >SEQUENCE_0491 TCR027 TRBV CDR1 2 Tcell_027 Mus_musculus
MNHDT

SEQ ID NO: 434 - protein, 6 aa, Mus musculus
  >SEQUENCE_0492 TCR028 TRBV CDR1 2 Tcell_028 Mus_musculus
GFQATS

SEQ ID NO: 435 - protein, 6 aa, Mus musculus
  >SEQUENCE_0493 TCR029 TRBV CDR1 2 Tcell_029 Mus_musculus
NSQYPW

SEQ ID NO: 436 - protein, 5 aa, Mus musculus
  >SEQUENCE_0494 TCR030 TRBV CDR1 2 Tcell_030 Mus_musculus
SQVVS

SEQ ID NO: 437 - protein, 5 aa, Mus musculus
  >SEQUENCE_0495 TCR031 TRBV CDR1 2 Tcell_031 Mus_musculus
NNHNN

SEQ ID NO: 438 - protein, 5 aa, Mus musculus
  >SEQUENCE_0496 TCR032 TRBV CDR1 2 Tcell_032 Mus_musculus
SGHLS

SEQ ID NO: 439 - protein, 5 aa, Mus musculus
  >SEQUENCE_0497 TCR033 TRBV CDR1 2 Tcell_033 Mus_musculus
LGHNA

SEQ ID NO: 440 - protein, 5 aa, Mus musculus
  >SEQUENCE_0498 TCR034 TRBV CDR1 2 Tcell_034 Mus_musculus
LGHNA

SEQ ID NO: 441 - protein, 5 aa, Mus musculus
  >SEQUENCE_0499 TCR035 TRBV CDR1 2 Tcell_035 Mus_musculus
KGHPV

SEQ ID NO: 442 - protein, 6 aa, Mus musculus
  >SEQUENCE_0500 TCR036 TRBV CDR1 2 Tcell_036 Mus_musculus
NSQYPW

SEQ ID NO: 443 - protein, 5 aa, Mus musculus
  >SEQUENCE_0501 TCR037 TRBV CDR1 2 Tcell_037 Mus_musculus
NNHNN

SEQ ID NO: 444 - protein, 5 aa, Mus musculus
  >SEQUENCE_0502 TCR038 TRBV CDR1 4 Tcell_038 Mus_musculus
SGHDT

SEQ ID NO: 445 - protein, 5 aa, Mus musculus
  >SEQUENCE_0503 TCR039 TRBV CDR1 12 Tcell_039 Mus_musculus
SGHSA

SEQ ID NO: 446 - protein, 5 aa, Mus musculus
  >SEQUENCE_0504 TCR040 TRBV CDR1 5 Tcell_040 Mus_musculus
FNHDT

SEQ ID NO: 447 - protein, 5 aa, Mus musculus
  >SEQUENCE_0505 TCR041 TRBV CDR1 4 Tcell_041 Mus_musculus
SGHLS

SEQ ID NO: 448 - protein, 5 aa, Mus musculus
  >SEQUENCE_0506 TCR042 TRBV CDR1 4 Tcell_042 Mus_musculus
SNHLY

SEQ ID NO: 449 - protein, 6 aa, Mus musculus
  >SEQUENCE_0507 TCR043 TRBV CDR1 3 Tcell_043 Mus_musculus
GFQATS

SEQ ID NO: 450 - protein, 5 aa, Mus musculus
  >SEQUENCE_0508 TCR044 TRBV CDR1 5 Tcell_044 Mus_musculus
NSHNY

SEQ ID NO: 451 - protein, 5 aa, Mus musculus
  >SEQUENCE_0509 TCR045 TRBV CDR1 3 Tcell_045 Mus_musculus
NSHNY

SEQ ID NO: 452 - protein, 5 aa, Mus musculus
  >SEQUENCE_0510 TCR046 TRBV CDR1 3 Tcell_046 Mus_musculus
NNHDY

SEQ ID NO: 453 - protein, 5 aa, Mus musculus
  >SEQUENCE_0511 TCR047 TRBV CDR1 3 Tcell_047 Mus_musculus
NSHNY

SEQ ID NO: 454 - protein, 5 aa, Mus musculus
  >SEQUENCE_0512 TCR048 TRBV CDR1 3 Tcell_048 Mus_musculus
LGHNA

SEQ ID NO: 455 - protein, 6 aa, Mus musculus
  >SEQUENCE_0513 TCR049 TRBV CDR1 3 Tcell_049 Mus_musculus
GFQATS

SEQ ID NO: 456 - protein, 5 aa, Mus musculus
  >SEQUENCE_0514 TCR050 TRBV CDR1 3 Tcell_050 Mus_musculus
NSHNY

SEQ ID NO: 457 - protein, 5 aa, Mus musculus
  >SEQUENCE_0515 TCR051 TRBV CDR1 3 Tcell_051 Mus_musculus
KGHPV

SEQ ID NO: 458 - protein, 5 aa, Mus musculus
  >SEQUENCE_0516 TCR052 TRBV CDR1 3 Tcell_052 Mus_musculus
KGHPV

SEQ ID NO: 459 - protein, 5 aa, Mus musculus
  >SEQUENCE_0517 TCR053 TRBV CDR1 3 Tcell_053 Mus_musculus
NNHNN

SEQ ID NO: 460 - protein, 5 aa, Mus musculus
  >SEQUENCE_0518 TCR054 TRBV CDR1 3 Tcell_054 Mus_musculus
NNHNN

SEQ ID NO: 461 - protein, 5 aa, Mus musculus
  >SEQUENCE_0519 TCR055 TRBV CDR1 3 Tcell_055 Mus_musculus
SGHLS

SEQ ID NO: 462 - protein, 5 aa, Mus musculus
  >SEQUENCE_0520 TCR056 TRBV CDR1 2 Tcell_056 Mus_musculus
SNHLY

SEQ ID NO: 463 - protein, 5 aa, Mus musculus
  >SEQUENCE_0521 TCR057 TRBV CDR1 2 Tcell_057 Mus_musculus
NSHNY

SEQ ID NO: 464 - protein, 5 aa, Mus musculus
  >SEQUENCE_0522 TCR058 TRBV CDR1 2 Tcell_058 Mus_musculus
NNHNN

SEQ ID NO: 465 - protein, 5 aa, Mus musculus
  >SEQUENCE_0523 TCR059 TRBV CDR1 2 Tcell_059 Mus_musculus
KGHPV

SEQ ID NO: 466 - protein, 5 aa, Mus musculus
  >SEQUENCE_0524 TCR060 TRBV CDR1 2 Tcell_060 Mus_musculus
LGHNA

SEQ ID NO: 467 - protein, 5 aa, Mus musculus
  >SEQUENCE_0525 TCR061 TRBV CDR1 2 Tcell_061 Mus_musculus
LGHDT

SEQ ID NO: 468 - protein, 5 aa, Mus musculus
  >SEQUENCE_0526 TCR062 TRBV CDR1 2 Tcell_062 Mus_musculus
FNHDT

SEQ ID NO: 469 - protein, 5 aa, Mus musculus
  >SEQUENCE_0527 TCR063 TRBV CDR1 2 Tcell_063 Mus_musculus
NNHNN

SEQ ID NO: 470 - protein, 5 aa, Mus musculus
  >SEQUENCE_0528 TCR064 TRBV CDR1 2 Tcell_064 Mus_musculus
NNHNN

SEQ ID NO: 471 - protein, 5 aa, Mus musculus
  >SEQUENCE_0529 TCR065 TRBV CDR1 2 Tcell_065 Mus_musculus
FNHDT

SEQ ID NO: 472 - protein, 5 aa, Mus musculus
  >SEQUENCE_0530 TCR066 TRBV CDR1 2 Tcell_066 Mus_musculus
FNHDT

SEQ ID NO: 473 - protein, 5 aa, Mus musculus
  >SEQUENCE_0531 TCR067 TRBV CDR1 2 Tcell_067 Mus_musculus
NNHNN

SEQ ID NO: 474 - protein, 5 aa, Mus musculus
  >SEQUENCE_0532 TCR068 TRBV CDR1 2 Tcell_068 Mus_musculus
FNHDT

SEQ ID NO: 475 - protein, 5 aa, Mus musculus
  >SEQUENCE_0533 TCR069 TRBV CDR1 2 Tcell_069 Mus_musculus
MNHDT

SEQ ID NO: 476 - protein, 5 aa, Mus musculus
  >SEQUENCE_0534 TCR070 TRBV CDR1 2 Tcell_070 Mus_musculus
LGHNA

SEQ ID NO: 477 - protein, 5 aa, Mus musculus
  >SEQUENCE_0535 TCR071 TRBV CDR1 2 Tcell_071 Mus_musculus
SNHLY

SEQ ID NO: 478 - protein, 5 aa, Mus musculus
  >SEQUENCE_0536 TCR001 TRBV CDR2 17 Tcell_001 Mus_musculus
SITVG

SEQ ID NO: 479 - protein, 7 aa, Mus musculus
  >SEQUENCE_0537 TCR002 TRBV CDR2 6 Tcell_002 Mus_musculus
ANEGSEA

SEQ ID NO: 480 - protein, 6 aa, Mus musculus
  >SEQUENCE_0538 TCR003 TRBV CDR2 7 Tcell_003 Mus_musculus
YNLKQL

SEQ ID NO: 481 - protein, 6 aa, Mus musculus
  >SEQUENCE_0539 TCR004 TRBV CDR2 4 Tcell_004 Mus_musculus
YNNKQL

SEQ ID NO: 482 - protein, 6 aa, Mus musculus
  >SEQUENCE_0540 TCR005 TRBV CDR2 6 Tcell_005 Mus_musculus
FRNQAP

SEQ ID NO: 483 - protein, 6 aa, Mus musculus
  >SEQUENCE_0541 TCR006 TRBV CDR2 5 Tcell_006 Mus_musculus
SYDVDS

SEQ ID NO: 484 - protein, 6 aa, Mus musculus
  >SEQUENCE_0542 TCR007 TRBV CDR2 2 Tcell_007 Mus_musculus
LRSPGD

SEQ ID NO: 485 - protein, 6 aa, Mus musculus
  >SEQUENCE_0543 TCR008 TRBV CDR2 3 Tcell_008 Mus_musculus
SYVADS

SEQ ID NO: 486 - protein, 6 aa, Mus musculus
  >SEQUENCE_0544 TCR009 TRBV CDR2 2 Tcell_009 Mus_musculus
YNLKQL

SEQ ID NO: 487 - protein, 6 aa, Mus musculus
  >SEQUENCE_0545 TCR010 TRBV CDR2 2 Tcell_010 Mus_musculus
YNLKQL

SEQ ID NO: 488 - protein, 6 aa, Mus musculus
  >SEQUENCE_0546 TCR011 TRBV CDR2 2 Tcell_011 Mus_musculus
FRNQAP

SEQ ID NO: 489 - protein, 6 aa, Mus musculus
  >SEQUENCE_0547 TCR012 TRBV CDR2 4 Tcell_012 Mus_musculus
SYVADS

SEQ ID NO: 490 - protein, 6 aa, Mus musculus
  >SEQUENCE_0548 TCR013 TRBV CDR2 2 Tcell_013 Mus_musculus
FYNGKV

SEQ ID NO: 491 - protein, 7 aa, Mus musculus
  >SEQUENCE_0549 TCR014 TRBV CDR2 2 Tcell_014 Mus_musculus
STVNSAI

SEQ ID NO: 492 - protein, 7 aa, Mus musculus
  >SEQUENCE_0550 TCR015 TRBV CDR2 2 Tcell_015 Mus_musculus
STVNSAI

SEQ ID NO: 493 - protein, 6 aa, Mus musculus
  >SEQUENCE_0551 TCR016 TRBV CDR2 2 Tcell_016 Mus_musculus
FRDEAV

SEQ ID NO: 494 - protein, 6 aa, Mus musculus
  >SEQUENCE_0552 TCR017 TRBV CDR2 5 Tcell_017 Mus_musculus
HYDKME

SEQ ID NO: 495 - protein, 6 aa, Mus musculus
  >SEQUENCE_0553 TCR018 TRBV CDR2 2 Tcell_018 Mus_musculus
SYVADS

SEQ ID NO: 496 - protein, 6 aa, Mus musculus
  >SEQUENCE_0554 TCR019 TRBV CDR2 2 Tcell_019 Mus_musculus
FQNQQP

SEQ ID NO: 497 - protein, 6 aa, Mus musculus
  >SEQUENCE_0555 TCR020 TRBV CDR2 10 Tcell_020 Mus_musculus
SYVADS

SEQ ID NO: 498 - protein, 6 aa, Mus musculus
  >SEQUENCE_0556 TCR021 TRBV CDR2 3 Tcell_021 Mus_musculus
SITEND

SEQ ID NO: 499 - protein, 6 aa, Mus musculus
  >SEQUENCE_0557 TCR022 TRBV CDR2 3 Tcell_022 Mus_musculus
FRDEAV

SEQ ID NO: 500 - protein, 6 aa, Mus musculus
  >SEQUENCE_0558 TCR023 TRBV CDR2 3 Tcell_023 Mus_musculus
SYVADS

SEQ ID NO: 501 - protein, 5 aa, Mus musculus
  >SEQUENCE_0559 TCR024 TRBV CDR2 3 Tcell_024 Mus_musculus
SITVG

SEQ ID NO: 502 - protein, 5 aa, Mus musculus
  >SEQUENCE_0560 TCR025 TRBV CDR2 2 Tcell_025 Mus_musculus
SITVG

SEQ ID NO: 503 - protein, 6 aa, Mus musculus
  >SEQUENCE_0561 TCR026 TRBV CDR2 2 Tcell_026 Mus_musculus
SITEND

SEQ ID NO: 504 - protein, 6 aa, Mus musculus
  >SEQUENCE_0562 TCR027 TRBV CDR2 2 Tcell_027 Mus_musculus
YYDKIL

SEQ ID NO: 505 - protein, 7 aa, Mus musculus
  >SEQUENCE_0563 TCR028 TRBV CDR2 2 Tcell_028 Mus_musculus
STVNSAI

SEQ ID NO: 506 - protein, 6 aa, Mus musculus
  >SEQUENCE_0564 TCR029 TRBV CDR2 2 Tcell_029 Mus_musculus
LRSPGD

SEQ ID NO: 507 - protein, 7 aa, Mus musculus
  >SEQUENCE_0565 TCR030 TRBV CDR2 2 Tcell_030 Mus_musculus
ANEGSEA

SEQ ID NO: 508 - protein, 6 aa, Mus musculus
  >SEQUENCE_0566 TCR031 TRBV CDR2 2 Tcell_031 Mus_musculus
SYGAGS

SEQ ID NO: 509 - protein, 6 aa, Mus musculus
  >SEQUENCE_0567 TCR032 TRBV CDR2 2 Tcell_032 Mus_musculus
HYDKME

SEQ ID NO: 510 - protein, 6 aa, Mus musculus
  >SEQUENCE_0568 TCR033 TRBV CDR2 2 Tcell_033 Mus_musculus
YNLKQL

SEQ ID NO: 511 - protein, 6 aa, Mus musculus
  >SEQUENCE_0569 TCR034 TRBV CDR2 2 Tcell_034 Mus_musculus
YSYQKL

SEQ ID NO: 512 - protein, 6 aa, Mus musculus
  >SEQUENCE_0570 TCR035 TRBV CDR2 2 Tcell_035 Mus_musculus
FQNQEV

SEQ ID NO: 513 - protein, 6 aa, Mus musculus
  >SEQUENCE_0571 TCR036 TRBV CDR2 2 Tcell_036 Mus_musculus
LRSPGD

SEQ ID NO: 514 - protein, 6 aa, Mus musculus
  >SEQUENCE_0572 TCR037 TRBV CDR2 2 Tcell_037 Mus_musculus
SYGAGR

SEQ ID NO: 515 - protein, 6 aa, Mus musculus
  >SEQUENCE_0573 TCR038 TRBV CDR2 4 Tcell_038 Mus_musculus
FRDEAV

SEQ ID NO: 516 - protein, 6 aa, Mus musculus
  >SEQUENCE_0574 TCR039 TRBV CDR2 12 Tcell_039 Mus_musculus
FRNQAP

SEQ ID NO: 517 - protein, 6 aa, Mus musculus
  >SEQUENCE_0575 TCR040 TRBV CDR2 5 Tcell_040 Mus_musculus
SITEND

SEQ ID NO: 518 - protein, 6 aa, Mus musculus
  >SEQUENCE_0576 TCR041 TRBV CDR2 4 Tcell_041 Mus_musculus
HYDKME

SEQ ID NO: 519 - protein, 6 aa, Mus musculus
  >SEQUENCE_0577 TCR042 TRBV CDR2 4 Tcell_042 Mus_musculus
FYNGKV

SEQ ID NO: 520 - protein, 7 aa, Mus musculus
  >SEQUENCE_0578 TCR043 TRBV CDR2 3 Tcell_043 Mus_musculus
STVNSAI

SEQ ID NO: 521 - protein, 6 aa, Mus musculus
  >SEQUENCE_0579 TCR044 TRBV CDR2 5 Tcell_044 Mus_musculus
SYGAGN

SEQ ID NO: 522 - protein, 6 aa, Mus musculus
  >SEQUENCE_0580 TCR045 TRBV CDR2 3 Tcell_045 Mus_musculus
SYGAGN

SEQ ID NO: 523 - protein, 6 aa, Mus musculus
  >SEQUENCE_0581 TCR046 TRBV CDR2 3 Tcell_046 Mus_musculus
SYVADS

SEQ ID NO: 524 - protein, 6 aa, Mus musculus
  >SEQUENCE_0582 TCR047 TRBV CDR2 3 Tcell_047 Mus_musculus
SYGAGN

SEQ ID NO: 525 - protein, 6 aa, Mus musculus
  >SEQUENCE_0583 TCR048 TRBV CDR2 3 Tcell_048 Mus_musculus
YNLKQL

SEQ ID NO: 526 - protein, 7 aa, Mus musculus
  >SEQUENCE_0584 TCR049 TRBV CDR2 3 Tcell_049 Mus_musculus
STVNSAI

SEQ ID NO: 527 - protein, 6 aa, Mus musculus
  >SEQUENCE_0585 TCR050 TRBV CDR2 3 Tcell_050 Mus_musculus
SYGAGN

SEQ ID NO: 528 - protein, 6 aa, Mus musculus
  >SEQUENCE_0586 TCR051 TRBV CDR2 3 Tcell_051 Mus_musculus
FQNQEV

SEQ ID NO: 529 - protein, 6 aa, Mus musculus
  >SEQUENCE_0587 TCR052 TRBV CDR2 3 Tcell_052 Mus_musculus
FQNQEV

SEQ ID NO: 530 - protein, 6 aa, Mus musculus
  >SEQUENCE_0588 TCR053 TRBV CDR2 3 Tcell_053 Mus_musculus
SYGAGS

SEQ ID NO: 531 - protein, 6 aa, Mus musculus
  >SEQUENCE_0589 TCR054 TRBV CDR2 3 Tcell_054 Mus_musculus
SYGAGS

SEQ ID NO: 532 - protein, 6 aa, Mus musculus
  >SEQUENCE_0590 TCR055 TRBV CDR2 3 Tcell_055 Mus_musculus
HYDKME

SEQ ID NO: 533 - protein, 6 aa, Mus musculus
  >SEQUENCE_0591 TCR056 TRBV CDR2 2 Tcell_056 Mus_musculus
FYNGKV

SEQ ID NO: 534 - protein, 6 aa, Mus musculus
  >SEQUENCE_0592 TCR057 TRBV CDR2 2 Tcell_057 Mus_musculus
SYGAGN

SEQ ID NO: 535 - protein, 6 aa, Mus musculus
  >SEQUENCE_0593 TCR058 TRBV CDR2 2 Tcell_058 Mus_musculus
SYGAGS

SEQ ID NO: 536 - protein, 6 aa, Mus musculus
  >SEQUENCE_0594 TCR059 TRBV CDR2 2 Tcell_059 Mus_musculus
FQNQEV

SEQ ID NO: 537 - protein, 6 aa, Mus musculus
  >SEQUENCE_0595 TCR060 TRBV CDR2 2 Tcell_060 Mus_musculus
YNLKQL

SEQ ID NO: 538 - protein, 6 aa, Mus musculus
  >SEQUENCE_0596 TCR061 TRBV CDR2 2 Tcell_061 Mus_musculus
YNNKQL

SEQ ID NO: 539 - protein, 6 aa, Mus musculus
  >SEQUENCE_0597 TCR062 TRBV CDR2 2 Tcell_062 Mus_musculus
SITEND

SEQ ID NO: 540 - protein, 6 aa, Mus musculus
  >SEQUENCE_0598 TCR063 TRBV CDR2 2 Tcell_063 Mus_musculus
SYGAGS

SEQ ID NO: 541 - protein, 6 aa, Mus musculus
  >SEQUENCE_0599 TCR064 TRBV CDR2 2 Tcell_064 Mus_musculus
SYVADS

SEQ ID NO: 542 - protein, 6 aa, Mus musculus
  >SEQUENCE_0600 TCR065 TRBV CDR2 2 Tcell_065 Mus_musculus
SITEND

SEQ ID NO: 543 - protein, 6 aa, Mus musculus
  >SEQUENCE_0601 TCR066 TRBV CDR2 2 Tcell_066 Mus_musculus
SITEND

SEQ ID NO: 544 - protein, 6 aa, Mus musculus
  >SEQUENCE_0602 TCR067 TRBV CDR2 2 Tcell_067 Mus_musculus
SYGAGS

SEQ ID NO: 545 - protein, 6 aa, Mus musculus
  >SEQUENCE_0603 TCR068 TRBV CDR2 2 Tcell_068 Mus_musculus
SITEND

SEQ ID NO: 546 - protein, 6 aa, Mus musculus
  >SEQUENCE_0604 TCR069 TRBV CDR2 2 Tcell_069 Mus_musculus
YYDKIL

SEQ ID NO: 547 - protein, 6 aa, Mus musculus
  >SEQUENCE_0605 TCR070 TRBV CDR2 2 Tcell_070 Mus_musculus
YNLKQL

SEQ ID NO: 548 - protein, 6 aa, Mus musculus
  >SEQUENCE_0606 TCR071 TRBV CDR2 2 Tcell_071 Mus_musculus
FYNGKV

SEQ ID NO: 549 - protein, 15 aa, Mus musculus
  >SEQUENCE_0607 TCR001 TRBV CDR3 17 Tcell_001 Mus_musculus
CAWSPQGTSNTEVFF

SEQ ID NO: 550 - protein, 11 aa, Mus musculus
  >SEQUENCE_0608 TCR002 TRBV CDR3 6 Tcell_002 Mus_musculus
CSSIHLYEQNF

SEQ ID NO: 551 - protein, 13 aa, Mus musculus
  >SEQUENCE_0609 TCR003 TRBV CDR3 7 Tcell_003 Mus_musculus
CASSQGDWREQYF

SEQ ID NO: 552 - protein, 16 aa, Mus musculus
  >SEQUENCE_0610 TCR004 TRBV CDR3 4 Tcell_004 Mus_musculus
CASSELGGRGQNTLYF

SEQ ID NO: 553 - protein, 11 aa, Mus musculus
  >SEQUENCE_0611 TCR005 TRBV CDR3 6 Tcell_005 Mus_musculus
CASSLTVEQYF

SEQ ID NO: 554 - protein, 15 aa, Mus musculus
  >SEQUENCE_0612 TCR006 TRBV CDR3 5 Tcell_006 Mus_musculus
CASSPGQGANTEVFF

SEQ ID NO: 555 - protein, 14 aa, Mus musculus
  >SEQUENCE_0613 TCR007 TRBV CDR3 2 Tcell_007 Mus_musculus
CASSVGQSSYEQYF

SEQ ID NO: 556 - protein, 14 aa, Mus musculus
  >SEQUENCE_0614 TCR008 TRBV CDR3 3 Tcell_008 Mus_musculus
CASSVGQSSYEQYF

SEQ ID NO: 557 - protein, 14 aa, Mus musculus
  >SEQUENCE_0615 TCR009 TRBV CDR3 2 Tcell_009 Mus_musculus
CASSQDWQQDTQYF

SEQ ID NO: 558 - protein, 15 aa, Mus musculus
  >SEQUENCE_0616 TCR010 TRBV CDR3 2 Tcell_010 Mus_musculus
CASSQVPGQLYEQYF

SEQ ID NO: 559 - protein, 13 aa, Mus musculus
  >SEQUENCE_0617 TCR011 TRBV CDR3 2 Tcell_011 Mus_musculus
CASSPWTVYEQYF

SEQ ID NO: 560 - protein, 17 aa, Mus musculus
  >SEQUENCE_0618 TCR012 TRBV CDR3 4 Tcell_012 Mus_musculus
CASSDEDWGASAETLYF

SEQ ID NO: 561 - protein, 15 aa, Mus musculus
  >SEQUENCE_0619 TCR013 TRBV CDR3 2 Tcell_013 Mus_musculus
CASSSGRRSGNTLYF

SEQ ID NO: 562 - protein, 14 aa, Mus musculus
  >SEQUENCE_0620 TCR014 TRBV CDR3 2 Tcell_014 Mus_musculus
CGAFDRMSAETLYF

SEQ ID NO: 563 - protein, 13 aa, Mus musculus
  >SEQUENCE_0621 TCR015 TRBV CDR3 2 Tcell_015 Mus_musculus
CGAREGTNSPLYF

SEQ ID NO: 564 - protein, 14 aa, Mus musculus
  >SEQUENCE_0622 TCR016 TRBV CDR3 2 Tcell_016 Mus_musculus
CASSFRLGGREQYF

SEQ ID NO: 565 - protein, 14 aa, Mus musculus
  >SEQUENCE_0623 TCR017 TRBV CDR3 5 Tcell_017 Mus_musculus
CASSLGGRSYEQYF

SEQ ID NO: 566 - protein, 14 aa, Mus musculus
  >SEQUENCE_0624 TCR018 TRBV CDR3 2 Tcell_018 Mus_musculus
CASSDRGQAETLYF

SEQ ID NO: 567 - protein, 12 aa, Mus musculus
  >SEQUENCE_0625 TCR019 TRBV CDR3 2 Tcell_019 Mus_musculus
CSSSLEQDTQYF

SEQ ID NO: 568 - protein, 13 aa, Mus musculus
  >SEQUENCE_0626 TCR020 TRBV CDR3 10 Tcell_020 Mus_musculus
CASSNRGRDTQYF

SEQ ID NO: 569 - protein, 15 aa, Mus musculus
  >SEQUENCE_0627 TCR021 TRBV CDR3 3 Tcell_021 Mus_musculus
CASSPLDREDTEVFF

SEQ ID NO: 570 - protein, 14 aa, Mus musculus
  >SEQUENCE_0628 TCR022 TRBV CDR3 3 Tcell_022 Mus_musculus
CASSLGDSAETLYF

SEQ ID NO: 571 - protein, 14 aa, Mus musculus
  >SEQUENCE_0629 TCR023 TRBV CDR3 3 Tcell_023 Mus_musculus
CASSDRASNTEVFF

SEQ ID NO: 572 - protein, 16 aa, Mus musculus
  >SEQUENCE_0630 TCR024 TRBV CDR3 3 Tcell_024 Mus_musculus
CAWSPPTGVSAETLYF

SEQ ID NO: 573 - protein, 14 aa, Mus musculus
  >SEQUENCE_0631 TCR025 TRBV CDR3 2 Tcell_025 Mus_musculus
CAWSPGLGGREQYF

SEQ ID NO: 574 - protein, 14 aa, Mus musculus
  >SEQUENCE_0632 TCR026 TRBV CDR3 2 Tcell_026 Mus_musculus
CASTQGATNERLFF

SEQ ID NO: 575 - protein, 12 aa, Mus musculus
  >SEQUENCE_0633 TCR027 TRBV CDR3 2 Tcell_027 Mus_musculus
CASSRNRDTLYF

SEQ ID NO: 576 - protein, 14 aa, Mus musculus
  >SEQUENCE_0634 TCR028 TRBV CDR3 2 Tcell_028 Mus_musculus
CGASRGQTYAEQFF

SEQ ID NO: 577 - protein, 12 aa, Mus musculus
  >SEQUENCE_0635 TCR029 TRBV CDR3 2 Tcell_029 Mus_musculus
CSWDSSNERLFF

SEQ ID NO: 578 - protein, 11 aa, Mus musculus
  >SEQUENCE_0636 TCR030 TRBV CDR3 2 Tcell_030 Mus_musculus
CSSIHLYEQYF

SEQ ID NO: 579 - protein, 13 aa, Mus musculus
  >SEQUENCE_0637 TCR031 TRBV CDR3 2 Tcell_031 Mus_musculus
CASGASGNQAPLF

SEQ ID NO: 580 - protein, 13 aa, Mus musculus
  >SEQUENCE_0638 TCR032 TRBV CDR3 2 Tcell_032 Mus_musculus
CASSLGTQDTQYF

SEQ ID NO: 581 - protein, 15 aa, Mus musculus
  >SEQUENCE_0639 TCR033 TRBV CDR3 2 Tcell_033 Mus_musculus
CASSQGLGVSYEQYF

SEQ ID NO: 582 - protein, 11 aa, Mus musculus
  >SEQUENCE_0640 TCR034 TRBV CDR3 2 Tcell_034 Mus_musculus
CASSQESQVFF

SEQ ID NO: 583 - protein, 13 aa, Mus musculus
  >SEQUENCE_0641 TCR035 TRBV CDR3 2 Tcell_035 Mus_musculus
CASSLTTNSDYTF

SEQ ID NO: 584 - protein, 14 aa, Mus musculus
  >SEQUENCE_0642 TCR036 TRBV CDR3 2 Tcell_036 Mus_musculus
CSADWGANTGQLYF

SEQ ID NO: 585 - protein, 16 aa, Mus musculus
  >SEQUENCE_0643 TCR037 TRBV CDR3 2 Tcell_037 Mus_musculus
CASGDALGGGQNTLYF

SEQ ID NO: 586 - protein, 14 aa, Mus musculus
  >SEQUENCE_0644 TCR038 TRBV CDR3 4 Tcell_038 Mus_musculus
CASSFLGRGDTQYF

SEQ ID NO: 587 - protein, 15 aa, Mus musculus
  >SEQUENCE_0645 TCR039 TRBV CDR3 12 Tcell_039 Mus_musculus
CASSPDWGSSYEQYF

SEQ ID NO: 588 - protein, 13 aa, Mus musculus
  >SEQUENCE_0646 TCR040 TRBV CDR3 5 Tcell_040 Mus_musculus
CASRDREDERLFF

SEQ ID NO: 589 - protein, 14 aa, Mus musculus
  >SEQUENCE_0647 TCR041 TRBV CDR3 4 Tcell_041 Mus_musculus
CASSLDGSNERLFF

SEQ ID NO: 590 - protein, 14 aa, Mus musculus
  >SEQUENCE_0648 TCR042 TRBV CDR3 4 Tcell_042 Mus_musculus
CASSPGQGNTEVFF

SEQ ID NO: 591 - protein, 14 aa, Mus musculus
  >SEQUENCE_0649 TCR043 TRBV CDR3 3 Tcell_043 Mus_musculus
CGASRGQTYAEQFF

SEQ ID NO: 592 - protein, 13 aa, Mus musculus
  >SEQUENCE_0650 TCR044 TRBV CDR3 5 Tcell_044 Mus_musculus
CASSGTGVYEQYF

SEQ ID NO: 593 - protein, 13 aa, Mus musculus
  >SEQUENCE_0651 TCR045 TRBV CDR3 3 Tcell_045 Mus_musculus
CASSGTGVYEQYF

SEQ ID NO: 594 - protein, 14 aa, Mus musculus
  >SEQUENCE_0652 TCR046 TRBV CDR3 3 Tcell_046 Mus_musculus
CASSALGGQDTQYF

SEQ ID NO: 595 - protein, 14 aa, Mus musculus
  >SEQUENCE_0653 TCR047 TRBV CDR3 3 Tcell_047 Mus_musculus
CASSLGGGQDTQYF

SEQ ID NO: 596 - protein, 15 aa, Mus musculus
  >SEQUENCE_0654 TCR048 TRBV CDR3 3 Tcell_048 Mus_musculus
CASSQVGAGNQAPLF

SEQ ID NO: 597 - protein, 12 aa, Mus musculus
  >SEQUENCE_0655 TCR049 TRBV CDR3 3 Tcell_049 Mus_musculus
CGASTQNSDYTF

SEQ ID NO: 598 - protein, 14 aa, Mus musculus
  >SEQUENCE_0656 TCR050 TRBV CDR3 3 Tcell_050 Mus_musculus
CASSDWGGYAEQFF

SEQ ID NO: 599 - protein, 15 aa, Mus musculus
  >SEQUENCE_0657 TCR051 TRBV CDR3 3 Tcell_051 Mus_musculus
CASSLTGYFSYEQYF

SEQ ID NO: 600 - protein, 13 aa, Mus musculus
  >SEQUENCE_0658 TCR052 TRBV CDR3 3 Tcell_052 Mus_musculus
CASSLRPNSDYTF

SEQ ID NO: 601 - protein, 14 aa, Mus musculus
  >SEQUENCE_0659 TCR053 TRBV CDR3 3 Tcell_053 Mus_musculus
CASGEGGNYAEQFF

SEQ ID NO: 602 - protein, 14 aa, Mus musculus
  >SEQUENCE_0660 TCR054 TRBV CDR3 3 Tcell_054 Mus_musculus
CASGEGGNYAEQFF

SEQ ID NO: 603 - protein, 14 aa, Mus musculus
  >SEQUENCE_0661 TCR055 TRBV CDR3 3 Tcell_055 Mus_musculus
CASSLTGDNERLFF

SEQ ID NO: 604 - protein, 15 aa, Mus musculus
  >SEQUENCE_0662 TCR056 TRBV CDR3 2 Tcell_056 Mus_musculus
CASSSRDTSNERLFF

SEQ ID NO: 605 - protein, 14 aa, Mus musculus
  >SEQUENCE_0663 TCR057 TRBV CDR3 2 Tcell_057 Mus_musculus
CASSDWGPQNTLYF

SEQ ID NO: 606 - protein, 14 aa, Mus musculus
  >SEQUENCE_0664 TCR058 TRBV CDR3 2 Tcell_058 Mus_musculus
CASGIGGANTEVFF

SEQ ID NO: 607 - protein, 15 aa, Mus musculus
  >SEQUENCE_0665 TCR059 TRBV CDR3 2 Tcell_059 Mus_musculus
CASRQTGGIYAEQFF

SEQ ID NO: 608 - protein, 14 aa, Mus musculus
  >SEQUENCE_0666 TCR060 TRBV CDR3 2 Tcell_060 Mus_musculus
CASSGTVSNERLFF

SEQ ID NO: 609 - protein, 16 aa, Mus musculus
  >SEQUENCE_0667 TCR061 TRBV CDR3 2 Tcell_061 Mus_musculus
CASSSWGGAGQNTLYF

SEQ ID NO: 610 - protein, 16 aa, Mus musculus
  >SEQUENCE_0668 TCR062 TRBV CDR3 2 Tcell_062 Mus_musculus
CASRRDWGADTGQLYF

SEQ ID NO: 611 - protein, 16 aa, Mus musculus
  >SEQUENCE_0669 TCR063 TRBV CDR3 2 Tcell_063 Mus_musculus
CASGDLGGRGQDTQYF

SEQ ID NO: 612 - protein, 12 aa, Mus musculus
  >SEQUENCE_0670 TCR064 TRBV CDR3 2 Tcell_064 Mus_musculus
CASGGSQNTLYF

SEQ ID NO: 613 - protein, 14 aa, Mus musculus
  >SEQUENCE_0671 TCR065 TRBV CDR3 2 Tcell_065 Mus_musculus
CASSKDISGNTLYF

SEQ ID NO: 614 - protein, 14 aa, Mus musculus
  >SEQUENCE_0672 TCR066 TRBV CDR3 2 Tcell_066 Mus_musculus
CASSKDISGNTLYF

SEQ ID NO: 615 - protein, 17 aa, Mus musculus
  >SEQUENCE_0673 TCR067 TRBV CDR3 2 Tcell_067 Mus_musculus
CASGDEQLGGSAETLYF

SEQ ID NO: 616 - protein, 14 aa, Mus musculus
  >SEQUENCE_0674 TCR068 TRBV CDR3 2 Tcell_068 Mus_musculus
CASSIGDIYAEQFF

SEQ ID NO: 617 - protein, 16 aa, Mus musculus
  >SEQUENCE_0675 TCR069 TRBV CDR3 2 Tcell_069 Mus_musculus
CASSRFQGASAETLYF

SEQ ID NO: 618 - protein, 14 aa, Mus musculus
  >SEQUENCE_0676 TCR070 TRBV CDR3 2 Tcell_070 Mus_musculus
CASSPRGGYAEQFF

SEQ ID NO: 619 - protein, 13 aa, Mus musculus
  >SEQUENCE_0677 TCR071 TRBV CDR3 2 Tcell_071 Mus_musculus
CASSYRVYAEQFF

SEQ ID NO: 620 - protein, 106 aa, Mus musculus
  >SEQUENCE_0678 TCR001 TRBV Full Length 17 Tcell_001 Mus_musculus
AQTIHQWPVAEIKAVGSPLSLGCTIKGKSSPNLYWYWQATGGTLQQLFYSITVGQVESVVQLNLSASRPKDDQFILSTEKLLLSHSGFYLCCAWSPQGTSNTEVFF

SEQ ID NO: 621 - protein, 104 aa, Mus musculus
  >SEQUENCE_0679 TCR002 TRBV Full Length 6 Tcell_002 Mus_musculus
SVLLYQKPNRDICQSGTSLKIQCVADSQVVSMFWYQQFQEQSLMLMATANEGSEATYESGFTKDKFPISRPNLTFSTLTVNNARPGDSSIYFCCSSIHLYEQNF

SEQ ID NO: 622 - protein, 103 aa, Mus musculus
  >SEQUENCE_0680 TCR003 TRBV Full Length 7 Tcell_003 Mus_musculus
NTKITQSPRYLILGRANKSLECEQHLGHNAMYWYKQSAEKPPELMFLYNLKQLIRNETVPSRFIPECPDSSKLLLHISAVDPEDSAVYFCCASSQGDWREQYF

SEQ ID NO: 623 - protein, 107 aa, Mus musculus
  >SEQUENCE_0681 TCR004 TRBV Full Length 4 Tcell_004 Mus_musculus
ETAVFQTPNYHVTQVGNEVSFNCKQTLGHDTMYWYKQDSKKLLKIMFSYNNKQLIVNETVPRRFSPQSSDKAHLNLRIKSVEPEDSAVYLCCASSELGGRGQNTLYF

SEQ ID NO: 624 - protein, 103 aa, Mus musculus
  >SEQUENCE_0682 TCR005 TRBV Full Length 6 Tcell_005 Mus_musculus
NAGVIQTPRHKVTGKGQEATLWCEPISGHSAVFWYRQTIVQGLEFLTYFRNQAPIDDSGMPKERFSAQMPNQSHSTLKIQSTQPQDSAVYLCCASSLTVEQYF

SEQ ID NO: 625 - protein, 106 aa, Mus musculus
  >SEQUENCE_0683 TCR006 TRBV Full Length 5 Tcell_006 Mus_musculus
DMKVTQMPRYLIKRMGENVLLECGQDMSHETMYWYRQDPGLGLQLIYISYDVDSNSEGDIPKGYRVSRKKREHFSLILDSAKTNQTSVYFCCASSPGQGANTEVFF

SEQ ID NO: 626 - protein, 105 aa, Mus musculus
  >SEQUENCE_0684 TCR007 TRBV Full Length 2 Tcell_007 Mus_musculus
VTLLEQNPRWRLVPRGQAVNLRCILKNSQYPWMSWYQQDLQKQLQWLFTLRSPGDKEVKSLPGADYLATRVTDTELRLQVANMSQGRTLYCCASSVGQSSYEQYF

SEQ ID NO: 627 - protein, 105 aa, Mus musculus
  >SEQUENCE_0685 TCR008 TRBV Full Length 3 Tcell_008 Mus_musculus
EAAVTQSPRSKVAVTGGKVTLSCHQTNNHDYMYWYRQDTGHGLRLIHYSYVADSTEKGDIPDGYKASRPSQENFSLILELASLSQTAVYFCCASSVGQSSYEQYF

SEQ ID NO: 628 - protein, 104 aa, Mus musculus
  >SEQUENCE_0686 TCR009 TRBV Full Length 2 Tcell_009 Mus_musculus
NTKITQSPRYLILGRANKSLECEQHLGHNARYWYKQSAEKPPELMFLYNLKQLIRNETVPSRFIPECPDSSKLLLHISAVDPEDSAVYFCCASSQDWQQDTQYF

SEQ ID NO: 629 - protein, 105 aa, Mus musculus
  >SEQUENCE_0687 TCR010 TRBV Full Length 2 Tcell_010 Mus_musculus
NTKITQSPRYLILGRANKSLECEQHLGHNAMYWYKQSAEKPPELMFLYNLKQLIRNETVPSRFIPECPDSSKLLLHISAVDPEDSAVYFCCASSQVPGQLYEQYF

SEQ ID NO: 630 - protein, 105 aa, Mus musculus
  >SEQUENCE_0688 TCR011 TRBV Full Length 2 Tcell_011 Mus_musculus
NAGVIQTPRHKVTGKGQEATLWCEPISGHSAVFWYRQTIVQGLEFLTYFRNQAPIDDSGMPKERFSAQMPNQSHSTLKIQSTQPQDSAVYLCCASSPWTVYEQYF

SEQ ID NO: 631 - protein, 108 aa, Mus musculus
  >SEQUENCE_0689 TCR012 TRBV Full Length 4 Tcell_012 Mus_musculus
EAAVTQSPRSKVAVTGGKVTLSCHQTNNHDYMYWYRQDTGHGLRLIHYSYVADSTEKGDIPDGYKASRPSQENFSLILELASLSQTAVYFCCASSDEDWGASAETLYF

SEQ ID NO: 632 - protein, 107 aa, Mus musculus
  >SEQUENCE_0690 TCR013 TRBV Full Length 2 Tcell_013 Mus_musculus
GPKVLQIPSHQIIDMGQMVTLNCDPVSNHLYFYWYKQILGQQMEFLVNFYNGKVMEKSKLFKDQFSVERPDGSYFTLKIQPTALEDSAVYFCCASSSGRRSGNTLYF

SEQ ID NO: 633 - protein, 108 aa, Mus musculus
  >SEQUENCE_0691 TCR014 TRBV Full Length 2 Tcell_014 Mus_musculus
GALVYQYPRRTICKSGTSMRMECQAVGFQATSVAWYRQSPQKTFELIALSTVNSAIKYEQNFTQEKFPISHPNLSFSSMTVLNAYLEDRGLYLCCGAFDRMSAETLYF

SEQ ID NO: 634 - protein, 107 aa, Mus musculus
  >SEQUENCE_0692 TCR015 TRBV Full Length 2 Tcell_015 Mus_musculus
GALVYQYPRRTICKSGTSMRMECQAVGFQATSVAWYRQSPQKTFELIALSTVNSAIKYEQNFTQEKFPISHPNLSFSSMTVLNAYLEDRGLYLCCGAREGTNSPLYF

SEQ ID NO: 635 - protein, 106 aa, Mus musculus
  >SEQUENCE_0693 TCR016 TRBV Full Length 2 Tcell_016 Mus_musculus
EAGVTQSPRYAVLQEGQAVSFWCDPISGHDTLYWYQQPRDQGPQLLVYFRDEAVIDNSQLPSDRFSAVRPKGTNSTLKIQSAKQGDTATYLCCASSFRLGGREQYF

SEQ ID NO: 636 - protein, 105 aa, Mus musculus
  >SEQUENCE_0694 TCR017 TRBV Full Length 5 Tcell_017 Mus_musculus
NSGVVQSPRYIIKGKGERSILKCIPISGHLSVAWYQQTQGQELKFFIQHYDKMERDKGNLPSRFSVQQFDDYHSEMNMSALELEDSAVYFCCASSLGGRSYEQYF

SEQ ID NO: 637 - protein, 105 aa, Mus musculus
  >SEQUENCE_0695 TCR018 TRBV Full Length 2 Tcell_018 Mus_musculus
EAAVTQSPRSKVAVTGGKVTLSCHQTNNHDYMYWYRQDTGHGLRLIHYSYVADSTEKGDIPDGYKASRPSQENFSLILELASLSQTAVYFCCASSDRGQAETLYF

SEQ ID NO: 638 - protein, 104 aa, Mus musculus
  >SEQUENCE_0696 TCR019 TRBV Full Length 2 Tcell_019 Mus_musculus
DAAVTQKPRYLIKMKGQEAEMKCIPEKGHTAVFWYQQKQSKELKFLIYFQNQQPLDQIDMVKERFSAVCPSSSLCSLGIRTCEAEDSALYLCCSSSLEQDTQYF

SEQ ID NO: 639 - protein, 104 aa, Mus musculus
  >SEQUENCE_0697 TCR020 TRBV Full Length 10 Tcell_020 Mus_musculus
EAAVTQSPRSKVAVTGGKVTLSCHQTNNHDYMYWYRQDTGHGLRLIHYSYVADSTEKGDIPDGYKASRPSQENFSLILELASLSQTAVYFCCASSNRGRDTQYF

SEQ ID NO: 640 - protein, 106 aa, Mus musculus
  >SEQUENCE_0698 TCR021 TRBV Full Length 3 Tcell_021 Mus_musculus
GGIITQTPKFLIGQEGQKLTLKCQQNFNHDTMYWYRQDSGKGLRLIYYSITENDLQKGDLSEGYDASREKKSSFSLTVTSAQKNEMAVFLCCASSPLDREDTEVFF

SEQ ID NO: 641 - protein, 106 aa, Mus musculus
  >SEQUENCE_0699 TCR022 TRBV Full Length 3 Tcell_022 Mus_musculus
EAGVTQSPRYAVLQEGQAVSFWCDPISGHDTLYWYQQPRDQGPQLLVYFRDEAVIDNSQLPSDRFSAVRPKGTNSTLKIQSAKQGDTATYLCCASSLGDSAETLYF

SEQ ID NO: 642 - protein, 105 aa, Mus musculus
  >SEQUENCE_0700 TCR023 TRBV Full Length 3 Tcell_023 Mus_musculus
EAAVTQSPRSKVAVTGGKVTLSCHQTNNHDYMYWYRQDTGHGLRLIHYSYVADSTEKGDIPDGYKASRPSQENFSLILELASLSQTAVYFCCASSDRASNTEVFF

SEQ ID NO: 643 - protein, 107 aa, Mus musculus
  >SEQUENCE_0701 TCR024 TRBV Full Length 3 Tcell_024 Mus_musculus
AQTIHQWPVAEIKAVGSPLSLGCTIKGKSSPNLYWYWQATGGTLQQLFYSITVGQVESVVQLNLSASRPKDDQFILSTEKLLLSHSGFYLCCAWSPPTGVSAETLYF

SEQ ID NO: 644 - protein, 105 aa, Mus musculus
  >SEQUENCE_0702 TCR025 TRBV Full Length 2 Tcell_025 Mus_musculus
AQTIHQWPVAEIKAVGSPLSLGCTIKGKSSPNLYWYWQATGGTLQQLFYSITVGQVESVVQLNLSASRPKDDQFILSTEKLLLSHSGFYLCCAWSPGLGGREQYF

SEQ ID NO: 645 - protein, 105 aa, Mus musculus
  >SEQUENCE_0703 TCR026 TRBV Full Length 2 Tcell_026 Mus_musculus
GGIITQTPKFLIGQEGQKLTLKCQQNFNHDTMYWYRQDSGKGLRLIYYSITENDLQKGDLSEGYDASREKKSSFSLTVTSAQKNEMAVFLCCASTQGATNERLFF

SEQ ID NO: 646 - protein, 103 aa, Mus musculus
  >SEQUENCE_0704 TCR027 TRBV Full Length 2 Tcell_027 Mus_musculus
DTTVKQNPRYKLARVGKPVNLICSQTMNHDTMYWYQKKPNQAPKLLLFYYDKILNREADTFEKFQSSRPNNSFCSLYIGSAGLEYSAMYLCCASSRNRDTLYF

SEQ ID NO: 647 - protein, 108 aa, Mus musculus
  >SEQUENCE_0705 TCR028 TRBV Full Length 2 Tcell_028 Mus_musculus
GALVYQYPRRTICKSGTSMRMECQAVGFQATSVAWYRQSPQKTFELIALSTVNSAIKYEQNFTQEKFPISHPNLSFSSMTVLNAYLEDRGLYLCCGASRGQTYAEQFF

SEQ ID NO: 648 - protein, 103 aa, Mus musculus
  >SEQUENCE_0706 TCR029 TRBV Full Length 2 Tcell_029 Mus_musculus
VTLLEQNPRWRLVPRGQAVNLRCILKNSQYPWMSWYQQDLQKQLQWLFTLRSPGDKEVKSLPGADYLATRVTDTELRLQVANMSQGRTLYCCSWDSSNERLFF

SEQ ID NO: 649 - protein, 104 aa, Mus musculus
  >SEQUENCE_0707 TCR030 TRBV Full Length 2 Tcell_030 Mus_musculus
SVLLYQKPNRDICQSGTSLKIQCVADSQVVSMFWYQQFQEQSLMLMATANEGSEATYESGFTKDKFPISRPNLTFSTLTVNNARPGDSSIYFCCSSIHLYEQYF

SEQ ID NO: 650 - protein, 104 aa, Mus musculus
  >SEQUENCE_0708 TCR031 TRBV Full Length 2 Tcell_031 Mus_musculus
EAAVTQSPRNKVAVTGGKVTLSCNQTNNHNNMYWYRQDTGHGLRLIHYSYGAGSTEKGDIPDGYKASRPSQENFSLILELATPSQTSVYFCCASGASGNQAPLF

SEQ ID NO: 651 - protein, 104 aa, Mus musculus
  >SEQUENCE_0709 TCR032 TRBV Full Length 2 Tcell_032 Mus_musculus
NSGVVQSPRYIIKGKGERSILKCIPISGHLSVAWYQQTQGQELKFFIQHYDKMERDKGNLPSRFSVQQFDDYHSEMNMSALELEDSAVYFCCASSLGTQDTQYF

SEQ ID NO: 652 - protein, 105 aa, Mus musculus
  >SEQUENCE_0710 TCR033 TRBV Full Length 2 Tcell_033 Mus_musculus
NTKITQSPRYLILGRANKSLECEQHLGHNAMYWYKQSAEKPPELMFLYNLKQLIRNETVPSRFIPECPDSSKLLLHISAVDPEDSAVYFCCASSQGLGVSYEQYF

SEQ ID NO: 653 - protein, 102 aa, Mus musculus
  >SEQUENCE_0711 TCR034 TRBV Full Length 2 Tcell_034 Mus_musculus
DPKIIQKPKYLVAVTGSEKILICEQYLGHNAMYWYRQSAKKPLEFMFSYSYQKLMDNQTASSRFQPQSSKKNHLDLQITALKPDDSATYFCCASSQESQVFF

SEQ ID NO: 654 - protein, 105 aa, Mus musculus
  >SEQUENCE_0712 TCR035 TRBV Full Length 2 Tcell_035 Mus_musculus
NSKVIQTPRYLVKGQGQKAKMRCIPEKGHPVVFWYQQNKNNEFKFLINFQNQEVLQQIDMTEKRFSAECPSNSPCSLEIQSSEAGDSALYLCCASSLTTNSDYTF

SEQ ID NO: 655 - protein, 105 aa, Mus musculus
  >SEQUENCE_0713 TCR036 TRBV Full Length 2 Tcell_036 Mus_musculus
VTLLEQNPRWRLVPRGQAVNLRCILKNSQYPWMSWYQQDLQKQLQWLFTLRSPGDKEVKSLPGADYLATRVTDTELRLQVANMSQGRTLYCCSADWGANTGQLYF

SEQ ID NO: 656 - protein, 107 aa, Mus musculus
  >SEQUENCE_0714 TCR037 TRBV Full Length 2 Tcell_037 Mus_musculus
EAAVTQSPRNKVAVTGGKVTLSCNQTNNHNNMYWYRQDTGHGLRLIHYSYGAGRTEKGDIPDGYKASRPSQENFSLILELATPSQTSVYFCCASGDALGGGQNTLYF

SEQ ID NO: 657 - protein, 106 aa, Mus musculus
  >SEQUENCE_0715 TCR038 TRBV Full Length 4 Tcell_038 Mus_musculus
EAGVTQSPRYAVLQEGQAVSFWCDPISGHDTLYWYQQPRDQGPQLLVYFRDEAVIDNSQLPSDRFSAVRPKGTNSTLKIQSAKQGDTATYLCCASSFLGRGDTQYF

SEQ ID NO: 658 - protein, 107 aa, Mus musculus
  >SEQUENCE_0716 TCR039 TRBV Full Length 12 Tcell_039 Mus_musculus
NAGVIQTPRHKVTGKGQEATLWCEPISGHSAVFWYRQTIVQGLEFLTYFRNQAPIDDSGMPKERFSAQMPNQSHSTLKIQSTQPQDSAVYLCCASSPDWGSSYEQYF

SEQ ID NO: 659 - protein, 104 aa, Mus musculus
  >SEQUENCE_0717 TCR040 TRBV Full Length 5 Tcell_040 Mus_musculus
GGIITQTPKFLIGQEGQKLTLKCQQNFNHDTMYWYRQDSGKGLRLIYYSITENDLQKGDLSEGYDASREKKSSFSLTVTSAQKNEMAVFLCCASRDREDERLFF

SEQ ID NO: 660 - protein, 105 aa, Mus musculus
  >SEQUENCE_0718 TCR041 TRBV Full Length 4 Tcell_041 Mus_musculus
NSGVVQSPRYIIKGKGERSILKCIPISGHLSVAWYQQTQGQELKFFIQHYDKMERDKGNLPSRFSVQQFDDYHSEMNMSALELEDSAVYFCCASSLDGSNERLFF

SEQ ID NO: 661 - protein, 106 aa, Mus musculus
  >SEQUENCE_0719 TCR042 TRBV Full Length 4 Tcell_042 Mus_musculus
GPKVLQIPSHQIIDMGQMVTLNCDPVSNHLYFYWYKQILGQQMEFLVNFYNGKVMEKSKLFKDQFSVERPDGSYFTLKIQPTALEDSAVYFCCASSPGQGNTEVFF

SEQ ID NO: 662 - protein, 108 aa, Mus musculus; identical to SEQ ID NO: 647
  >SEQUENCE_0720 TCR043 TRBV Full Length 3 Tcell_043 Mus_musculus

SEQ ID NO: 663 - protein, 104 aa, Mus musculus
  >SEQUENCE_0721 TCR044 TRBV Full Length 5 Tcell_044 Mus_musculus
EAAVTQSPRNKVTVTGGNVTLSCRQTNSHNYMYWYRQDTGHGLRLIHYSYGAGNLRIGDVPDGYKATRTTQEDFFLLLELASPSQTSLYFCCASSGTGVYEQYF

SEQ ID NO: 664 - protein, 104 aa, Mus musculus; identical to SEQ ID NO: 663
  >SEQUENCE_0722 TCR045 TRBV Full Length 3 Tcell_045 Mus_musculus

SEQ ID NO: 665 - protein, 105 aa, Mus musculus
  >SEQUENCE_0723 TCR046 TRBV Full Length 3 Tcell_046 Mus_musculus
EAAVTQSPRSKVAVTGGKVTLSCHQTNNHDYMYWYRQDTGHGLRLIHYSYVADSTEKGDIPDGYKASRPSQENFSLILELASLSQTAVYFCCASSALGGQDTQYF

SEQ ID NO: 666 - protein, 105 aa, Mus musculus
  >SEQUENCE_0724 TCR047 TRBV Full Length 3 Tcell_047 Mus_musculus
EAAVTQSPRNKVTVTGGNVTLSCRQTNSHNYMYWYRQDTGHGLRLIHYSYGAGNLRIGDVPDGYKATRTTQEDFFLLLELASPSQTSLYFCCASSLGGGQDTQYF

SEQ ID NO: 667 - protein, 105 aa, Mus musculus
  >SEQUENCE_0725 TCR048 TRBV Full Length 3 Tcell_048 Mus_musculus
NTKITQSPRYLILGRANKSLECEQHLGHNAMYWYKQSAEKPPELMFLYNLKQLIRNETVPSRFIPECPDSSKLLLHISAVDPEDSAVYFCCASSQVGAGNQAPLF

SEQ ID NO: 668 - protein, 106 aa, Mus musculus
  >SEQUENCE_0726 TCR049 TRBV Full Length 3 Tcell_049 Mus_musculus
GALVYQYPRRTICKSGTSMRMECQAVGFQATSVAWYRQSPQKTFELIALSTVNSAIKYEQNFTQEKFPISHPNLSFSSMTVLNAYLEDRGLYLCCGASTQNSDYTF

SEQ ID NO: 669 - protein, 105 aa, Mus musculus
  >SEQUENCE_0727 TCR050 TRBV Full Length 3 Tcell_050 Mus_musculus
EAAVTQSPRNKVTVTGGNVTLSCRQTNSHNYMYWYRQDTGHGLRLIHYSYGAGNLRIGDVPDGYKATRTTQEDFFLLLELASPSQTSLYFCCASSDWGGYAEQFF

SEQ ID NO: 670 - protein, 107 aa, Mus musculus
  >SEQUENCE_0728 TCR051 TRBV Full Length 3 Tcell_051 Mus_musculus
NSKVIQTPRYLVKGQGQKAKMRCIPEKGHPVVFWYQQNKNNEFKFLINFQNQEVLQQIDMTEKRFSAECPSNSPCSLEIQSSEAGDSALYLCCASSLTGYFSYEQYF

SEQ ID NO: 671 - protein, 105 aa, Mus musculus
  >SEQUENCE_0729 TCR052 TRBV Full Length 3 Tcell_052 Mus_musculus
NSKVIQTPRYLVKGQGQKAKMRCIPEKGHPVVFWYQQNKNNEFKFLINFQNQEVLQQIDMTEKRFSAECPSNSPCSLEIQSSEAGDSALYLCCASSLRPNSDYTF

SEQ ID NO: 672 - protein, 105 aa, Mus musculus
  >SEQUENCE_0730 TCR053 TRBV Full Length 3 Tcell_053 Mus_musculus
EAAVTQSPRNKVAVTGGKVTLSCNQTNNHNNMYWYRQDTGHGLRLIHYSYGAGSTEKGDIPDGYKASRPSQENFSLILELATPSQTSVYFCCASGEGGNYAEQFF

SEQ ID NO: 673 - protein, 105 aa, Mus musculus; identical to SEQ ID NO: 672
  >SEQUENCE_0731 TCR054 TRBV Full Length 3 Tcell_054 Mus_musculus

SEQ ID NO: 674 - protein, 105 aa, Mus musculus
  >SEQUENCE_0732 TCR055 TRBV Full Length 3 Tcell_055 Mus_musculus
NSGVVQSPRYIIKGKGERSILKCIPISGHLSVAWYQQTQGQELKFFIQHYDKMERDKGNLPSRFSVQQFDDYHSEMNMSALELEDSAVYFCCASSLTGDNERLFF

SEQ ID NO: 675 - protein, 107 aa, Mus musculus
  >SEQUENCE_0733 TCR056 TRBV Full Length 2 Tcell_056 Mus_musculus
GPKVLQIPSHQIIDMGQMVTLNCDPVSNHLYFYWYKQILGQQMEFLVNFYNGKVMEKSKLFKDQFSVERPDGSYFTLKIQPTALEDSAVYFCCASSSRDTSNERLFF

SEQ ID NO: 676 - protein, 105 aa, Mus musculus
  >SEQUENCE_0734 TCR057 TRBV Full Length 2 Tcell_057 Mus_musculus
EAAVTQSPRNKVTVTGGNVTLSCRQTNSHNYMYWYRQDTGHGLRLIHYSYGAGNLRIGDVPDGYKATRTTQEDFFLLLELASPSQTSLYFCCASSDWGPQNTLYF

SEQ ID NO: 677 - protein, 105 aa, Mus musculus
  >SEQUENCE_0735 TCR058 TRBV Full Length 2 Tcell_058 Mus_musculus
EAAVTQSPRNKVAVTGGKVTLSCNQTNNHNNMYWYRQDTGHGLRLIHYSYGAGSTEKGDIPDGYKASRPSQENFSLILELATPSQTSVYFCCASGIGGANTEVFF

SEQ ID NO: 678 - protein, 107 aa, Mus musculus
  >SEQUENCE_0736 TCR059 TRBV Full Length 2 Tcell_059 Mus_musculus
NSKVIQTPRYLVKGQGQKAKMRCIPEKGHPVVFWYQQNKNNEFKFLINFQNQEVLQQIDMTEKRFSAECPSNSPCSLEIQSSEAGDSALYLCCASRQTGGIYAEQFF

SEQ ID NO: 679 - protein, 104 aa, Mus musculus
  >SEQUENCE_0737 TCR060 TRBV Full Length 2 Tcell_060 Mus_musculus
NTKITQSPRYLILGRANKSLECEQHLGHNAMYWYKQSAEKPPELMFLYNLKQLIRNETVPSRFIPECPDSSKLLLHISAVDPEDSAVYFCCASSGTVSNERLFF

SEQ ID NO: 680 - protein, 107 aa, Mus musculus
  >SEQUENCE_0738 TCR061 TRBV Full Length 2 Tcell_061 Mus_musculus
ETAVFQTPNYHVTQVGNEVSFNCKQTLGHDTMYWYKQDSKKLLKIMFSYNNKQLIVNETVPRRFSPQSSDKAHLNLRIKSVEPEDSAVYLCCASSSWGGAGQNTLYF

SEQ ID NO: 681 - protein, 107 aa, Mus musculus
  >SEQUENCE_0739 TCR062 TRBV Full Length 2 Tcell_062 Mus_musculus
GGIITQTPKFLIGQEGQKLTLKCQQNFNHDTMYWYRQDSGKGLRLIYYSITENDLQKGDLSEGYDASREKKSSFSLTVTSAQKNEMAVFLCCASRRDWGADTGQLYF

SEQ ID NO: 682 - protein, 107 aa, Mus musculus
  >SEQUENCE_0740 TCR063 TRBV Full Length 2 Tcell_063 Mus_musculus
EAAVTQSPRNKVAVTGGKVTLSCNQTNNHNNMYWYRQDTGHGLRLIHYSYGAGSTEKGDIPDGYKASRPSQENFSLILELATPSQTSVYFCCASGDLGGRGQDTQYF

SEQ ID NO: 683 - protein, 103 aa, Mus musculus
  >SEQUENCE_0741 TCR064 TRBV Full Length 2 Tcell_064 Mus_musculus
EAAVTQSPRSKVAVTGGKVTLSCHQTNNHDYMYWYRQDTGHGLRLIHYSYVADSTEKGDIPDGYKASRPSQENFSLILELASLSQTAVYFCCASGGSQNTLYF

SEQ ID NO: 684 - protein, 105 aa, Mus musculus
  >SEQUENCE_0742 TCR065 TRBV Full Length 2 Tcell_065 Mus_musculus
GGIITQTPKFLIGQEGQKLTLKCQQNFNHDTMYWYRQDSGKGLRLIYYSITENDLQKGDLSEGYDASREKKSSFSLTVTSAQKNEMAVFLCCASSKDISGNTLYF

SEQ ID NO: 685 - protein, 105 aa, Mus musculus; identical to SEQ ID NO: 684
  >SEQUENCE_0743 TCR066 TRBV Full Length 2 Tcell_066 Mus_musculus

SEQ ID NO: 686 - protein, 108 aa, Mus musculus
  >SEQUENCE_0744 TCR067 TRBV Full Length 2 Tcell_067 Mus_musculus
EAAVTQSPRNKVAVTGGKVTLSCNQTNNHNNMYWYRQDTGHGLRLIHYSYGAGSTEKGDIPDGYKASRPSQENFSLILELATPSQTSVYFCCASGDEQLGGSAETLYF

SEQ ID NO: 687 - protein, 105 aa, Mus musculus
  >SEQUENCE_0745 TCR068 TRBV Full Length 2 Tcell_068 Mus_musculus
GGIITQTPKFLIGQEGQKLTLKCQQNFNHDTMYWYRQDSGKGLRLIYYSITENDLQKGDLSEGYDASREKKSSFSLTVTSAQKNEMAVFLCCASSIGDIYAEQFF

SEQ ID NO: 688 - protein, 107 aa, Mus musculus
  >SEQUENCE_0746 TCR069 TRBV Full Length 2 Tcell_069 Mus_musculus
DTTVKQNPRYKLARVGKPVNLICSQTMNHDTMYWYQKKPNQAPKLLLFYYDKILNREADTFEKFQSSRPNNSFCSLYIGSAGLEYSAMYLCCASSRFQGASAETLYF

SEQ ID NO: 689 - protein, 104 aa, Mus musculus
  >SEQUENCE_0747 TCR070 TRBV Full Length 2 Tcell_070 Mus_musculus
NTKITQSPRYLILGRANKSLECEQHLGHNAMYWYKQSAEKPPEVMFLYNLKQLIRNETVPSRFIPECPDSSKLLLHISAVDPEDSAVYFCCASSPRGGYAEQFF

SEQ ID NO: 690 - protein, 105 aa, Mus musculus
  >SEQUENCE_0748 TCR071 TRBV Full Length 2 Tcell_071 Mus_musculus
GPKVLQIPSHQIIDMGQMVTLNCDPVSNHLYFYWYKQILGQQMEFLVNFYNGKVMEKSKLFKDQFSVERPDGSYFTLKIQPTALEDSAVYFCCASSYRVYAEQFF

SEQ ID NO: 691 - protein, 6 aa, Mus musculus
  >SEQUENCE_0749 TCR001 TRAV CDR1 17 Tcell_001 Mus_musculus
DRNFQY

SEQ ID NO: 692 - protein, 6 aa, Mus musculus
  >SEQUENCE_0750 TCR002 TRAV CDR1 6 Tcell_002 Mus_musculus
YSATPY

SEQ ID NO: 693 - protein, 6 aa, Mus musculus
  >SEQUENCE_0751 TCR003 TRAV CDR1 7 Tcell_003 Mus_musculus
ASGYPA

SEQ ID NO: 694 - protein, 7 aa, Mus musculus
  >SEQUENCE_0752 TCR004 TRAV CDR1 4 Tcell_004 Mus_musculus
TISGNEY

SEQ ID NO: 695 - protein, 6 aa, Mus musculus
  >SEQUENCE_0753 TCR005 TRAV CDR1 6 Tcell_005 Mus_musculus
YSATPY

SEQ ID NO: 696 - protein, 6 aa, Mus musculus
  >SEQUENCE_0754 TCR006 TRAV CDR1 5 Tcell_006 Mus_musculus
DTASSY

SEQ ID NO: 697 - protein, 6 aa, Mus musculus
  >SEQUENCE_0755 TCR007 TRAV CDR1 2 Tcell_007 Mus_musculus
DGTSDN

SEQ ID NO: 698 - protein, 6 aa, Mus musculus
  >SEQUENCE_0756 TCR008 TRAV CDR1 3 Tcell_008 Mus_musculus
DSTFNY

SEQ ID NO: 699 - protein, 6 aa, Mus musculus
  >SEQUENCE_0757 TCR009 TRAV CDR1 2 Tcell_009 Mus_musculus
TAYSTF

SEQ ID NO: 700 - protein, 6 aa, Mus musculus
  >SEQUENCE_0758 TCR010 TRAV CDR1 2 Tcell_010 Mus_musculus
ASGYPA

SEQ ID NO: 701 - protein, 6 aa, Mus musculus
  >SEQUENCE_0759 TCR011 TRAV CDR1 2 Tcell_011 Mus_musculus
NDMFDY

SEQ ID NO: 702 - protein, 7 aa, Mus musculus
  >SEQUENCE_0760 TCR012 TRAV CDR1 4 Tcell_012 Mus_musculus
TISGNEY

SEQ ID NO: 703 - protein, 6 aa, Mus musculus
  >SEQUENCE_0761 TCR013 TRAV CDR1 2 Tcell_013 Mus_musculus
DGTSDN

SEQ ID NO: 704 - protein, 6 aa, Mus musculus
  >SEQUENCE_0762 TCR014 TRAV CDR1 2 Tcell_014 Mus_musculus
DSTFNY

SEQ ID NO: 705 - protein, 5 aa, Mus musculus
  >SEQUENCE_0763 TCR015 TRAV CDR1 2 Tcell_015 Mus_musculus
TSLNS

SEQ ID NO: 706 - protein, 5 aa, Mus musculus
  >SEQUENCE_0764 TCR016 TRAV CDR1 2 Tcell_016 Mus_musculus
TYTTV

SEQ ID NO: 707 - protein, 6 aa, Mus musculus
  >SEQUENCE_0765 TCR017 TRAV CDR1 5 Tcell_017 Mus_musculus
DPNSYY

SEQ ID NO: 708 - protein, 6 aa, Mus musculus
  >SEQUENCE_0766 TCR018 TRAV CDR1 2 Tcell_018 Mus_musculus
NSASNY

SEQ ID NO: 709 - protein, 6 aa, Mus musculus
  >SEQUENCE_0767 TCR019 TRAV CDR1 2 Tcell_019 Mus_musculus
DRNVDY

SEQ ID NO: 710 - protein, 6 aa, Mus musculus
  >SEQUENCE_0768 TCR020 TRAV CDR1 10 Tcell_020 Mus_musculus
DSATAY

SEQ ID NO: 711 - protein, 6 aa, Mus musculus
  >SEQUENCE_0769 TCR021 TRAV CDR1 3 Tcell_021 Mus_musculus
TSGFNG

SEQ ID NO: 712 - protein, 8 aa, Mus musculus
  >SEQUENCE_0770 TCR022 TRAV CDR1 3 Tcell_022 Mus_musculus
TTSIAYPN

SEQ ID NO: 713 - protein, 7 aa, Mus musculus
  >SEQUENCE_0771 TCR023 TRAV CDR1 3 Tcell_023 Mus_musculus
TQDSSYF

SEQ ID NO: 714 - protein, 6 aa, Mus musculus
  >SEQUENCE_0772 TCR024 TRAV CDR1 3 Tcell_024 Mus_musculus
ASGYPA

SEQ ID NO: 715 - protein, 7 aa, Mus musculus
  >SEQUENCE_0773 TCR025 TRAV CDR1 2 Tcell_025 Mus_musculus
TIYSNPF

SEQ ID NO: 716 - protein, 5 aa, Mus musculus
  >SEQUENCE_0774 TCR026 TRAV CDR1 2 Tcell_026 Mus_musculus
TTMRS

SEQ ID NO: 717 - protein, 6 aa, Mus musculus
  >SEQUENCE_0775 TCR027 TRAV CDR1 2 Tcell_027 Mus_musculus
TTTYPT

SEQ ID NO: 718 - protein, 6 aa, Mus musculus
  >SEQUENCE_0776 TCR028 TRAV CDR1 2 Tcell_028 Mus_musculus
TTTYPT

SEQ ID NO: 719 - protein, 5 aa, Mus musculus
  >SEQUENCE_0777 TCR029 TRAV CDR1 2 Tcell_029 Mus_musculus
TTMRA

SEQ ID NO: 720 - protein, 6 aa, Mus musculus
  >SEQUENCE_0778 TCR030 TRAV CDR1 2 Tcell_030 Mus_musculus
YSATPY

SEQ ID NO: 721 - protein, 6 aa, Mus musculus
  >SEQUENCE_0779 TCR031 TRAV CDR1 2 Tcell_031 Mus_musculus
NSASQS

SEQ ID NO: 722 - protein, 6 aa, Mus musculus
  >SEQUENCE_0780 TCR032 TRAV CDR1 2 Tcell_032 Mus_musculus
YSATPY

SEQ ID NO: 723 - protein, 6 aa, Mus musculus
  >SEQUENCE_0781 TCR033 TRAV CDR1 2 Tcell_033 Mus_musculus
TTGYPT

SEQ ID NO: 724 - protein, 6 aa, Mus musculus
  >SEQUENCE_0782 TCR034 TRAV CDR1 2 Tcell_034 Mus_musculus
ASGYPA

SEQ ID NO: 725 - protein, 7 aa, Mus musculus
  >SEQUENCE_0783 TCR035 TRAV CDR1 2 Tcell_035 Mus_musculus
TIYSNPY

SEQ ID NO: 726 - protein, 6 aa, Mus musculus
  >SEQUENCE_0784 TCR036 TRAV CDR1 2 Tcell_036 Mus_musculus
YFGTPY

SEQ ID NO: 727 - protein, 6 aa, Mus musculus
  >SEQUENCE_0785 TCR037 TRAV CDR1 2 Tcell_037 Mus_musculus
DSASVY

SEQ ID NO: 728 - protein, 7 aa, Mus musculus
  >SEQUENCE_0786 TCR038 TRAV CDR1 4 Tcell_038 Mus_musculus
TRDSSYF

SEQ ID NO: 729 - protein, 5 aa, Mus musculus
  >SEQUENCE_0787 TCR039 TRAV CDR1 12 Tcell_039 Mus_musculus
STATQ

SEQ ID NO: 730 - protein, 6 aa, Mus musculus
  >SEQUENCE_0788 TCR040 TRAV CDR1 5 Tcell_040 Mus_musculus
TAYSTF

SEQ ID NO: 731 - protein, 7 aa, Mus musculus
  >SEQUENCE_0789 TCR041 TRAV CDR1 4 Tcell_041 Mus_musculus
TQDSSYF

SEQ ID NO: 732 - protein, 6 aa, Mus musculus
  >SEQUENCE_0790 TCR042 TRAV CDR1 4 Tcell_042 Mus_musculus
TAYSTF

SEQ ID NO: 733 - protein, 6 aa, Mus musculus
  >SEQUENCE_0791 TCR043 TRAV CDR1 3 Tcell_043 Mus_musculus
TTTYPT

SEQ ID NO: 734 - protein, 6 aa, Mus musculus
  >SEQUENCE_0792 TCR044 TRAV CDR1 5 Tcell_044 Mus_musculus
STYSPF

SEQ ID NO: 735 - protein, 6 aa, Mus musculus
  >SEQUENCE_0793 TCR045 TRAV CDR1 3 Tcell_045 Mus_musculus
ASGYPA

SEQ ID NO: 736 - protein, 7 aa, Mus musculus
  >SEQUENCE_0794 TCR046 TRAV CDR1 3 Tcell_046 Mus_musculus
TAYSDVA

SEQ ID NO: 737 - protein, 6 aa, Mus musculus
  >SEQUENCE_0795 TCR047 TRAV CDR1 3 Tcell_047 Mus_musculus
YSGTPY

SEQ ID NO: 738 - protein, 6 aa, Mus musculus
  >SEQUENCE_0796 TCR048 TRAV CDR1 3 Tcell_048 Mus_musculus
NSAFDY

SEQ ID NO: 739 - protein, 8 aa, Mus musculus
  >SEQUENCE_0797 TCR049 TRAV CDR1 3 Tcell_049 Mus_musculus
TTSIAYPN

SEQ ID NO: 740 - protein, 6 aa, Mus musculus
  >SEQUENCE_0798 TCR050 TRAV CDR1 3 Tcell_050 Mus_musculus
DRNFQY

SEQ ID NO: 741 - protein, 6 aa, Mus musculus
  >SEQUENCE_0799 TCR051 TRAV CDR1 3 Tcell_051 Mus_musculus
DRNFQY

SEQ ID NO: 742 - protein, 6 aa, Mus musculus
  >SEQUENCE_0800 TCR052 TRAV CDR1 3 Tcell_052 Mus_musculus
DPNSYY

SEQ ID NO: 743 - protein, 5 aa, Mus musculus
  >SEQUENCE_0801 TCR053 TRAV CDR1 3 Tcell_053 Mus_musculus
STATR

SEQ ID NO: 744 - protein, 6 aa, Mus musculus
  >SEQUENCE_0802 TCR054 TRAV CDR1 3 Tcell_054 Mus_musculus
DRNVDY

SEQ ID NO: 745 - protein, 6 aa, Mus musculus
  >SEQUENCE_0803 TCR055 TRAV CDR1 3 Tcell_055 Mus_musculus
YSATPY

SEQ ID NO: 746 - protein, 6 aa, Mus musculus
  >SEQUENCE_0804 TCR056 TRAV CDR1 2 Tcell_056 Mus_musculus
DSTFNY

SEQ ID NO: 747 - protein, 6 aa, Mus musculus
  >SEQUENCE_0805 TCR057 TRAV CDR1 2 Tcell_057 Mus_musculus
DRNVDY

SEQ ID NO: 748 - protein, 6 aa, Mus musculus
  >SEQUENCE_0806 TCR058 TRAV CDR1 2 Tcell_058 Mus_musculus
DPNSYY

SEQ ID NO: 749 - protein, 6 aa, Mus musculus
  >SEQUENCE_0807 TCR059 TRAV CDR1 2 Tcell_059 Mus_musculus
NSAFDY

SEQ ID NO: 750 - protein, 5 aa, Mus musculus
  >SEQUENCE_0808 TCR060 TRAV CDR1 2 Tcell_060 Mus_musculus
TTMRA

SEQ ID NO: 751 - protein, 6 aa, Mus musculus
  >SEQUENCE_0809 TCR061 TRAV CDR1 2 Tcell_061 Mus_musculus
YFGTPY

SEQ ID NO: 752 - protein, 7 aa, Mus musculus
  >SEQUENCE_0810 TCR062 TRAV CDR1 2 Tcell_062 Mus_musculus
NIATSEY

SEQ ID NO: 753 - protein, 8 aa, Mus musculus
  >SEQUENCE_0811 TCR063 TRAV CDR1 2 Tcell_063 Mus_musculus
ATSIGYPN

SEQ ID NO: 754 - protein, 6 aa, Mus musculus
  >SEQUENCE_0812 TCR064 TRAV CDR1 2 Tcell_064 Mus_musculus
DPNSYY

SEQ ID NO: 755 - protein, 6 aa, Mus musculus
  >SEQUENCE_0813 TCR065 TRAV CDR1 2 Tcell_065 Mus_musculus
DTASSY

SEQ ID NO: 756 - protein, 6 aa, Mus musculus
  >SEQUENCE_0814 TCR066 TRAV CDR1 2 Tcell_066 Mus_musculus
ASGYPA

SEQ ID NO: 757 - protein, 7 aa, Mus musculus
  >SEQUENCE_0815 TCR067 TRAV CDR1 2 Tcell_067 Mus_musculus
TQDSSYF

SEQ ID NO: 758 - protein, 8 aa, Mus musculus
  >SEQUENCE_0816 TCR068 TRAV CDR1 2 Tcell_068 Mus_musculus
TTSIAYPN

SEQ ID NO: 759 - protein, 5 aa, Mus musculus
  >SEQUENCE_0817 TCR069 TRAV CDR1 2 Tcell_069 Mus_musculus
IAATT

SEQ ID NO: 760 - protein, 5 aa, Mus musculus
  >SEQUENCE_0818 TCR070 TRAV CDR1 2 Tcell_070 Mus_musculus
TYTTV

SEQ ID NO: 761 - protein, 6 aa, Mus musculus
  >SEQUENCE_0819 TCR071 TRAV CDR1 2 Tcell_071 Mus_musculus
NSASNY

SEQ ID NO: 762 - protein, 6 aa, Mus musculus
  >SEQUENCE_0820 TCR001 TRAV CDR2 17 Tcell_001 Mus_musculus
IFSDGD

SEQ ID NO: 763 - protein, 8 aa, Mus musculus
  >SEQUENCE_0821 TCR002 TRAV CDR2 6 Tcell_002 Mus_musculus
YYSGDPVV

SEQ ID NO: 764 - protein, 7 aa, Mus musculus
  >SEQUENCE_0822 TCR003 TRAV CDR2 7 Tcell_003 Mus_musculus
ASRDKEK

SEQ ID NO: 765 - protein, 5 aa, Mus musculus
  >SEQUENCE_0823 TCR004 TRAV CDR2 4 Tcell_004 Mus_musculus
GLQQN

SEQ ID NO: 766 - protein, 8 aa, Mus musculus
  >SEQUENCE_0824 TCR005 TRAV CDR2 6 Tcell_005 Mus_musculus
YYSGDPVV

SEQ ID NO: 767 - protein, 7 aa, Mus musculus
  >SEQUENCE_0825 TCR006 TRAV CDR2 5 Tcell_006 Mus_musculus
IRSNVDR

SEQ ID NO: 768 - protein, 6 aa, Mus musculus
  >SEQUENCE_0826 TCR007 TRAV CDR2 2 Tcell_007 Mus_musculus
IFSDGE

SEQ ID NO: 769 - protein, 7 aa, Mus musculus
  >SEQUENCE_0827 TCR008 TRAV CDR2 3 Tcell_008 Mus_musculus
IRSVSDK

SEQ ID NO: 770 - protein, 7 aa, Mus musculus
  >SEQUENCE_0828 TCR009 TRAV CDR2 2 Tcell_009 Mus_musculus
SSTDNKR

SEQ ID NO: 771 - protein, 7 aa, Mus musculus
  >SEQUENCE_0829 TCR010 TRAV CDR2 2 Tcell_010 Mus_musculus
ASRDKEK

SEQ ID NO: 772 - protein, 7 aa, Mus musculus
  >SEQUENCE_0830 TCR011 TRAV CDR2 2 Tcell_011 Mus_musculus
VRSNVDK

SEQ ID NO: 773 - protein, 5 aa, Mus musculus
  >SEQUENCE_0831 TCR012 TRAV CDR2 4 Tcell_012 Mus_musculus
GLQQN

SEQ ID NO: 774 - protein, 6 aa, Mus musculus
  >SEQUENCE_0832 TCR013 TRAV CDR2 2 Tcell_013 Mus_musculus
IFSDGE

SEQ ID NO: 775 - protein, 7 aa, Mus musculus
  >SEQUENCE_0833 TCR014 TRAV CDR2 2 Tcell_014 Mus_musculus
IRSVSDK

SEQ ID NO: 776 - protein, 5 aa, Mus musculus
  >SEQUENCE_0834 TCR015 TRAV CDR2 2 Tcell_015 Mus_musculus
NPSGT

SEQ ID NO: 777 - protein, 7 aa, Mus musculus
  >SEQUENCE_0835 TCR016 TRAV CDR2 2 Tcell_016 Mus_musculus
IRSNERE

SEQ ID NO: 778 - protein, 7 aa, Mus musculus
  >SEQUENCE_0836 TCR017 TRAV CDR2 5 Tcell_017 Mus_musculus
VFSSTEI

SEQ ID NO: 779 - protein, 7 aa, Mus musculus
  >SEQUENCE_0837 TCR018 TRAV CDR2 2 Tcell_018 Mus_musculus
IRSNMER

SEQ ID NO: 780 - protein, 6 aa, Mus musculus
  >SEQUENCE_0838 TCR019 TRAV CDR2 2 Tcell_019 Mus_musculus
IFSNGE

SEQ ID NO: 781 - protein, 7 aa, Mus musculus
  >SEQUENCE_0839 TCR020 TRAV CDR2 10 Tcell_020 Mus_musculus
VFSNVDR

SEQ ID NO: 782 - protein, 6 aa, Mus musculus
  >SEQUENCE_0840 TCR021 TRAV CDR2 3 Tcell_021 Mus_musculus
VVLDGL

SEQ ID NO: 783 - protein, 7 aa, Mus musculus
  >SEQUENCE_0841 TCR022 TRAV CDR2 3 Tcell_022 Mus_musculus
VITAGQK

SEQ ID NO: 784 - protein, 8 aa, Mus musculus
  >SEQUENCE_0842 TCR023 TRAV CDR2 3 Tcell_023 Mus_musculus
QDSYKKEN

SEQ ID NO: 785 - protein, 7 aa, Mus musculus
  >SEQUENCE_0843 TCR024 TRAV CDR2 3 Tcell_024 Mus_musculus
ASRDKEK

SEQ ID NO: 786 - protein, 7 aa, Mus musculus
  >SEQUENCE_0844 TCR025 TRAV CDR2 2 Tcell_025 Mus_musculus
SSTDNKR

SEQ ID NO: 787 - protein, 5 aa, Mus musculus
  >SEQUENCE_0845 TCR026 TRAV CDR2 2 Tcell_026 Mus_musculus
LASGT

SEQ ID NO: 788 - protein, 7 aa, Mus musculus
  >SEQUENCE_0846 TCR027 TRAV CDR2 2 Tcell_027 Mus_musculus
VTTANNK

SEQ ID NO: 789 - protein, 7 aa, Mus musculus
  >SEQUENCE_0847 TCR028 TRAV CDR2 2 Tcell_028 Mus_musculus
VTTANNK

SEQ ID NO: 790 - protein, 5 aa, Mus musculus
  >SEQUENCE_0848 TCR029 TRAV CDR2 2 Tcell_029 Mus_musculus
LASGT

SEQ ID NO: 791 - protein, 8 aa, Mus musculus
  >SEQUENCE_0849 TCR030 TRAV CDR2 2 Tcell_030 Mus_musculus
YYSGDPVV

SEQ ID NO: 792 - protein, 6 aa, Mus musculus
  >SEQUENCE_0850 TCR031 TRAV CDR2 2 Tcell_031 Mus_musculus
IFSNGN

SEQ ID NO: 793 - protein, 8 aa, Mus musculus
  >SEQUENCE_0851 TCR032 TRAV CDR2 2 Tcell_032 Mus_musculus
YYSGDPVV

SEQ ID NO: 794 - protein, 7 aa, Mus musculus
  >SEQUENCE_0852 TCR033 TRAV CDR2 2 Tcell_033 Mus_musculus
VTTANNK

SEQ ID NO: 795 - protein, 7 aa, Mus musculus
  >SEQUENCE_0853 TCR034 TRAV CDR2 2 Tcell_034 Mus_musculus
ASRDKEK

SEQ ID NO: 796 - protein, 7 aa, Mus musculus
  >SEQUENCE_0854 TCR035 TRAV CDR2 2 Tcell_035 Mus_musculus
NFTDNKR

SEQ ID NO: 797 - protein, 8 aa, Mus musculus
  >SEQUENCE_0855 TCR036 TRAV CDR2 2 Tcell_036 Mus_musculus
YYPGDPVV

SEQ ID NO: 798 - protein, 7 aa, Mus musculus
  >SEQUENCE_0856 TCR037 TRAV CDR2 2 Tcell_037 Mus_musculus
IRSNMER

SEQ ID NO: 799 - protein, 8 aa, Mus musculus
  >SEQUENCE_0857 TCR038 TRAV CDR2 4 Tcell_038 Mus_musculus
QDSYKKEN

SEQ ID NO: 800 - protein, 5 aa, Mus musculus
  >SEQUENCE_0858 TCR039 TRAV CDR2 12 Tcell_039 Mus_musculus
NPSGT

SEQ ID NO: 801 - protein, 7 aa, Mus musculus
  >SEQUENCE_0859 TCR040 TRAV CDR2 5 Tcell_040 Mus_musculus
SSTDNKR

SEQ ID NO: 802 - protein, 8 aa, Mus musculus
  >SEQUENCE_0860 TCR041 TRAV CDR2 4 Tcell_041 Mus_musculus
QDSYKKEN

SEQ ID NO: 803 - protein, 7 aa, Mus musculus
  >SEQUENCE_0861 TCR042 TRAV CDR2 4 Tcell_042 Mus_musculus
SSTDNKR

SEQ ID NO: 804 - protein, 7 aa, Mus musculus
  >SEQUENCE_0862 TCR043 TRAV CDR2 3 Tcell_043 Mus_musculus
VTTANNK

SEQ ID NO: 805 - protein, 7 aa, Mus musculus
  >SEQUENCE_0863 TCR044 TRAV CDR2 5 Tcell_044 Mus_musculus
SFTDNKR

SEQ ID NO: 806 - protein, 7 aa, Mus musculus
  >SEQUENCE_0864 TCR045 TRAV CDR2 3 Tcell_045 Mus_musculus
ASRDKEK

SEQ ID NO: 807 - protein, 7 aa, Mus musculus
  >SEQUENCE_0865 TCR046 TRAV CDR2 3 Tcell_046 Mus_musculus
SSTDNKR

SEQ ID NO: 808 - protein, 8 aa, Mus musculus
  >SEQUENCE_0866 TCR047 TRAV CDR2 3 Tcell_047 Mus_musculus
YYSGDPVV

SEQ ID NO: 809 - protein, 7 aa, Mus musculus
  >SEQUENCE_0867 TCR048 TRAV CDR2 3 Tcell_048 Mus_musculus
IRSVSDK

SEQ ID NO: 810 - protein, 7 aa, Mus musculus
  >SEQUENCE_0868 TCR049 TRAV CDR2 3 Tcell_049 Mus_musculus
VITAGQK

SEQ ID NO: 811 - protein, 6 aa, Mus musculus
  >SEQUENCE_0869 TCR050 TRAV CDR2 3 Tcell_050 Mus_musculus
IFSDGD

SEQ ID NO: 812 - protein, 6 aa, Mus musculus
  >SEQUENCE_0870 TCR051 TRAV CDR2 3 Tcell_051 Mus_musculus
IFSDGD

SEQ ID NO: 813 - protein, 7 aa, Mus musculus
  >SEQUENCE_0871 TCR052 TRAV CDR2 3 Tcell_052 Mus_musculus
VFSSTEI

SEQ ID NO: 814 - protein, 5 aa, Mus musculus
  >SEQUENCE_0872 TCR053 TRAV CDR2 3 Tcell_053 Mus_musculus
NPSGT

SEQ ID NO: 815 - protein, 6 aa, Mus musculus
  >SEQUENCE_0873 TCR054 TRAV CDR2 3 Tcell_054 Mus_musculus
IFSNGE

SEQ ID NO: 816 - protein, 8 aa, Mus musculus
  >SEQUENCE_0874 TCR055 TRAV CDR2 3 Tcell_055 Mus_musculus
YYSGDPVV

SEQ ID NO: 817 - protein, 7 aa, Mus musculus
  >SEQUENCE_0875 TCR056 TRAV CDR2 2 Tcell_056 Mus_musculus
IRSVSDK

SEQ ID NO: 818 - protein, 6 aa, Mus musculus
  >SEQUENCE_0876 TCR057 TRAV CDR2 2 Tcell_057 Mus_musculus
IFSNGE

SEQ ID NO: 819 - protein, 7 aa, Mus musculus
  >SEQUENCE_0877 TCR058 TRAV CDR2 2 Tcell_058 Mus_musculus
VFSSTEI

SEQ ID NO: 820 - protein, 7 aa, Mus musculus
  >SEQUENCE_0878 TCR059 TRAV CDR2 2 Tcell_059 Mus_musculus
IRSVSDK

SEQ ID NO: 821 - protein, 5 aa, Mus musculus
  >SEQUENCE_0879 TCR060 TRAV CDR2 2 Tcell_060 Mus_musculus
LASGT

SEQ ID NO: 822 - protein, 8 aa, Mus musculus
  >SEQUENCE_0880 TCR061 TRAV CDR2 2 Tcell_061 Mus_musculus
YYPGDPVV

SEQ ID NO: 823 - protein, 5 aa, Mus musculus
  >SEQUENCE_0881 TCR062 TRAV CDR2 2 Tcell_062 Mus_musculus
GYKDY

SEQ ID NO: 824 - protein, 7 aa, Mus musculus
  >SEQUENCE_0882 TCR063 TRAV CDR2 2 Tcell_063 Mus_musculus
VITAGQK

SEQ ID NO: 825 - protein, 7 aa, Mus musculus
  >SEQUENCE_0883 TCR064 TRAV CDR2 2 Tcell_064 Mus_musculus
VFSSTEI

SEQ ID NO: 826 - protein, 7 aa, Mus musculus
  >SEQUENCE_0884 TCR065 TRAV CDR2 2 Tcell_065 Mus_musculus
IRSNVDR

SEQ ID NO: 827 - protein, 7 aa, Mus musculus
  >SEQUENCE_0885 TCR066 TRAV CDR2 2 Tcell_066 Mus_musculus
ASRDKEK

SEQ ID NO: 828 - protein, 8 aa, Mus musculus
  >SEQUENCE_0886 TCR067 TRAV CDR2 2 Tcell_067 Mus_musculus
QDSYKKEN

SEQ ID NO: 829 - protein, 7 aa, Mus musculus
  >SEQUENCE_0887 TCR068 TRAV CDR2 2 Tcell_068 Mus_musculus
VITAGQK

SEQ ID NO: 830 - protein, 5 aa, Mus musculus
  >SEQUENCE_0888 TCR069 TRAV CDR2 2 Tcell_069 Mus_musculus
LVPGT

SEQ ID NO: 831 - protein, 7 aa, Mus musculus
  >SEQUENCE_0889 TCR070 TRAV CDR2 2 Tcell_070 Mus_musculus
IRSNERE

SEQ ID NO: 832 - protein, 7 aa, Mus musculus
  >SEQUENCE_0890 TCR071 TRAV CDR2 2 Tcell_071 Mus_musculus
IRSNMER

SEQ ID NO: 833 - protein, 12 aa, Mus musculus
  >SEQUENCE_0891 TCR001 TRAV CDR3 17 Tcell_001 Mus_musculus
CAAPSNYNVLYF

SEQ ID NO: 834 - protein, 13 aa, Mus musculus
  >SEQUENCE_0892 TCR002 TRAV CDR3 6 Tcell_002 Mus_musculus
CAVRNTGYQNFYF

SEQ ID NO: 835 - protein, 14 aa, Mus musculus
  >SEQUENCE_0893 TCR003 TRAV CDR3 7 Tcell_003 Mus_musculus
CALGDHNYAQGLTF

SEQ ID NO: 836 - protein, 13 aa, Mus musculus
  >SEQUENCE_0894 TCR004 TRAV CDR3 4 Tcell_004 Mus_musculus
CILRGSALGRLHF

SEQ ID NO: 837 - protein, 14 aa, Mus musculus
  >SEQUENCE_0895 TCR005 TRAV CDR3 6 Tcell_005 Mus_musculus
CAVSASTGNYKYVF

SEQ ID NO: 838 - protein, 14 aa, Mus musculus
  >SEQUENCE_0896 TCR006 TRAV CDR3 5 Tcell_006 Mus_musculus
CAASRSSSFSKLVF

SEQ ID NO: 839 - protein, 15 aa, Mus musculus
  >SEQUENCE_0897 TCR007 TRAV CDR3 2 Tcell_007 Mus_musculus
CAVSMRAGNTGKLIF

SEQ ID NO: 840 - protein, 15 aa, Mus musculus
  >SEQUENCE_0898 TCR008 TRAV CDR3 3 Tcell_008 Mus_musculus
CAASGYGSSGNKLIF

SEQ ID NO: 841 - protein, 18 aa, Mus musculus
  >SEQUENCE_0899 TCR009 TRAV CDR3 2 Tcell_009 Mus_musculus
CALSDREPQQGTGSKLSF

SEQ ID NO: 842 - protein, 15 aa, Mus musculus
  >SEQUENCE_0900 TCR010 TRAV CDR3 2 Tcell_010 Mus_musculus
CALGDKSTNTGKLTF

SEQ ID NO: 843 - protein, 12 aa, Mus musculus
  >SEQUENCE_0901 TCR011 TRAV CDR3 2 Tcell_011 Mus_musculus
CAATGGNNKLTF

SEQ ID NO: 844 - protein, 15 aa, Mus musculus
  >SEQUENCE_0902 TCR012 TRAV CDR3 4 Tcell_012 Mus_musculus
CILRANQGGSAKLIF

SEQ ID NO: 845 - protein, 12 aa, Mus musculus
  >SEQUENCE_0903 TCR013 TRAV CDR3 2 Tcell_013 Mus_musculus
CAVGGTGSKLSF

SEQ ID NO: 846 - protein, 13 aa, Mus musculus
  >SEQUENCE_0904 TCR014 TRAV CDR3 2 Tcell_014 Mus_musculus
CAARGTGNYKYVF

SEQ ID NO: 847 - protein, 12 aa, Mus musculus
  >SEQUENCE_0905 TCR015 TRAV CDR3 2 Tcell_015 Mus_musculus
CAWLTGNYKYVF

SEQ ID NO: 848 - protein, 14 aa, Mus musculus
  >SEQUENCE_0906 TCR016 TRAV CDR3 2 Tcell_016 Mus_musculus
CATDRGSALGRLHF

SEQ ID NO: 849 - protein, 13 aa, Mus musculus
  >SEQUENCE_0907 TCR017 TRAV CDR3 5 Tcell_017 Mus_musculus
CAVKDTNTGKLTF

SEQ ID NO: 850 - protein, 14 aa, Mus musculus
  >SEQUENCE_0908 TCR018 TRAV CDR3 2 Tcell_018 Mus_musculus
CAASARGTGSKLSF

SEQ ID NO: 851 - protein, 13 aa, Mus musculus
  >SEQUENCE_0909 TCR019 TRAV CDR3 2 Tcell_019 Mus_musculus
CAALSGSFNKLTF

SEQ ID NO: 852 - protein, 15 aa, Mus musculus
  >SEQUENCE_0910 TCR020 TRAV CDR3 10 Tcell_020 Mus_musculus
CAVSQATGGNNKLTF

SEQ ID NO: 853 - protein, 12 aa, Mus musculus
  >SEQUENCE_0911 TCR021 TRAV CDR3 3 Tcell_021 Mus_musculus
CAVMDSNYQLIW

SEQ ID NO: 854 - protein, 15 aa, Mus musculus
  >SEQUENCE_0912 TCR022 TRAV CDR3 3 Tcell_022 Mus_musculus
CALGAGSSNTNKVVF

SEQ ID NO: 855 - protein, 8 aa, Mus musculus
  >SEQUENCE_0913 TCR023 TRAV CDR3 3 Tcell_023 Mus_musculus
CAMREGAT

SEQ ID NO: 856 - protein, 14 aa, Mus musculus
  >SEQUENCE_0914 TCR024 TRAV CDR3 3 Tcell_024 Mus_musculus
CALRANTGYQNFYF

SEQ ID NO: 857 - protein, 12 aa, Mus musculus
  >SEQUENCE_0915 TCR025 TRAV CDR3 2 Tcell_025 Mus_musculus
CALRNYAQGLTF

SEQ ID NO: 858 - protein, 14 aa, Mus musculus
  >SEQUENCE_0916 TCR026 TRAV CDR3 2 Tcell_026 Mus_musculus
CAAEASSGSWQLIF

SEQ ID NO: 859 - protein, 13 aa, Mus musculus
  >SEQUENCE_0917 TCR027 TRAV CDR3 2 Tcell_027 Mus_musculus
CALVRYNQGKLIF

SEQ ID NO: 860 - protein, 13 aa, Mus musculus
  >SEQUENCE_0918 TCR028 TRAV CDR3 2 Tcell_028 Mus_musculus
CALVDWSNNRIFF

SEQ ID NO: 861 - protein, 15 aa, Mus musculus
  >SEQUENCE_0919 TCR029 TRAV CDR3 2 Tcell_029 Mus_musculus
CAAVDRGSALGRLHF

SEQ ID NO: 862 - protein, 13 aa, Mus musculus
  >SEQUENCE_0920 TCR030 TRAV CDR3 2 Tcell_030 Mus_musculus
CAVRNTGYQNFYF

SEQ ID NO: 863 - protein, 13 aa, Mus musculus
  >SEQUENCE_0921 TCR031 TRAV CDR3 2 Tcell_031 Mus_musculus
CAVSGNSNNRIFF

SEQ ID NO: 864 - protein, 14 aa, Mus musculus
  >SEQUENCE_0922 TCR032 TRAV CDR3 2 Tcell_032 Mus_musculus
CAVSARNAGAKLTF

SEQ ID NO: 865 - protein, 13 aa, Mus musculus
  >SEQUENCE_0923 TCR033 TRAV CDR3 2 Tcell_033 Mus_musculus
CAMSDLAGNKLTF

SEQ ID NO: 866 - protein, 14 aa, Mus musculus
  >SEQUENCE_0924 TCR034 TRAV CDR3 2 Tcell_034 Mus_musculus
CALANMDSNYQLIW

SEQ ID NO: 867 - protein, 13 aa, Mus musculus
  >SEQUENCE_0925 TCR035 TRAV CDR3 2 Tcell_035 Mus_musculus
CALASGGNYKPTF

SEQ ID NO: 868 - protein, 16 aa, Mus musculus
  >SEQUENCE_0926 TCR036 TRAV CDR3 2 Tcell_036 Mus_musculus
CAVSPMTNSAGNKLTF

SEQ ID NO: 869 - protein, 12 aa, Mus musculus
  >SEQUENCE_0927 TCR037 TRAV CDR3 2 Tcell_037 Mus_musculus
CSATTGGYKVVF

SEQ ID NO: 870 - protein, 14 aa, Mus musculus
  >SEQUENCE_0928 TCR038 TRAV CDR3 4 Tcell_038 Mus_musculus
CAMREGNNNNAPRF

SEQ ID NO: 871 - protein, 16 aa, Mus musculus
  >SEQUENCE_0929 TCR039 TRAV CDR3 12 Tcell_039 Mus_musculus
CATLIDRGSALGRLHF

SEQ ID NO: 872 - protein, 13 aa, Mus musculus
  >SEQUENCE_0930 TCR040 TRAV CDR3 5 Tcell_040 Mus_musculus
CAATTASLGKLQF

SEQ ID NO: 873 - protein, 17 aa, Mus musculus
  >SEQUENCE_0931 TCR041 TRAV CDR3 4 Tcell_041 Mus_musculus
CAMREALSGGSNAKLTF

SEQ ID NO: 874 - protein, 12 aa, Mus musculus
  >SEQUENCE_0932 TCR042 TRAV CDR3 4 Tcell_042 Mus_musculus
CAQTSSGQKLVF

SEQ ID NO: 875 - protein, 13 aa, Mus musculus
  >SEQUENCE_0933 TCR043 TRAV CDR3 3 Tcell_043 Mus_musculus
CALVDWSNNRIFF

SEQ ID NO: 876 - protein, 14 aa, Mus musculus
  >SEQUENCE_0934 TCR044 TRAV CDR3 5 Tcell_044 Mus_musculus
CALRASSGSWQLIF

SEQ ID NO: 877 - protein, 9 aa, Mus musculus
  >SEQUENCE_0935 TCR045 TRAV CDR3 3 Tcell_045 Mus_musculus
CAQGRALIF

SEQ ID NO: 878 - protein, 14 aa, Mus musculus
  >SEQUENCE_0936 TCR046 TRAV CDR3 3 Tcell_046 Mus_musculus
CALSPNSAGNKLTF

SEQ ID NO: 879 - protein, 12 aa, Mus musculus
  >SEQUENCE_0937 TCR047 TRAV CDR3 3 Tcell_047 Mus_musculus
CVLKGSNNRIFF

SEQ ID NO: 880 - protein, 13 aa, Mus musculus
  >SEQUENCE_0938 TCR048 TRAV CDR3 3 Tcell_048 Mus_musculus
CAARTQVVGQLTF

SEQ ID NO: 881 - protein, 13 aa, Mus musculus
  >SEQUENCE_0939 TCR049 TRAV CDR3 3 Tcell_049 Mus_musculus
CALGGTGYQNFYF

SEQ ID NO: 882 - protein, 16 aa, Mus musculus
  >SEQUENCE_0940 TCR050 TRAV CDR3 3 Tcell_050 Mus_musculus
CAVSDPLSGSFNKLTF

SEQ ID NO: 883 - protein, 16 aa, Mus musculus
  >SEQUENCE_0941 TCR051 TRAV CDR3 3 Tcell_051 Mus_musculus
CAVSDPLSGSFNKLTF

SEQ ID NO: 884 - protein, 14 aa, Mus musculus
  >SEQUENCE_0942 TCR052 TRAV CDR3 3 Tcell_052 Mus_musculus
CAVGYGSSGNKLIF

SEQ ID NO: 885 - protein, 14 aa, Mus musculus
  >SEQUENCE_0943 TCR053 TRAV CDR3 3 Tcell_053 Mus_musculus
CAIEWGNYAQGLTF

SEQ ID NO: 886 - protein, 13 aa, Mus musculus
  >SEQUENCE_0944 TCR054 TRAV CDR3 3 Tcell_054 Mus_musculus
CAATEGNTNKVVF

SEQ ID NO: 887 - protein, 14 aa, Mus musculus
  >SEQUENCE_0945 TCR055 TRAV CDR3 3 Tcell_055 Mus_musculus
CALSTTASLGKLQF

SEQ ID NO: 888 - protein, 13 aa, Mus musculus
  >SEQUENCE_0946 TCR056 TRAV CDR3 2 Tcell_056 Mus_musculus
CAASLQGGRALIF

SEQ ID NO: 889 - protein, 15 aa, Mus musculus
  >SEQUENCE_0947 TCR057 TRAV CDR3 2 Tcell_057 Mus_musculus
CAASKGGSALGRLHF

SEQ ID NO: 890 - protein, 14 aa, Mus musculus
  >SEQUENCE_0948 TCR058 TRAV CDR3 2 Tcell_058 Mus_musculus
CAVSAGYNQGKLIF

SEQ ID NO: 891 - protein, 13 aa, Mus musculus
  >SEQUENCE_0949 TCR059 TRAV CDR3 2 Tcell_059 Mus_musculus
CAASASMGYKLTF

SEQ ID NO: 892 - protein, 11 aa, Mus musculus
  >SEQUENCE_0950 TCR060 TRAV CDR3 2 Tcell_060 Mus_musculus
CAAEFNNRIFF

SEQ ID NO: 893 - protein, 14 aa, Mus musculus
  >SEQUENCE_0951 TCR061 TRAV CDR3 2 Tcell_061 Mus_musculus
CAVSMSQGGRALIF

SEQ ID NO: 894 - protein, 16 aa, Mus musculus
  >SEQUENCE_0952 TCR062 TRAV CDR3 2 Tcell_062 Mus_musculus
CIVTAAPYQGGRALIF

SEQ ID NO: 895 - protein, 13 aa, Mus musculus
  >SEQUENCE_0953 TCR063 TRAV CDR3 2 Tcell_063 Mus_musculus
CALSEPVVGQLTF

SEQ ID NO: 896 - protein, 14 aa, Mus musculus
  >SEQUENCE_0954 TCR064 TRAV CDR3 2 Tcell_064 Mus_musculus
CAVSERNYAQGLTF

SEQ ID NO: 897 - protein, 14 aa, Mus musculus
  >SEQUENCE_0955 TCR065 TRAV CDR3 2 Tcell_065 Mus_musculus
CAASKPSNTNKVVF

SEQ ID NO: 898 - protein, 15 aa, Mus musculus
  >SEQUENCE_0956 TCR066 TRAV CDR3 2 Tcell_066 Mus_musculus
CALGPSDYSNNRLTL

SEQ ID NO: 899 - protein, 15 aa, Mus musculus
  >SEQUENCE_0957 TCR067 TRAV CDR3 2 Tcell_067 Mus_musculus
CAMSPSSSSFSKLVF

SEQ ID NO: 900 - protein, 13 aa, Mus musculus
  >SEQUENCE_0958 TCR068 TRAV CDR3 2 Tcell_068 Mus_musculus
CALGDLSNNRIFF

SEQ ID NO: 901 - protein, 14 aa, Mus musculus
  >SEQUENCE_0959 TCR069 TRAV CDR3 2 Tcell_069 Mus_musculus
CAAEASSGSWQLIF

SEQ ID NO: 902 - protein, 14 aa, Mus musculus
  >SEQUENCE_0960 TCR070 TRAV CDR3 2 Tcell_070 Mus_musculus
CATGLSGSFNKLTF

SEQ ID NO: 903 - protein, 14 aa, Mus musculus
  >SEQUENCE_0961 TCR071 TRAV CDR3 2 Tcell_071 Mus_musculus
CAASVTGNTGKLIF

SEQ ID NO: 904 - protein, 101 aa, Mus musculus
  >SEQUENCE_0962 TCR001 TRAV Full length 17 Tcell_001 Mus_musculus
QQKVQQSPESLSVPEGGMASFNCTSSDRNFQYFWWYRQHSGEGPKALMSIFSDGDKKEGRFTAHLNKASLHVSLHIRDSQPSDSALYFCCAAPSNYNVLYF

SEQ ID NO: 905 - protein, 104 aa, Mus musculus
  >SEQUENCE_0963 TCR002 TRAV Full length 6 Tcell_002 Mus_musculus
AQSVTQPDARVTVSEGASLQLRCKYSYSATPYLFWYVQYPRQGLQLLLKYYSGDPVVQGVNGFEAEFSKSNSSFHLRKASVHWSDSAVYFCCAVRNTGYQNFYF

SEQ ID NO: 906 - protein, 104 aa, Mus musculus
  >SEQUENCE_0964 TCR003 TRAV Full length 7 Tcell_003 Mus_musculus
GDSVTQTEGQVALSEEDFLTIHCNYSASGYPALFWYVQYPGEGPQFLFRASRDKEKGSSRGFEATYDKGTTSFHLRKASVQESDSAVYYCCALGDHNYAQGLTF

SEQ ID NO: 907 - protein, 101 aa, Mus musculus
  >SEQUENCE_0965 TCR004 TRAV Full length 4 Tcell_004 Mus_musculus
DAKTTQPDSMESTEGETVHLPCSHATISGNEYIYWYRQVPLQGPEYVTHGLQQNTTNSMAFLAIASDRKSSTLILTHVSLRDAAVYHCCILRGSALGRLHF

SEQ ID NO: 908 - protein, 105 aa, Mus musculus
  >SEQUENCE_0966 TCR005 TRAV Full length 6 Tcell_005 Mus_musculus
AQSVTQPDARVTVSEGASLQLRCKYSYSATPYLFWYVQYPRQGLQLLLKYYSGDPVVQGVNGFEAEFSKSNSSFHLRKASVHWSDSAVYFCCAVSASTGNYKYVF

SEQ ID NO: 909 - protein, 104 aa, Mus musculus
  >SEQUENCE_0967 TCR006 TRAV Full length 5 Tcell_006 Mus_musculus
GEKVEQHQSTLSVREGDSAVINCTYTDTASSYFPWYKQEAGKSLHFVIDIRSNVDRKQSQRLTVLLDKKAKRFSLHITATQPEDSAIYFCCAASRSSSFSKLVF

SEQ ID NO: 910 - protein, 104 aa, Mus musculus
  >SEQUENCE_0968 TCR007 TRAV Full length 2 Tcell_007 Mus_musculus
QQKVQQSPESLTVSEGAMASLNCTFSDGTSDNFRWYRQHSGKGLEMLVSIFSDGEKEEGRFTAHLNRASLHVSLHIREPQPSDSAVYLCCAVSMRAGNTGKLIF

SEQ ID NO: 911 - protein, 105 aa, Mus musculus
  >SEQUENCE_0969 TCR008 TRAV Full length 3 Tcell_008 Mus_musculus
QQQVRQSPQSLTVWEGETAILNCSYEDSTFNYFPWYQQFPGEGPALLISIRSVSDKKEDGRFTIFFNKREKKLSLHITDSQPGDSATYFCCAASGYGSSGNKLIF

SEQ ID NO: 912 - protein, 108 aa, Mus musculus
  >SEQUENCE_0970 TCR009 TRAV Full length 2 Tcell_009 Mus_musculus
GDSVTQTEGLVTLTEGLPVMLNCTYQTAYSTFLFWYVQHLNEAPKLLLKSSTDNKRTEHQGFHATLHKSSSSFHLQKSSAQLSDSALYYCCALSDREPQQGTGSKLSF

SEQ ID NO: 913 - protein, 105 aa, Mus musculus
  >SEQUENCE_0971 TCR010 TRAV Full length 2 Tcell_010 Mus_musculus
GDSVTQTEGQVALSEEDFLTIHCNYSASGYPALFWYVQYPGEGPQFLFRASRDKEKGSSRGFEATYDKGTTSFHLRKASVQESDSAVYYCCALGDKSTNTGKLTF

SEQ ID NO: 914 - protein, 102 aa, Mus musculus
  >SEQUENCE_0972 TCR011 TRAV Full length 2 Tcell_011 Mus_musculus
GQQVKQSSPSLTVQEGGILILNCDYENDMFDYFAWYKKYPDNSPTLLISVRSNVDKREDGRFTVFLNKSGKHFSLHITASQPEDTAVYLCCAATGGNNKLTF

SEQ ID NO: 915 - protein, 103 aa, Mus musculus
  >SEQUENCE_0973 TCR012 TRAV Full length 4 Tcell_012 Mus_musculus
DAKTTQPDSMESTEGETVHLPCSHATISGNEYIYWYRQVPLQGPEYVTHGLQQNTTNSMAFLAIASDRKSSTLILTHVSLRDAAVYHCCILRANQGGSAKLIF

SEQ ID NO: 916 - protein, 101 aa, Mus musculus
  >SEQUENCE_0974 TCR013 TRAV Full length 2 Tcell_013 Mus_musculus
QQKVQQSPESLTVSEGAMASLNCTFSDGTSDNFRWYRQHSGKGLEVLVSIFSDGEKEEGRFTAHLNRASLHVSLHIREPQPSDSAVYLCCAVGGTGSKLSF

SEQ ID NO: 917 - protein, 103 aa, Mus musculus
  >SEQUENCE_0975 TCR014 TRAV Full length 2 Tcell_014 Mus_musculus
QQQVRQSPQSLTVWEGETAILNCSYEDSTFNYFPWYQQFPGEGPALLISIRSVSDKKEDGRFTIFFNKREKKLSLHITDSQPGDSATYFCCAARGTGNYKYVF

SEQ ID NO: 918 - protein, 99 aa, Mus musculus
  >SEQUENCE_0976 TCR015 TRAV Full length 2 Tcell_015 Mus_musculus
GQQVQQSPASLVLQEGENAELQCNFSTSLNSMQWFYQRPEGSLVSLFYNPSGTKQSGRLTSTTVIKERRSSLHISSSQITDSGTYLCCAWLTGNYKYVF

SEQ ID NO: 919 - protein, 103 aa, Mus musculus
  >SEQUENCE_0977 TCR016 TRAV Full length 2 Tcell_016 Mus_musculus
SQWGEENLQALSIQEGEDVTMNCSYKTYTTVVQWYRQKSGKGPALIILIRSNEREKRSGRLRATLDTSSQSSSLSITGTLATDTAVYFCCATDRGSALGRLHF

SEQ ID NO: 920 - protein, 103 aa, Mus musculus
  >SEQUENCE_0978 TCR017 TRAV Full length 5 Tcell_017 Mus_musculus
GEQVEQRPPHLSVREGDSAVITCTYTDPNSYYFFWYKQEPGASLQLLMKVFSSTEINEGQGFTVLLNKKDKRLSLNLTAAHPGDSAAYFCCAVKDTNTGKLTF

SEQ ID NO: 921 - protein, 104 aa, Mus musculus
  >SEQUENCE_0979 TCR018 TRAV Full length 2 Tcell_018 Mus_musculus
GEQVEQLPSILRVQERSSASINCTYENSASNYFPWYKQEPGENPKLIIDIRSNMERKQIQGLIVLLDKKAKRFSLHITDTQPADSAMYFCCAASARGTGSKLSF

SEQ ID NO: 922 - protein, 102 aa, Mus musculus
  >SEQUENCE_0980 TCR019 TRAV Full length 2 Tcell_019 Mus_musculus
QQKVQQSPESLIVPEGGMASLNCTSSDRNVDYFWWYRQHSGKSPKMLMSIFSNGEKEEGRFTVHLNKASLHTSLHIRDSQPSDSALYLCCAALSGSFNKLTF

SEQ ID NO: 923 - protein, 88 aa, Mus musculus
  >SEQUENCE_0981 TCR020 TRAV Full length 10 Tcell_020 Mus_musculus
GAFIICTYTDSATAYFYWYKQEPGAGLQLLMSVFSNVDRKEEQGLTVLLNKKYKRLSLNLTAAHPGDSAVYFCCAVSQATGGNNKLTF

SEQ ID NO: 924 - protein, 100 aa, Mus musculus
  >SEQUENCE_0982 TCR021 TRAV Full length 3 Tcell_021 Mus_musculus
GQGVEQPDNLMSVEGTFARVNCTYSTSGFNGLSWYQQREGHAPVFLSYVVLDGLKDSGHFSTFLSRSNGYSYLLLTELQIKDSASYLCCAVMDSNYQLIW

SEQ ID NO: 925 - protein, 107 aa, Mus musculus
  >SEQUENCE_0983 TCR022 TRAV Full length 3 Tcell_022 Mus_musculus
GDSVTQTEGQVTVSESKSLIINCTYSTTSIAYPNLFWYVRYPGEGLQLLLKVITAGQKGSSRGFEATYNKETTSFHLQKASVQESDSAVYYCCALGAGSSNTNKVVF

SEQ ID NO: 926 - protein, 100 aa, Mus musculus
  >SEQUENCE_0984 TCR023 TRAV Full length 3 Tcell_023 Mus_musculus
AQKVTQTQTSISVMEKTTVTMDCVYETQDSSYFLFWYKQTASGEIVFLIRQDSYKKENATVGHYSLNFQKPKSSIGLIITATQIEDSAVYFCCAMREGAT

SEQ ID NO: 927 - protein, 104 aa, Mus musculus
  >SEQUENCE_0985 TCR024 TRAV Full length 3 Tcell_024 Mus_musculus
GDSVTQTEGQVALSEEDFLTIHCNYSASGYPALFWYVQYPGEGPQFLFRASRDKEKGSSRGFEATYNKETTSFHLQKASVQESDSAVYYCCALRANTGYQNFYF

SEQ ID NO: 928 - protein, 103 aa, Mus musculus
  >SEQUENCE_0986 TCR025 TRAV Full length 2 Tcell_025 Mus_musculus
GDSVTQTEGLVTLTEGLPVMLNCTYQTIYSNPFLFWYVHYLNESPRLLLKSSTDNKRTEHQGFHATLHKSSSSFHLQKSSAQLSDSALYYCCALRNYAQGLTF

SEQ ID NO: 929 - protein, 102 aa, Mus musculus
  >SEQUENCE_0987 TCR026 TRAV Full length 2 Tcell_026 Mus_musculus
GDQVEQSPSALSLHEGTDSALRCNFTTTMRSVQWFRQNSRGSLISLFYLASGTKENGRLKSAFDSKERRYSTLHIRDAQLEDSGTYFCCAAEASSGSWQLIF

SEQ ID NO: 930 - protein, 103 aa, Mus musculus
  >SEQUENCE_0988 TCR027 TRAV Full length 2 Tcell_027 Mus_musculus
GDSVTQKQGQVTLSEDDFLFINCTYSTTTYPTLLWYVQYPGQGPQLLLKVTTANNKGISRGFEATYDKGTTSFHLQKASVQESDSAVYFCCALVRYNQGKLIF

SEQ ID NO: 931 - protein, 103 aa, Mus musculus
  >SEQUENCE_0989 TCR028 TRAV Full length 2 Tcell_028 Mus_musculus
GDSVTQKQGQVTLSEDDFLFINCTYSTTTYPTLLWYVQYPGQGPQLLLKVTTANNKGISRGFEATYDKGTTSFHLQKASVQESDSAVYFCCALVDWSNNRIFF

SEQ ID NO: 932 - protein, 102 aa, Mus musculus
  >SEQUENCE_0990 TCR029 TRAV Full length 2 Tcell_029 Mus_musculus
GDQVEQSPSALSLHEGTGSALRCNFTTTMRAVQWFRKNSRGSLINLFYLASGTKENGRLKSAFDSKERYSTLHIRDAQLEDSGTYFCCAAVDRGSALGRLHF

SEQ ID NO: 933 - protein, 104 aa, Mus musculus; identical to SEQ ID NO: 905
  >SEQUENCE_0991 TCR030 TRAV Full length 2 Tcell_030 Mus_musculus

SEQ ID NO: 934 - protein, 102 aa, Mus musculus
  >SEQUENCE_0992 TCR031 TRAV Full length 2 Tcell_031 Mus_musculus
QEKVQQSPESLIVPEGAMSSLNCTFSNSASQSIWWYQQHPGKGPEALISIFSNGNKKEGRLTVYLNRASLHVSLHIRDSQPSDSAVYLCCAVSGNSNNRIFF

SEQ ID NO: 935 - protein, 105 aa, Mus musculus
  >SEQUENCE_0993 TCR032 TRAV Full length 2 Tcell_032 Mus_musculus
AQSVTQPDARVTVSEGASLQLRCKYSYSATPYLFWYVQYPRQGLQLLLKYYSGDPVVQGVNGFEAEFSKSNSSFHLRKASVHWSDSAVYFCCAVSARNAGAKLTF

SEQ ID NO: 936 - protein, 103 aa, Mus musculus
  >SEQUENCE_0994 TCR033 TRAV Full length 2 Tcell_033 Mus_musculus
GDSVIQMQGQVTLSENDFLFINCTYSTTGYPTLFWYVQYSGEGPQLLLQVTTANNKGSSRGFEATYDKGTTSFHLQKTSVQEIDSAVYYCCAMSDLAGNKLTF

SEQ ID NO: 937 - protein, 104 aa, Mus musculus
  >SEQUENCE_0995 TCR034 TRAV Full length 2 Tcell_034 Mus_musculus
GDSVTQTEGQVALSEEDFLTIHCNYSASGYPALFWYVQYPGEGPQFLFRASRDKEKGSSRGFEATYDKGTTSFHLRKASVQESDSAVYYCCALANMDSNYQLIW

SEQ ID NO: 938 - protein, 103 aa, Mus musculus
  >SEQUENCE_0996 TCR035 TRAV Full length 2 Tcell_035 Mus_musculus
DSVTQTEGLVTLTEGLPVMLNSTYQTIYSNPYLFWYVQHLNESPRLLLKNFTDNKRTEHQGFHATLHKSSSSSHLQKSSAQLSDSALYCCCALASGGNYKPTF

SEQ ID NO: 939 - protein, 107 aa, Mus musculus
  >SEQUENCE_0997 TCR036 TRAV Full length 2 Tcell_036 Mus_musculus
AQSVTQPDARVTVSEGASLQLRCKYSYFGTPYLFWYVQYPRQGLQLLLKYYPGDPVVQGVNGFEAEFSKSNSSFHLRKASVHWSDWAVYFCCAVSPMTNSAGNKLTF

SEQ ID NO: 940 - protein, 102 aa, Mus musculus
  >SEQUENCE_0998 TCR037 TRAV Full length 2 Tcell_037 Mus_musculus
GEQVEQLPSSLIVQEGASVLINCSYTDSASVYFPWYKQEPGKRLQFIIDIRSNMERKQNQRLTLLFDKKTKHLSLHITATQPGDSAIYFCCSATTGGYKVVF

SEQ ID NO: 941 - protein, 106 aa, Mus musculus
  >SEQUENCE_0999 TCR038 TRAV Full length 4 Tcell_038 Mus_musculus
AQKVTQTQTSISVVEKTTVTMDCVYETRDSSYFLFWYKQTASGEIVFLIRQDSYKKENATVGHYSLNFQKPKSSIGLIITATQIEDSAVYFCCAMREGNNNNAPRF

SEQ ID NO: 942 - protein, 103 aa, Mus musculus
  >SEQUENCE_1000 TCR039 TRAV Full length 12 Tcell_039 Mus_musculus
GQQVQQSPSSLVLQEGENAELQCNFSSTATQLQWFYQSPGGSLVSLLSNPSGTKHTGRLTSTTVTKERRSSLHISSSQTTDSGTYLCCATLIDRGSALGRLHF

SEQ ID NO: 943 - protein, 103 aa, Mus musculus
  >SEQUENCE_1001 TCR040 TRAV Full length 5 Tcell_040 Mus_musculus
GDSVTQTEGLVTLTEGLPVMLNCTYQTAYSTFLFWYVQHLNEAPKLLLKSSTDNKRTEHQGFHATLHKSSSSFHLQKSSAQLSDSALYYCCAATTASLGKLQF

SEQ ID NO: 944 - protein, 109 aa, Mus musculus
  >SEQUENCE_1002 TCR041 TRAV Full length 4 Tcell_041 Mus_musculus
AQKVTQTQTSISVMEKTTVTMDCVYETQDSSYFLFWYKQTASGEIVFLIRQDSYKKENATVGHYSLNFQKPKSSIGLIITATQIEDSAVYFCCAMREALSGGSNAKLTF

SEQ ID NO: 945 - protein, 102 aa, Mus musculus
  >SEQUENCE_1003 TCR042 TRAV Full length 4 Tcell_042 Mus_musculus
GDSVTQTEGLVTLTEGLPVMLNCTYQTAYSTFLFWYVQHLNEAPKLLLKSSTDNKRTEHQGFHATLHKSSSSFHLQKSSAQLSDSALYYCCAQTSSGQKLVF

SEQ ID NO: 946 - protein, 103 aa, Mus musculus; identical to SEQ ID NO: 931
  >SEQUENCE_1004 TCR043 TRAV Full length 3 Tcell_043 Mus_musculus

SEQ ID NO: 947 - protein, 104 aa, Mus musculus
  >SEQUENCE_1005 TCR044 TRAV Full length 5 Tcell_044 Mus_musculus
GDSVTQTEGLVTLTEGLPVMLNCTYQSTYSPFLFWYVQHLNEAPKLLLKSFTDNKRPEHQGFHATLHKSSSSFHLQKSSAQLSDSALYYCCALRASSGSWQLIF

SEQ ID NO: 948 - protein, 99 aa, Mus musculus
  >SEQUENCE_1006 TCR045 TRAV Full length 3 Tcell_045 Mus_musculus
GDSVTQTEGQVALSEEDFLTIHCNYSASGYPALFWYVQYPGEGPQFLFRASRDKEKGSSRGFEATYDKGTTSFHLRKASVQESDSAVYYCCAQGRALIF

SEQ ID NO: 949 - protein, 105 aa, Mus musculus
  >SEQUENCE_1007 TCR046 TRAV Full length 3 Tcell_046 Mus_musculus
GDSVTQTEGLVTVTEGLPVMLNCTYQTAYSDVAFFWYVQYLNEAPKLLLRSSTDNKRTEHQGFHATLHKSSSSFHLQKSSVQLSDSALYYCCALSPNSAGNKLTF

SEQ ID NO: 950 - protein, 103 aa, Mus musculus
  >SEQUENCE_1008 TCR047 TRAV Full length 3 Tcell_047 Mus_musculus
AQSVTQPDARVTVSEGASLQLRCKYSYSGTPYLFWYVQYPRQGLQLLLKYYSGDPVVQGVNGFEAEFSKSDSSFHLRKASVHWSDSAVYFCCVLKGSNNRIFF

SEQ ID NO: 951 - protein, 103 aa, Mus musculus
  >SEQUENCE_1009 TCR048 TRAV Full length 3 Tcell_048 Mus_musculus
QQQVRQSSQSLTVWEGETAILNCSYENSAFDYFPWYQQFPGEGPALLIAIRSVSDKKEDGRFTIFFNKREKNLSLHIKDSQPGDSATYFCCAARTQVVGQLTF

SEQ ID NO: 952 - protein, 105 aa, Mus musculus
  >SEQUENCE_1010 TCR049 TRAV Full length 3 Tcell_049 Mus_musculus
GDSVTQTEGQVTVSESKSLIINCTYSTTSIAYPNLFWYVRYPGEGLQLLLKVITAGQKGSSRGFEATYNKETTSFHLQKASVQESDSAVYYCCALGGTGYQNFYF

SEQ ID NO: 953 - protein, 105 aa, Mus musculus
  >SEQUENCE_1011 TCR050 TRAV Full length 3 Tcell_050 Mus_musculus
QQKVQQSPESLSVPEGGMASLNCTSSDRNFQYFWWYRQHSGEGPKALMSIFSDGDKKEGRFTAHLNKASLHVSLHIRDSQPSDSALYFCCAVSDPLSGSFNKLTF

SEQ ID NO: 954 - protein, 105 aa, Mus musculus; identical to SEQ ID NO: 953
  >SEQUENCE_1012 TCR051 TRAV Full length 3 Tcell_051 Mus_musculus

SEQ ID NO: 955 - protein, 104 aa, Mus musculus
  >SEQUENCE_1013 TCR052 TRAV Full length 3 Tcell_052 Mus_musculus
GEQVEQRPPHLSVREGDSAFITCTYTDPNSYYFFWYKQEPGASLQLLMKVFSSTEINEGQGFTVLLNKKDKRLSLNLTAAHPGDSAAYFCCAVGYGSSGNKLIF

SEQ ID NO: 956 - protein, 101 aa, Mus musculus
  >SEQUENCE_1014 TCR053 TRAV Full length 3 Tcell_053 Mus_musculus
GQQVQQSPASLVLQEGENAELQCNFSSTATRLQWFYQHPGGRLVSLFYNPSGTKHTGRLTSTTVTNERRGSLHISSSQTTDSGTYFCCAIEWGNYAQGLTF

SEQ ID NO: 957 - protein, 102 aa, Mus musculus
  >SEQUENCE_1015 TCR054 TRAV Full length 3 Tcell_054 Mus_musculus
QQKVQQSPESLIVPEGGMASLNCTSSDRNVDYFWWYRQHSGKSPKMLMSIFSNGEKEEGRFTVHLNKASLHTSLHIRDSQPSDSALYLCCAATEGNTNKVVF

SEQ ID NO: 958 - protein, 105 aa, Mus musculus
  >SEQUENCE_1016 TCR055 TRAV Full length 3 Tcell_055 Mus_musculus
AQSVTQPDARVTVSEGASLQLRCKYSYSATPYLFWYVQYPRQGLQLLLKYYSGDPVVQGVNSFEAEFSKSNSSFHLQKASVHWSDSAVYFCCALSTTASLGKLQF

SEQ ID NO: 959 - protein, 103 aa, Mus musculus
  >SEQUENCE_1017 TCR056 TRAV Full length 2 Tcell_056 Mus_musculus
QQQVRQSPQSLTVWEGETAILNCSYEDSTFNYFPWYQQFPGEGPALLISIRSVSDKKEDGRFTIFFNKREKKLSLHITDSQPGDSATYFCCAASLQGGRALIF

SEQ ID NO: 960 - protein, 104 aa, Mus musculus
  >SEQUENCE_1018 TCR057 TRAV Full length 2 Tcell_057 Mus_musculus
QQKVQQSPESLIVPEGGMASLNCTSSDRNVDYFWWYRQHSGKSPKMLMSIFSNGEKEEGRFTVHLNKASLHTSLHIRDSQPSDSALYLCCAASKGGSALGRLHF

SEQ ID NO: 961 - protein, 104 aa, Mus musculus
  >SEQUENCE_1019 TCR058 TRAV Full length 2 Tcell_058 Mus_musculus
GEQVEQRPPHLSVREGDSAVITCTYTDPNSYYFFWYKQEPGASLQLLMKVFSSTEINEGQGFTVLLNKKDKRLSLNLTAAHPGDSAAYFCCAVSAGYNQGKLIF

SEQ ID NO: 962 - protein, 103 aa, Mus musculus
  >SEQUENCE_1020 TCR059 TRAV Full length 2 Tcell_059 Mus_musculus
QQQVRQSSQSLTVWEGETAILNCSYENSAFDYFPWYQQFPGEGPALLIAIRSVSDKKEDGRFTIFFNKREKNLSLHIKDSQPGDSATYFCCAASASMGYKLTF

SEQ ID NO: 963 - protein, 98 aa, Mus musculus
  >SEQUENCE_1021 TCR060 TRAV Full length 2 Tcell_060 Mus_musculus
GDQVEQSPSALSLHEGTGSALRCNFTTTMRAVQWFRKNSRGSLINLFYLASGTKENGRLKSAFDSKERYSTLHIRDAQLEDSGTYFCCAAEFNNRIFF

SEQ ID NO: 964 - protein, 105 aa, Mus musculus
  >SEQUENCE_1022 TCR061 TRAV Full length 2 Tcell_061 Mus_musculus
AQSVTQPDARVTVSEGASLQLRCKYSYFGTPYLFWYVQYPRQGLQLLLKYYPGDPVVQGVNGFEAEFSKSNSSFHLRKASVHWSDWAVYFCCAVSMSQGGRALIF

SEQ ID NO: 965 - protein, 104 aa, Mus musculus
  >SEQUENCE_1023 TCR062 TRAV Full length 2 Tcell_062 Mus_musculus
LAKTTQPPSMEAYEGQEVNVSCSHTNIATSEYIYWYRQVPHQGPQFIIQGYKDYVVNEVASLFISADRKLSTLSLPWVSLRDAAVYYCCIVTAAPYQGGRALIF

SEQ ID NO: 966 - protein, 105 aa, Mus musculus
  >SEQUENCE_1024 TCR063 TRAV Full length 2 Tcell_063 Mus_musculus
GDSVTQTEGQVTVSESKSLIINCTYSATSIGYPNLFWYVRYPGEGLQLLLKVITAGQKGSSRGFEATYNKEATSFHLQKASVQESDSAVYYCCALSEPVVGQLTF

SEQ ID NO: 967 - protein, 104 aa, Mus musculus
  >SEQUENCE_1025 TCR064 TRAV Full length 2 Tcell_064 Mus_musculus
GEQVEQRPPHLSVREGDSAFITCTYTDPNSYYFFWYKQEPGASLQLLMKVFSSTEINEGQGFTVLLNKKDKRLSLNLTAAHPGDSAAYFCCAVSERNYAQGLTF

SEQ ID NO: 968 - protein, 104 aa, Mus musculus
  >SEQUENCE_1026 TCR065 TRAV Full length 2 Tcell_065 Mus_musculus
GEKVEQHESTLSVREGDSAVINCTYTDTASSYFPWYKQEAGKGLHFVIDIRSNVDRKQSQRLIVLLDKKAKRFSLHITATQPEDSAIYFCCAASKPSNTNKVVF

SEQ ID NO: 969 - protein, 105 aa, Mus musculus
  >SEQUENCE_1027 TCR066 TRAV Full length 2 Tcell_066 Mus_musculus
GDSVTQTEGQVALSEEDFLTIHCNYSASGYPALFWYVQYPGEGPQFLFRASRDKEKGSSRGFEATYDKGTTSFHLRKASVQESDSAVYYCCALGPSDYSNNRLTL

SEQ ID NO: 970 - protein, 107 aa, Mus musculus
  >SEQUENCE_1028 TCR067 TRAV Full length 2 Tcell_067 Mus_musculus
AQKVTQTQTSISVMEKTTVTMDCVYETQDSSYFLFWYKQTASGEIVFLIRQDSYKKENATVGHYSLNFQKPKSSIGLIITATQIEDSAVYFCCAMSPSSSSFSKLVF

SEQ ID NO: 971 - protein, 105 aa, Mus musculus
  >SEQUENCE_1029 TCR068 TRAV Full length 2 Tcell_068 Mus_musculus
GDSVTQTEGQVTVSESKSLIINCTYSTTSIAYPNLFWYVRYPGEGLQLLLKVITAGQKGSSRGFEATYNKETTSFHLQKASVQESDSAVYYCCALGDLSNNRIFF

SEQ ID NO: 972 - protein, 101 aa, Mus musculus
  >SEQUENCE_1030 TCR069 TRAV Full length 2 Tcell_069 Mus_musculus
GDKVKQSPSALSLQEGTNSALRCNFSIAATTVQWFLQNPRGSLINLFYLVPGTKENGRLKSTFNSKESYSTLHIRDAQLEDSGTYFCCAAEASSGSWQLIF

SEQ ID NO: 973 - protein, 103 aa, Mus musculus
  >SEQUENCE_1031 TCR070 TRAV Full length 2 Tcell_070 Mus_musculus
SQWGEENLQALSIQEGEDVTMNCSYKTYTTVVQWYRQKSGKGPAQLILIRSNEREKRSGRLRATLDTSSQSSSLSITGTLATDTAVYFCCATGLSGSFNKLTF

SEQ ID NO: 974 - protein, 104 aa, Mus musculus
  >SEQUENCE_1032 TCR071 TRAV Full length 2 Tcell_071 Mus_musculus
GEQVEQLPSILRVQERSSASINCTYENSASNYFPWYKQEPGENPKLIIDIRSNMERKQIQGLIVLLDKKAKRFSLHITDTQPADSAMYFCCAASVTGNTGKLIF